(19) United States
(12) Patent Application Publication
(10) Pub. No.: US 2024/0425846 A1
(43) Pub. Date: Dec. 26, 2024

(54) METHODS OF BIOMOLECULE DISPLAY

(71) Applicant: United Kingdom Research and Innovation, Swindon, Wiltshire (GB)

(72) Inventors: Benjamin POREBSKI, Swindon (GB); Philipp HOLLIGER, Swindon (GB)

(21) Appl. No.: 18/690,345

(22) PCT Filed: Sep. 6, 2022
(86) PCT No.: PCT/EP2022/074731

§ 371 Date: Mar. 8, 2024

(30) Foreign Application Priority Data
Sep. 10, 2021 (GB) 2112907.7

(51) Int. Cl. C12N 15/10; C12Q 1/34; C12Q 1/6834; C12Q 1/6844; C12Q 1/6874

(52) U.S. Cl. CPC C12N 15/1044; C12Q 1/34; C12Q 1/6834; C12Q 1/6844; C12Q 1/6874; G01N 2333/924

(57) ABSTRACT

The invention relates to methods of displaying biomolecules on substrates, for instance on the surface of flow cells. The invention relates to upstream, downstream, or direct methods for displaying XNA molecules, RNA molecules, and/or polypeptides on the substrate. The invention further relates to substrates displaying biomolecules that are obtained or obtainable by the methods of the invention.

### FIELD OF THE INVENTION

The present invention relates to methods of displaying biomolecules on substrates, for instance on the surface of flow cells. The invention relates to upstream, downstream, or direct methods for displaying xeno nucleic acid (XNA) molecules, RNA molecules, and/or polypeptides on a substrate. The invention further relates to substrates displaying biomolecules that are obtained or obtainable by the methods of the invention.

### BACKGROUND OF THE INVENTION

Platforms which enable high-through analysis of biological molecules, for instance analysis of binding affinities or other properties, are important for drug discovery. Flow cells are commonly used as substrates for displaying DNA molecules which can then be interrogated to obtain both positional and sequence information.

Attempts have been made to make use of flow cells displaying RNA molecules or polypeptides. For instance, some methods make use of DNA clusters immobilised to a flow cell to produce RNA that is non-covalently tethered to the DNA clusters via a stalled RNA polymerase. The RNA may then be translated. Such methods include those disclosed in WO2014/189768, Layton et al. (Layton et al., 2019, Molecular Cell 73, 1075-1082), and US2019/0112730. However, there are known drawbacks to these approaches. For instance, these complexes are not covalently linked to the flow cell and can decompose over time and need to be assayed using loss-of-signal normalization techniques. In addition, any analysis that requires conditions that could denature the complexes cannot be carried out. For instance, high temperature, chemical denaturants, and low or high concentrations of magnesium will disassociate the complexes. Low concentrations of magnesium can cause the disassociation of ribosomes from complexes. High concentrations of magnesium can cause the disassociation of RNA polymerases from complexes. Thus, such display techniques have limitations.

Svensen et al. describe a method for converting flow cell-bound clusters of identical DNA strands generated by the Illumina DNA sequencing technology into clusters of complementary RNA, and subsequently peptide clusters (Chembiochem. 2016 Sep. 2: 17 (17): 1628-1635. doi: 10.1002/cbic.201600298). The method requires the modification of the flow cell-bound primers with ribonucleotides to enable them to be used by poliovirus 3Dpol polymerase. The yield of the RNA produced is not optimal and hence the yield of polypeptides produced could be increased.

As such, there is a need in the art for methods of displaying biomolecules that overcome the aforementioned issues.

Moriizumi et al. provide findings that relate to in vitro translation (Moriizumi, Yoshiki, et al. “Osmolyte-enhanced protein synthesis activity of a reconstituted translation system.” ACS synthetic biology 8.3 (2019): 557-567).

### SUMMARY OF THE INVENTION

The inventors provide herein methods which enable the production of high-throughput drug discovery platforms. The inventors create substrate-bound libraries of biological molecules, including XNA, RNA, and polypeptides, and show that these libraries may be interrogated. For instance, by the measurement of binding affinities or enzymatic activity.

Specifically, the inventors have been able to sequence and screen a library of up to 10⁸ variants with replicate measurements in 2-3 days. Paired sequence-function information can be generated for all library variants.

As such, the platform disclosed herein generates an unprecedented amount of high-resolution data which may, for instance, be used in conjunction with machine learning when engineering therapeutic drugs.

In an aspect of the invention, there is provide a method of displaying a non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for polymerisation, wherein
   the primer for polymerisation is a DNA primer immobilised on the substrate such a bridge is formed during polymerisation,
   the product of the polymerisation is a chain of non-DNA nucleotides that is immobilised on the substrate via the primer, and
   the nucleic acid polymerase is a polymerase capable of acting upon a DNA primer to synthesise a non-DNA nucleic acid molecule that is complementary to a single-stranded nucleic acid template; and
iii) removing the first nucleic acid to result in display of the second nucleic acid on the substrate.

The second nucleic acid may be an RNA molecule. The nucleic acid polymerase may comprise an amino acid sequence having at least 36% identity to the amino acid sequence of SEQ ID NO: 1, wherein said amino acid sequence comprises a Y409 and an E664 mutation relative to the amino acid sequence of SEQ ID NO:1. The Y409 mutation may be Y409N or Y409G and the E664 mutation may be E664K or E664Q. In a particular embodiment, the Y409 mutation is Y409G and the E664 mutation is E664K. The amino acid sequence of the nucleic acid polymerase may comprise SEQ ID NO: 3.

The second nucleic acid may be an XNA molecule. For instance, the XNA molecule may comprise an arabinonucleotide, an arabinonucleic acid (ANA) nucleotide, a 2′-Fluoro-arabinonucleic acid (FANA) nucleotide, a 2′-O-methyl ribonucleic acid (2′OMe) nucleotide, a 2′-O-methoxyethyl (MOE) nucleic acid nucleotide, a phosphorothioate 2′-O-methoxyethyl (PS-MOE) nucleotide, a phosphorodiamidate morpholino nucleotide, a locked nucleic acid (LNA) nucleotide, a P-alkyl phosphonate nucleic acid (phNA) nucleotide, a threose nucleic acid (TNA) nucleotide, a hexitol nucleic acid (HNA) nucleotide, a 2′ hydroxy-hexitol (AtNA) nucleotide, a cyclohexene nucleic acid (CeNA) nucelotide, or a 3′ deoxi-DNA (2′-5′) nucleotide.

The nucleic acid polymerase may comprise an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide. The amino acid sequence of the nucleic acid polymerase may comprise one or more, or all, of the following mutations: V93Q, D141A, E143A, and A485L. In particular, the polymerase may be TGK, TGLLK, 2M, Bst, RT521, 6G12, 6G12521, C7, PGLVV, PGLVVWA, D4K, or a variant thereof.

After step ii) a), the method may comprise cleaving the first nucleic acid and linearizing the bridge. The method may further comprises re-contacting the linearized product with the nucleic acid polymerase under conditions suitable for polymerisation.

In another aspect of the invention, there is provided a method of displaying a non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for polymerisation, wherein
   the primer for polymerisation is immobilised on the substrate such a bridge is formed during polymerisation, and
   the product of the polymerisation is a chain of non-DNA nucleotides that is immobilised on the substrate via the primer;
   b) cleaving the first nucleic acid and linearizing the bridge; and
   c) contacting the linearized product of step b) with a polymerase under conditions suitable for polymerisation; and
iii) removing the first nucleic acid to result in display of the second nucleic acid on the substrate.

The second nucleic acid may be an RNA molecule. The bridge may be denatured by temperature.

The first nucleic acid may be cleaved with formamidopyrimidine DNA glycosylase (Fpg) at an 8-oxoguanine site. A third nucleic acid may be annealed to the first nucleic acid at the 8-oxoguanine site before cleavage with Fpg.

Step ii) a) may comprise at least 5, 10, 12, 15, 20, or 25 cycles of bridged polymerisation. The first nucleic acid may be removed in step iii) by contacting the first nucleic acid with a denaturation reagent. The denaturation reagent may be a buffer comprising: 1-500 mM NaOH and 0-20 mM EDTA; or 100 mM NaOH and 5 mM EDTA.

In an embodiment, the second nucleic acid is an RNA molecule and encodes a polypeptide, and the method further comprises: iv) contacting the second nucleic acid with a ribosome under conditions suitable for translation of the encoded polypeptide. The conditions of step iv) may comprise trimethylamine N-oxide (TMAO). The TMAO may be at a concentration of 0.05-1.5 M, 0.05-1.2M, or 4 M.

In an aspect of the invention, there is provided a method of displaying a polypeptide on a substrate, comprising:
i) providing a first nucleic acid comprising an antisense sequence encoding a single-chain variable fragment (scFv), wherein the first nucleic acid is immobilised on a substrate, and wherein the first nucleic acid is oriented such that the 5′ end is proximal and the 3′ end is distal to the point of immobilisation:
ii) generating a second nucleic acid that is complementary to the first nucleic acid, wherein the generation of the second nucleic acid comprises:
   contacting the first nucleic acid with a nucleic acid polymerase under conditions suitable for RNA polymerisation, wherein
   the primer for polymerisation is immobilised on the substrate such a bridge is formed during polymerisation, and
   the product of the polymerisation is a chain of RNA nucleotides that is immobilised on the substrate via the primer:
iii) removing the first nucleic acid to result in display of the second nucleic acid on the substrate; and
iv) contacting the second nucleic acid with a ribosome under conditions suitable for translation of the encoded scFv, wherein the conditions of step iv) comprise trimethylamine N-oxide (TMAO).

The ribosome-polypeptide complex may be stabilised by the application of a ribosome display buffer. The ribosome display buffer may comprise a magnesium concentration which is: greater than 7 mM MgCl₂; or equivalent to 8, 9, 10, 15, 20, 25, 30, 40, 50, 60, 70, 80, 90, or 100 mM MgCl₂ or MgAc: or equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgCl₂; or equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgAc.

In an embodiment, the second nucleic acid is an RNA molecule and a plurality of first nucleic acids encoding a plurality of polypeptides are provided in step i), such that a display library is created by the method. The encoded polypeptide may be an antibody fragment or an enzyme. The encoded polypeptide may be a single-chain variable fragment (scFv), a peptide, a fibronectin type III domain (FN3 domain), a single-domain antibody (sdAb, also known as a nanobody), an affibody, a darpin, a fynomer, an OBody, or an avimer.

The first nucleic acid immobilised on the substrate as provided in step i) may be generated by:
1) providing a template nucleic acid:
2) hybridising the template nucleic acid to a primer immobilised to a substrate:
3) contacting the hybridised template nucleic acid with a polymerase under conditions suitable for the extension of the immobilised primer to synthesise the first nucleic acid which is a chain of nucleotides that are complementary to the template:
4) performing bridge amplification of the first nucleic acid to generate clusters of the first nucleic acid; and
5) sequencing at least a part of the first nucleic acid.

In an embodiment, the bridge amplification: comprises 32-35 amplification cycles, has an extension time of 60-120 seconds per cycle, comprises the use of an amplification buffer comprising Mg at a concentration equivalent to 2-6 mM of MgSO₄, and/or comprises the use of a denaturation buffer comprising 95-99.9% Formamide, optionally 1-10 mM NaOH, and optionally 1-5 mM EDTA. In a particular embodiment, the bridge amplification: comprises 32 amplification cycles, has an extension time of 60 seconds per cycle, comprises the use of an amplification buffer comprising Mg at a concentration equivalent to 6 mM of MgSO₄, and/or comprises the use of a denaturation buffer comprising 98% Formamide, 10 mM NaOH, and 1 mM EDTA.

In an aspect of the invention, there is provided a method of preparing clusters of substrate-bound nucleic acids, comprising:
1) providing a template nucleic acid:
2) hybridising the template nucleic acid to a primer immobilised to a substrate:
3) contacting the hybridised template nucleic acid with a polymerase under conditions suitable for the extension of the immobilised primer to synthesise the first nucleic acid which is a chain of nucleotides that are complementary to the template; and
4) performing bridge amplification of the first nucleic acid to generate clusters of the first nucleic acid, wherein the bridge amplification is carried out for 32-35 amplification cycles, has an extension time of 60-120 seconds per cycle, comprises the use of an amplification buffer comprising Mg at a concentration equivalent to 2-6 mM of MgSO₄, and comprises the use of a denaturation buffer comprising 95-99.9% Formamide, optionally 1-10 mM NaOH, and optionally 1-5 mM EDTA.

In an embodiment, the bridge amplification: comprises 32 amplification cycles, has an extension time of 60 seconds per cycle, comprises the use of an amplification buffer comprising Mg at a concentration equivalent to 6 mM of MgSO₄, and/or comprises the use of a denaturation buffer comprising 98% Formamide, 10 mM NaOH, and 1 mM EDTA.

In an aspect of the invention, there is provided a substrate displaying a non-DNA nucleic acid molecule which is obtained or obtainable by the methods disclosed herein. In an aspect of the invention, there is provided a substrate displaying an RNA molecule which is obtained or obtainable by the methods disclosed herein. In an aspect of the invention, there is provided a substrate displaying an XNA molecule which is obtained or obtainable by the methods disclosed herein. In an aspect of the invention, there is provided a substrate displaying a polypeptide molecule which is obtained or obtainable by the methods disclosed herein.

In an aspect of the invention, there is provided use of a nucleic acid polymerase to extend a DNA primer immobilised on a substrate to synthesise a non-DNA nucleic acid molecule that is complementary to a single-stranded nucleic acid template. The nucleic acid polymerase may comprise an amino acid sequence having at least 36% similarity or identity to the amino acid sequence of SEQ ID NO: 1 and comprises a Y409 and an E664 mutation, and wherein an RNA molecule is polymerised that is complementary to the nucleic acid template. The nucleic acid polymerase may comprise a sequence that has 80%, 90%, 95%, 99%, or 100% identity to the amino acid sequence of SEQ ID NO: 3, and residues 93, 141, 143, 409, 485, and 664 are invariant. The nucleic acid polymerase may comprise an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide. The amino acid sequence of the nucleic acid polymerase may comprise one or more, or all, of the following mutations: V93Q, D141A, E143A, and A485L. In particular, the polymerase may be TGK, TGLLK, 2M, Bst, RT521, 6G12, 6G12521, C7, PGLVV, PGLVVWA, D4K, or a variant thereof.

In an aspect of the invention, there is provided a method of screening a substrate displaying a plurality of biomolecules, wherein the substrate is any as disclosed herein, and wherein the biomolecules form a library.

In another aspect of the invention, there is provided a method of displaying a non-DNA nucleic acid molecule or a polypeptide, as disclosed herein, where the method further comprises screening the displayed non-DNA nucleic acid molecule or polypeptide molecule.

The screening disclosed herein may comprise measuring the affinity for a ligand or a target molecule, or measuring an enzymatic function, of the displayed biomolecules, non-DNA nucleic acid molecule, or polypeptide molecule.

### BRIEF DESCRIPTION OF THE DRAWINGS

FIG. 1 illustrates an exemplary process of displaying clusters of polypeptides on a substrate according to the invention. This illustration covers the process of A) generating clusters of DNA molecules and obtaining the position and sequence information. In an aspect, the method of the invention relates to an improved method of generating clusters, as disclosed herein. The improved method is particularly suitable for use with the display of long polypeptides in excess of 300 amino acids. B) converting clusters of DNA into clusters of RNA using an engineered polymerase and a linearisation step which releases torque built up during bridged RNA synthesis, and enables full length synthesis of long RNA molecules (>1.2 kb). C) in vitro translation and ribosome display of polypeptides encoded within the clusters of RNA, according to the described construct design, and the process of measuring binding affinities to the displayed polypeptides. D) downstream analysis of the sequencing and binding data.

FIG. 2 illustrates an example wherein a library of RNA molecules encoding single-chain variable fragments (scFvs) were produced on a flow cell. The upper panel, “Without torque release”, involved 12 cycles of RNA synthesis with TGK polymerase followed by linearization of the DNA template with Fpg. The lower panel, “With torque release”, involved 12 cycles of RNA synthesis with TGK polymerase followed by two cycles of linearization of the DNA template with Fpg and further RNA synthesis with TGK polymerase. The images show the level of binding of a fluorescent oligonucleotide probe to the 3′ end of the RNA. The amount of RNA synthesis achieved by the methods for producing the upper panel is incomplete and much higher levels are achieved by methods of the lower panel.

FIG. 3 illustrates an experiment to determine the effects of magnesium concentration in ribosome display buffers. Some prior art methods are limited to magnesium concentrations that are equivalent to 7 mM of MgCl₂ or lower, because higher concentrations would denature the stalled RNAP:RNA complexes. The presently disclosed methods may be used with much higher concentrations of magnesium. FIG. 3 shows the level of Her2 binding (100 nM Her2-biotin and 100 nM AF532-strepavidin) in low magnesium conditions vs high magnesium conditions. The high magnesium conditions led to a five-fold improvement in binding and display efficiency.

FIG. 4. The deep screening workflow. 1) Deep screening begins with library preparation, which involves the addition of 5′ and 3′ un-translated region (UTR) that flank the library protein coding region. The assembled library is then clustered and the N28 UMI is sequenced on a HiSeq 2500, which reports the UMI sequence and its physical x-y coordinates on the flow cell. 2) The sequenced flow cell is subsequently used for deep screening by converting the clusters of DNA into clusters of RNA and removing the DNA template. The RNA clusters are labelled with an Atto647N labelled oligo before being translated into proteins and tethered to the RNA via ribosome display. Following display an on-chip binding assay is conducted by equilibrium binding of an increasing concentration of biotinylated antigen and AF532 labelled streptavidin, before performing a kinetic dissociation. 3) If the binding assay reports hits within the library, a sequencing run is performed on a fresh flow cell to sequence the UMI and CDRs with internal sequencing primers. CDRs are then paired with binding data using the common UMIs between the two experiments. 4) Paired CDR: binding data is analysed for hits and or a machine learning model is trained to predict hits. This can either be used to generate libraries for subsequent rounds of deep screening or 5) short list hit candidates for characterisation in an appropriate format.

FIG. 5. A) Workflow of selections. B) Library statistics. C) Abundance vs. deep screening integrated binding intensity at 300 nM HEL for the R3 MACS and R3 FACS libraries. The library mean intensity is shown as a grey dashed line and a solid green line shows the hit threshold of 2× the library background. Spearman rank correlation constants of 0.361 and 0.442 respectively show a poor correlation between abundance and deep screening binding intensities. D) Hit candidates for characterization, showing the library construct structure and ID, where M1-23 are derived from the R3 MACS library, C1-8 were identified from colony picking the R3 MACS library and F1-10 were derived from the R3 FACS library. The abundance, CDR sequences, a deep screening fitted equilibrium binding K_{D} and Octet fitted kinetic K_{D}s are also shown. The sequences shown are, in order of first appearance, SEQ ID NOs: 41-150. E) Deep screening equilibrium binding and kinetic dissociation curves for clones M5, M6, M14 and M15. F) Octet kinetics at 50 nM of the same 4 clones against a HEL-biotin loaded streptavidin tip. G) Octet K_{D}s plotted against deep screening binding intensities at 300 nM HEL for all characterised clones, revealing a Spearman rank correlation constant of −0.697.

FIG. 6. A) Overview of the direct affinity maturation experiment. B) Library statistics of the unselected L1L3 library from deep screening. C) Background normalized deep screening intensities at 0, 100 pM and 1 nM of huIL-7, sorted by intensity (rank). D) IL70001 and top 19 clones, showing VL1 and VL3 sequences, raw deep screening intensities at 333 pM huIL-7, Octet fitted K_{D}s and IL7R IC₅₀s. The sequences shown are, in order of first appearance, SEQ ID NOs: 151-176. E) Octet kinetics at 50 nM of IL70001, IL70100, IL70102 and IL70105 Fabs against a huIL-7 loaded streptavidin tip. F) Deep screening mean intensities of the top 19 clones at 333 pM huIL-7 plotted against fitted Octet K_{D}s. Error bars are standard error of the mean (SEM). Grey vertical line is showing the mean library intensity at 333 pM huIL-7. G) TF-1 STAT5 IL7R alpha+gamma luciferase inhibition assay, showing IL70001, IL70100, IL70102 and IL70105 as a representative range of the assay. All inhibition assay curves are shown in FIG. 15. Error bars are standard deviation, n=2. H) Plotting BLI/Octet fitted K_{D}s against IC₅₀ reveals a strong, non-linear correlation between affinity and inhibition (p=0.956, R2=0.901 for fitting log (v)=m*log (x)+c (grey line)).

FIG. 7 illustrates an experiment displaying a library of characterised anti-Her2 single chain antibodies (scFvs) and measuring equilibrium binding affinities and kinetic dissociation rates. A) Construct design showing CDR sequences (VH3, VL1 and VL3) and binding affinities of clones G98A, C6.5, ML3-9, H3B1 and BID2+A1. The sequences shown are, in order of first appearance, SEQ ID NOs: 177-184. B) Flow cell images during equilibrium binding and kinetic dissociation. Illustrated are raw images of the flow cell at increasing concentrations of Her2-biotin and 100 nM AF532-streptavidin and raw images of the flow cell after an increasing amount of time with binding buffer injected over the flow cell at 100 μl/minute. Images are set to the same min/max threshold of 100/1000. C) Curve fits to equilibrium binding and kinetic dissociation data, showing clones from A) and the addition of Herceptin (trastuzumab). This illustrates the processed image data and median integrated binding signal plotted per concentration of Her2-biotin. Equilibrium binding curves are fitted to the data, and error bars are shown as standard error of the mean (SEM). Given the large number of clusters observed per clone, the SEM is not visible on the plot. The second graph illustrates the processed image data and median integrated binding signal plotted over time of washing. A two-phase, heterogeneous dissociation rate is fitted to the data, with error bars shown as SEM.

FIG. 8. Affinity maturation of G98A. A) Construct schematic of G98A, showing its CDR H3 sequence and a depiction of how the six scanning window NNS sub-libraries are structured (the sequence is SEQ ID NO: 177. B) Experiment statistics showing 159.8M clusters in the deep screening component, which resulted in 297 k unique barcodes with 12 replicates and 236 k unique CDR VH3 protein sequences. The sequences shown are SEQ ID NOs: 177, 180, 185-187. C) PCA plot showing all 236 k VH3 protein sequences projected into two dimensions and coloured by mean fluorescent intensity at 100 nM of Her2. A red dot shows the position of G98A wild-type relative to the library. D) CDR H3 sequences of G98A, ML3-9 and three of the top scoring clones identified by deep screening. Next to the sequences are the deep screening (DS) fitted equilibrium binding K_{D}s and binding K_{D}s identified via Octet. E) Deep screening equilibrium binding and kinetic dissociation curves showing G98A, ML3-9 and three of the top scoring clones. Error bars are SEM. F) Octet kinetics of G98A, ML3-9 and three of the top scoring clones at 20 nM of each clone on a Her2 loaded octet tip.

FIG. 9. Machine learning guided antibody engineering. A) The workflow used for in silico mutagenesis of three anti-Her2 seed sequences and selection of 13,121 random mutations and 11,916 ML guided mutations prior to a second round of deep screening. The sequences shown are SEQ ID NOs: 188-192. B) Evaluation of the selected ML and random mutations at the 5 minute wash condition, which reveals a substantial, 5-fold, shift in the binding distribution of the ML mutants relative to making random mutations. C) Flow cell derived equilibrium binding and kinetic dissociation curves of G98A, Her20006, 13 and 19 as scFvs. Error bars are SEM. D) Octet derived binding kinetics of a Her2 loaded tip and purified Fabs at a concentration of 20 nM.

FIG. 10. Deep screening derived equilibrium binding and kinetic dissociation curves for the anti-HEL nanobody clones selected from the MACS library for characterisation. Each concentration condition within curve represents at least 12 measurements from a deep screening experiment. Error bars are SEM. We report an equilibrium K_{D}, area under the curve (AUC) for the equilibrium binding and two dissociation rates for a biphasic dissociation model, as well as an AUC.

FIG. 11. Deep screening derived equilibrium binding and kinetic dissociation curves for the anti-HEL nanobody clones selected by picking 96 colonies from the R3 MACS output and clones selected from the R3 FACS library screen. Each concentration condition within curve represents at least 12 measurements from a deep screening experiment. Error bars are SEM. We report an equilibrium Kp, area under the curve (AUC) for the equilibrium binding and two dissociation rates for a biphasic dissociation model, as well as an AUC.

FIG. 12. Octet measured association and dissociation kinetics for the anti-HEL nanobody clones selected from the MACS (M1-M23) library, where nanobody clones were bound at 50 nM to a HEL-biotin loaded streptavidin tip.

FIG. 13. Octet measured association and dissociation kinetics for the anti-HEL nanobody clones selected from picking 96 colonies (C1-C8) and from the MACS library screen (M1-M10), where nanobody clones were bound at 50 nM to a HEL-biotin loaded streptavidin tip.

FIG. 14. Octet measured association and dissociation kinetics for the anti-IL7 scFv clones selected for characterisation. Where each clone was converted from scFv to Fab, expressed, purified, and normalised to 50 nM. Fabs were then bound to a streptavidin tip preloaded with huIL7-biotin. A 1:1 model was fit to all clones, except for IL70001.

FIG. 15. TF-1 STAT5 IL7 receptor (IL7R) alpha+gamma luciferase inhibition assay, showing IL7R signalling luminescence plotted against the log molar concentration of all characterised clones individually. Error bars are standard deviation, n=2.

FIG. 16. TF-1 STAT5 IL7 receptor (IL7R) alpha+gamma luciferase inhibition assay, showing IL7R signalling luminescence plotted against the log molar concentration of all characterised clones. Error bars are standard deviation, n=2.

FIG. 17. Deep screening derived equilibrium binding and kinetic dissociation curves for the anti-Her2 scFvs selected for characterisation. Each concentration condition within curve represents at least 12 measurements from either “Her2affmat” (G98A to HER20011) or “Her2 ML vs. Random” (HER20012 to HER20026) deep screening experiments. Error bars are SEM. We report an equilibrium Kp, area under the curve (AUC) for the equilibrium binding and two dissociation rates for a biphasic dissociation model, as well as an AUC.

FIG. 18. Octet measured association and dissociation kinetics for the anti-Her2 scFv clones selected for characterisation. Each clone was converted from scFv to Fab, expressed, purified, and normalised to 20 nM. Fabs were then bound to a streptavidin tip preloaded with Her2-biotin. A 1:1 model was fit to all clones, except for G98A.

FIG. 19 demonstrates the successful display and a functional fluorescent assay of FANA polymers and 2′OMe-RNA polymers on a substrate.

FIG. 20 demonstrates the successful display and a functional fluorescent assay of peptides, fibronectin type III (FN3) scaffolds, nanobodies, and scFvs on a substrate.

FIG. 21 illustrates exemplary XNAs that may be displayed by the methods of the invention.

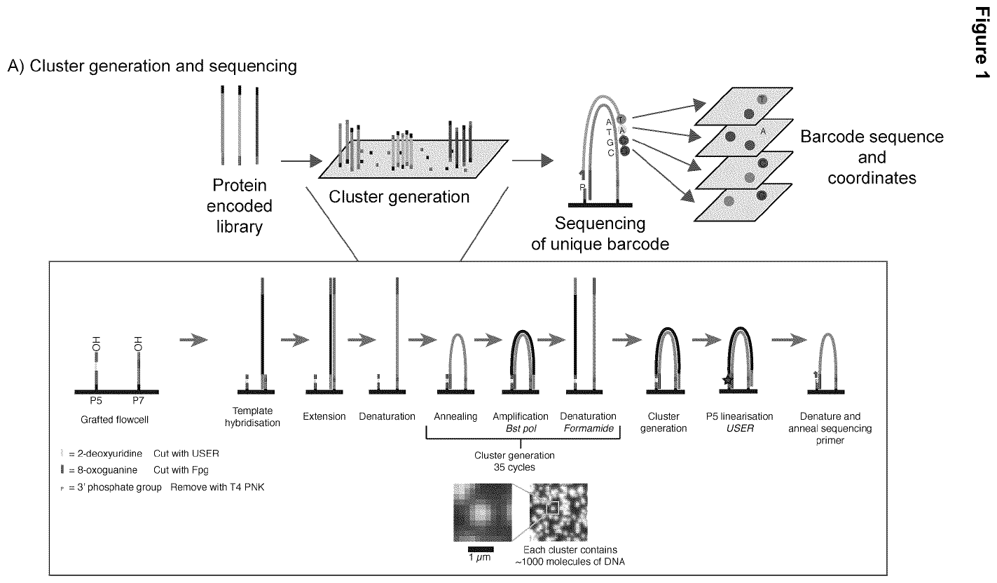

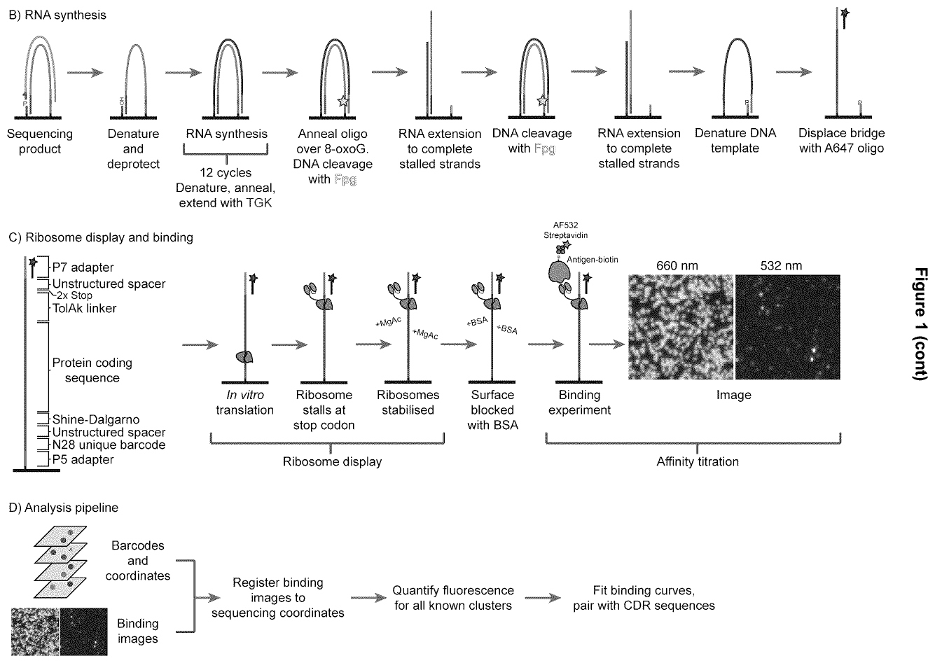

### DETAILED DESCRIPTION

Techniques that allow the display of biomolecules on substrates are important for enabling downstream analysis, such as high-throughput screening. The inventors provide herein techniques that allow for the display of non-DNA nucleic acids on substrates.

In an aspect, the present invention makes use of polymerases that are capable of synthesising non-DNA nucleic acids from DNA primers to generate biomolecules that are immobilised to a substrate.

Thus, in an aspect of the present invention, there is provided a method of displaying a non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for polymerisation, wherein
   the primer for polymerisation is a DNA primer immobilised on the substrate such a bridge is formed during polymerisation,
   the product of the polymerisation is a chain of non-DNA nucleotides that is immobilised on the substrate via the primer, and
the nucleic acid polymerase is a polymerase capable of acting upon a DNA primer to synthesise a non-DNA nucleic acid molecule that is complementary to a single-stranded nucleic acid template; and
iii) removing the first nucleic acid to result in display of the second nucleic acid on the substrate.

The resultant second nucleic acid is a single-stranded non-DNA nucleic acid molecule displayed on the substrate. Conditions suitable for the polymerisation of non-DNA nucleic acids are known in the art and include, for instance, the provision of the appropriate nucleotides, such as RNA or XNA nucleotides.

In some embodiments, the non-DNA nucleic acids are XNA molecules. XNA molecules comprise nucleotide chains with a non-naturally occurring sugar backbone, non-naturally occurring nucleobases, non-naturally occurring phosphodiester linkages, non-naturally occurring linkages, or any combination thereof. The XNAs may be any that can be polymerised by a polymerase capable of acting upon a DNA primer to synthesise an XNA molecule. In particular, the XNAs may be any naturally modified or any non-natural nucleic acid for which a natural or engineered polymerase can synthesise a polynucleotide from a DNA template using a DNA primer. Suitable polymerases are discussed herein.

For instance, the XNA molecule may comprise arabinonucleotides, which are structural analogues of deoxynucleotides and differ only by the presence of a β-hydroxyl at the 2′ position of the sugar moiety. The arabino nucleotide molecule may be an arabinonucleic acid (ANA) molecule or a 2′-Fluoro-arabinonucleic acid (FANA) molecule. In other embodiments, the XNA molecule may be a 2′-O-methyl ribonucleic acid (2′OMe) molecule, a 2′-O-methoxyethyl (MOE) nucleotide, a phosphorothioate 2′-O-methoxyethyl (PS-MOE) nucleotide, a phosphorodiamidate morpholino oligonucleotide (PMO), or a combination thereof. Alternatively, the XNAs may be β-alkyl phosphonate nucleic acid (phNA). In phNAs, the non-bridging oxygen of the canonical phosphodiester linkage is replaced by an uncharged alkyl substituent, specifically a methyl (Met) or ethyl (Et)) group. In other embodiments, the XNA molecule may be a threose nucleic acid (TNA), a hexitol nucleic acid (HNA), a 2′ hydroxy-hexitol (AtNA), a cyclohexene nucleic acid (CeNA), a locked nucleic acid (LNA), or 3′ deoxi-DNA (2′-5′).

In some embodiments, the non-DNA nucleic acids are RNA molecules. The RNA molecules may include natural and unnatural modifications, such as m6A, 5-ethinyl-U, diaminopurine, phosphorothioate, 2′Fluoro, 2′N₃, 2′NH₂, 3′O-methyl, and unnatural base-pair derivatives. In some embodiments, the RNA molecules are unmodified.

The following table lists examples of RNAs and XNAs that may be displayed according to methods of the invention.

| Chemistry | NTPs | Exemplary Polymerase |
|---|---|---|
| RNA* | NTPs* | TGK |
| 2′F-RNA | 2′F-NTPs | TGK |
| 2′N3-RNA | 2′N3-NTPs | TGK |
| 2′NH2-RNA | 2′NH2-NTPs | TGK, TGLLK |
| 2′O-methyl-RNA | 2′OMe-NTPs | TGLLK, 2M |
| 3′ deoxi-DNA (2′-5′) | 3′-dNTPs | TGLLK |
| 3′O-methyl-RNA | 3′OMe-dNTPs | TGLLK |
| TNA | tNTPs | Bst, RT521 |
| 2′-O-2-methoxyethyl-RNA | MOE-NTPs | 2M |
| Phosphorothioate 2′-O-2- | PS-MOE-NTPs | 2M |
| methoxyethyl-RNA | | |
| HNA | hNTPs | 6G12, 6G12521 |
| AtNA | atNTPs | 6G12521 |
| CeNa | ceNTPs | C7, 6G12521 |
| LNA | INTPs | C7, 2M, 6G12521 |
| phNA | phNTPs | PGLVV, PGLVVWA |
| PMOs, P-alkyl-moNAs | mo-NTPs | PGLVV, PGLVVWA |
| FANA | fNTPs | D4K |
| ANA | aNTPs | D4K |
| PS-RNA | psNTPs | TGK |
| (phosphorothioate RNA) | | |

*Including RNA comprising modifications such as m6A, 5-ethinyl-U, diaminopurine, unnatural base-pairs etc.

The above table lists exemplary polymerases for making nucleic acid polymers. The exemplary polymerases are discussed further herein.

In embodiments of displaying RNA on a substrate, the first nucleic acid may encode a polypeptide. For instance, the first nucleic acid may include an antisense sequence that may act as a template for an RNA molecule capable of being translated into a protein.

The first nucleic acid of step i), may be a nucleic acid that is part of a cluster that has been generated on a substrate. For instance, the nucleic acid may be a DNA molecule with a first adapter at one end and a second adapter at the other end, which has been bound to the substrate via an immobilised primer capable of hybridising to one of the adapters. The nucleic acid may then have been amplified into a cluster, for instance via bridge amplification making use of the aforementioned primer and a second immobilised primer capable of hybridising to the other adapter. Such methods for generating clusters of DNA molecules are known in the art, and the invention encompasses the use of any such method. Particularly preferred methods are disclosed herein.

A cluster of nucleic acids is a term of the art and relates to a group of immobilised nucleic acid molecules that are in close proximity. The close proximity is commonly because the cluster is generated by amplification from a single parent molecule, and hence the cluster is of nucleic acids comprising the same sequence. Examples of techniques for forming clusters include bridge amplification and kinetic exclusion exponential amplification.

The substrate may be a solid surface such as a surface of a flow cell, a bead, a slide, or a membrane. In particular, the substrate may be a flow cell. The flow cell may be patterned or non-patterned. The substrate may comprise glass, quartz, silica, metal, ceramic, or plastic. The substrate surface may comprise a polyacrylamide matrix or coating.

As used herein, the term “flow cell” is intended to have the ordinary meaning in the art, in particular in the field of sequencing by synthesis. Exemplary flow cells include, but are not limited to, those used in a nucleic acid sequencing apparatus such as flow cells for the Genome AnalyzerR, MiSeq®, NextSeq®, HiSeq® or NovaSeq® platforms commercialised by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO 2014/142841 A1: U.S. Pat. App. Pub, No. 2010/0111768 A1 and U.S. Pat. No. 8,951,781.

At least a part of the first nucleic acid may have been sequenced before step i) of the method of displaying a non-DNA nucleic acid molecule on a substrate. For instance, at least one adapter may comprise a barcode sequence and said barcode may be sequenced. As such, the coordinates of each barcode sequence on the substrate may be known. Such techniques are known in the art.

Immobilisation to a substrate means that the nucleic acid is bound to the substrate even under conditions that would denature double-stranded nucleic acids. For instance, the nucleic acid may be covalently bound to the substrate. The nucleic acid may be immobilised on a polyacrylamide coated substrate.

The first nucleic acid is oriented such that the 5′ end is proximal and the 3′ end is distal to the point of immobilisation. Such arrangements may enable bridge amplification in combination with an immobilised primer. These arrangements are standard in the art.

A nucleic acid bridge is a term of the art and relates to a nucleic acid which is bound at both ends to a substrate. Usually, one end (e.g. the 5′ end) is immobilised to the substrate and the other end is bound via hybridisation to a complementary nucleic acid which is, itself, immobilised to the substrate. Bridge amplification takes place when the template is a bridge.

In embodiments of the invention, the immobilised first nucleic acid is contacted with a nucleic acid polymerase under conditions suitable for polymerisation. The primer for polymerisation is a DNA primer which is also immobilised on the substrate and, as such, a bridge is formed during polymerisation. A polymerase is used which is capable of acting upon the DNA primer to synthesise a non-DNA molecule that is complementary to the first nucleic acid. As such, the product of the polymerisation is a chain of non-DNA nucleotides that is immobilised on the substrate via the primer.

The DNA primer may comprise modified or non-DNA nucleotides. However, the DNA primer does not comprise RNA nucleotides at the 3′ terminus, and thus the polymerase is a polymerase that does not require an RNA primer. In particular, the methods of the invention are suitable for use with commercially available adapters/primers such as Illumina's P5 and P7 adapters, and the methods do not require the modification of said primers with ribonucleotides. For instance, 3Dpol from poliovirus is an RNA-dependent RNA polymerase that is not capable of acting upon a DNA primer.

Polymerases capable of acting upon DNA primers to synthesise XNA polymers are disclosed in publications such as Arangundy-Franklin et al. (Nature Chemistry volume 11, pages 533-542 (2019)), WO2011/135280, and WO2013/156786. As disclosed in these publications, mutations in the backbone of polymerases of the polB family, excluding viral polymerases, may render the polymerase capable of synthesising XNA polymers. In particular, the backbone may be of a polymerase from the Archaeal Thermococcus and/or Pyrococcus genera.

The polymerase may be a variant of the polymerase from T. gorgonarius mutated so as to allow the polymerisation of XNA molecules. The polymerase may comprise an amino acid sequence having at least 36%, 50%, 60%, 70%, 80%, 90%, 95%, 99%, or 100% similarity or identity to the amino acid sequence of SEQ ID NO: 1. The polymerase may comprise an amino acid sequence having at least 36%, 50%, 60%, 70%, 80%, 90%, 95%, 99%, or 100% similarity or identity to the amino acid sequence of SEQ ID NO: 1, comprising mutations that allow the polymerisation of RNA or XNAs.

The nucleic acid polymerase may be a polymerase which is capable of acting upon a DNA primer to synthesise an RNA molecule or an XNA molecule, such as 2′F-RNA, 2′N₃-RNA, 2′NH₂-RNA, or PS-RNA, that is complementary to a single-stranded nucleic acid template. Such polymerases include any polymerase capable of synthesising an RNA molecule or an XNA molecule as disclosed in WO2011/135280 or Cozens et al. (Cozens, Pinheiro, Vaisman, Woodgate, and Holliger, A short adaptive path from DNA to RNA polymerases, PNAS May 22, 2012 109 (21) 8067-8072: https://doi.org/10.1073/pnas.1120964109), each of which is herein incorporated by reference. For instance, the polymerase may be D4N, TNQ, TNK, or TGK as disclosed in said documents, or variants thereof. In particular, the polymerase may be TGK, or a variant thereof.

The polymerase may include mutations corresponding to Y409N or Y409G and E664K or E664Q (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera.

The polymerase may be a variant of the polymerase from T. gorgonarius (Tgo) comprising addition mutations to allow RNA polymerase activity. The sequence of wild type Tgo is shown below:

(SEQ ID NO: 1)
MILDTDYITEDGKPVIRIFKKENGEFKIDYDRNFEPYIYALLKDDSAIEDVKKITAERHGTTVRV
VRAEKVKKKFLGRPIEVWKLYFTHPQDVPAIRDKIKEHPAVVDIYEYDIPFAKRYLIDKGLIPME
GDEELKMLAFDIETLYHEGEEFAEGPILMISYADEEGARVITWKNIDLPYVDVVSTEKEMIKRFL
KVVKEKDPDVLITYNGDNFDFAYLKKRSEKLGVKFILGREGSEPKIQRMGDRFAVEVKGRIHFDL
YPVIRRTINLPTYTLEAVYEAIFGQPKEKVYAEEIAQAWETGEGLERVARYSMEDAKVTYELGKE
FFPMEAQLSRLVGQSLWDVSRSSTGNLVEWFLLRKAYERNELAPNKPDERELARRRESYAGGYVK
EPERGLWENIVYLDFRSLYPSIIITHNVSPDTLNREGCEEYDVAPQVGHKFCKDFPGFIPSLLGD
LLEERQKVKKKMKATIDPIEKKLLDYRQRAIKILANSFYGYYGYAKARWYCKECAESVTAWGRQY
IETTIREIEEKFGFKVLYADTDGFFATIPGADAETVKKKAKEFLDYINAKLPGLLELEYEGFYKR
GFFVTKKKYAVIDEEDKITTRGLEIVRRDWSEIAKETQARVLEAILKHGDVEEAVRIVKEVTEKL
SKYEVPPEKLVIYEQITRDLKDYKATGPHVAVAKRLAARGIKIRPGTVISYIVLKGSGRIGDRAI
PFDEFDPAKHKYDAEYYIENQVLPAVERILRAFGYRKEDLRYQKTRQVGLGAWLKPKT

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises a Y409 and a E664 mutation relative to the amino acid sequence of SEQ ID NO:1. In embodiments, the Y409 mutation is Y409N or Y409G and the E664 mutation is E664K or E664Q. In particular embodiments, the Y409 mutation and the E664 mutation are in the following combinations: i) Y409N and E664Q, ii) Y409N and E664k, or iii) Y409G and E664K. The amino acid sequence of the nucleic acid polymerase may further comprise one or more, or all, of the following mutations: A485L, V93Q, D141A, and E143A.

V93Q is a mutation known to disable uracil-stalling, D141A and E143A reduce 3′-5′ exonuclease function, and the “Therminator” mutation (A485L) is known to enhance the incorporation of unnatural substrates. The sequence of the Tgo polymerase comprising these mutations (henceforth termed TgoT) is shown below:

(SEQ ID NO: 2)
MILDTDYITEDGKPVIRIFKKENGEFKIDYDRNFEPYIYALLKDDSAIEDVKKITAERHGTTVRV
VRAEKVKKKFLGRPIEVWKLYFTHPQDQPAIRDKIKEHPAVVDIYEYDIPFAKRYLIDKGLIPME
GDEELKMLAFAIATLYHEGEEFAEGPILMISYADEEGARVITWKNIDLPYVDVVSTEKEMIKREL
KVVKEKDPDVLITYNGDNFDFAYLKKRSEKLGVKFILGREGSEPKIQRMGDRFAVEVKGRIHFDL
YPVIRRTINLPTYTLEAVYEAIFGQPKEKVYAEEIAQAWETGEGLERVARYSMEDAKVTYELGKE
FFPMEAQLSRLVGQSLWDVSRSSTGNLVEWELLRKAYERNELAPNKPDERELARRRESYAGGYVK
EPERGLWENIVYLDFRSLYPSIIITHNVSPDTLNREGCEEYDVAPQVGHKFCKDFPGFIPSLLGD
LLEERQKVKKKMKATIDPIEKKLLDYRQRLIKILANSFYGYYGYAKARWYCKECAESVTAWGRQY
IETTIREIEEKFGFKVLYADTDGFFATIPGADAETVKKKAKEFLDYINAKLPGLLELEYEGFYKR
GFFVTKKKYAVIDEEDKITTRGLEIVRRDWSEIAKETQARVLEAILKHGDVEEAVRIVKEVTEKL
SKYEVPPEKLVIYEQITRDLKDYKATGPHVAVAKRLAARGIKIRPGTVISYIVLKGSGRIGDRAI
PFDEFDPAKHKYDAEYYIENQVLPAVERILRAFGYRKEDLRYQKTRQVGLGAWLKPKT

The polymerase may be according to SEQ ID NO: 2 comprising the following mutations: i) Y409N and E664Q (TNQ), ii) Y409N and E664K (TNK), or iii) Y409G E664K (TGK).

In a preferred embodiment, the amino acid sequence of the nucleic acid polymerase comprises SEQ ID NO: 1 and the mutations V93Q, D141A, E143A, Y409G, A485L, and E664K (TGK), as shown below:

(SEQ ID NO: 3)
MILDTDYITEDGKPVIRIFKKENGEFKIDYDRNFEPYIYALLKDDSAIEDVKKITAERHGTTVRV
VRAEKVKKKFLGRPIEVWKLYFTHPQDQPAIRDKIKEHPAVVDIYEYDIPFAKRYLIDKGLIPME
GDEELKMLAFAIATLYHEGEEFAEGPILMISYADEEGARVITWKNIDLPYVDVVSTEKEMIKREL
KVVKEKDPDVLITYNGDNFDFAYLKKRSEKLGVKFILGREGSEPKIQRMGDRFAVEVKGRIHFDL
YPVIRRTINLPTYTLEAVYEAIFGQPKEKVYAEEIAQAWETGEGLERVARYSMEDAKVTYELGKE
FFPMEAQLSRLVGQSLWDVSRSSTGNLVEWELLRKAYERNELAPNKPDERELARRRESYAGGYVK
EPERGLWENIVYLDFRSLGPSIIITHNVSPDTLNREGCEEYDVAPQVGHKFCKDFPGFIPSLLGD
LLEERQKVKKKMKATIDPIEKKLLDYRQRLIKILANSFYGYYGYAKARWYCKECAESVTAWGRQY
IETTIREIEEKFGFKVLYADTDGFFATIPGADAETVKKKAKEFLDYINAKLPGLLELEYEGFYKR
GFFVTKKKYAVIDEEDKITTRGLEIVRRDWSEIAKETQARVLEAILKHGDVEEAVRIVKEVTEKL
SKYEVPPEKLVIYKQITRDLKDYKATGPHVAVAKRLAARGIKIRPGTVISYIVLKGSGRIGDRAI
PFDEFDPAKHKYDAEYYIENQVLPAVERILRAFGYRKEDLRYQKTRQVGLGAWLKPKT.

The polymerase may be of an amino acid sequence having at least 36%, 50%, 60%, 70%, 80%, 90%, 95%, 99%, or 100% similarity or identity to the amino acid sequence of SEQ ID NO: 3, wherein residues 93, 141, 143, 409, 485, and 664 are invariant (i.e. the mutations V93Q, D141A, E143A, Y409G, A485L, and E664K are maintained).

In a certain embodiment, there is provided a method of displaying an RNA molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for RNA polymerisation, wherein
   the primer for polymerisation is a DNA primer immobilised on the substrate such a bridge is formed during polymerisation,
   the product of the polymerisation is a chain of RNA nucleotides that is immobilised on the substrate via the primer, and
   the nucleic acid polymerase is a polymerase capable of acting upon a DNA primer to synthesise an RNA molecule that is complementary to a single-stranded nucleic acid template, and comprises a sequence that has at least 80%, 90%, 95%, 99%, or 100% identity to the amino acid sequence of SEQ ID NO: 3, wherein residues 93, 141, 143, 409, 485, and 664 are invariant; and
iii) removing the first nucleic acid to result in display of the second nucleic acid on the substrate. In this embodiment, the second nucleic acid is an RNA polymer.

The nucleic acid polymerase may be a polymerase which is capable of acting upon a DNA primer to synthesise an XNA, for instance an arabino nucleotide polymer such as an ANA molecule or a FANA molecule, that is complementary to a single-stranded nucleic acid template. Such polymerases include any polymerase capable of synthesising an arabino nucleotide polymer molecule as disclosed in WO2013/156786 A1 (incorporated by reference herein). In a particular embodiment, the polymerase may be the D4YK polymerase as disclosed in WO2013/156786 A1. Such polymerases include any polymerase capable of synthesising said polymers as disclosed in Pinheiro et al. (Synthetic genetic polymers capable of heredity and evolution: Science. 2012 Apr. 20; 336 (6079): 341-344). For instance, the polymerase may be D4K as disclosed in said document, or variants thereof.

The polymerase may include mutations corresponding to P657T, E658Q, K659H, Y663H, E664K, D669A, K671N, and T676I (described relative to SEQ ID NO: 1) in the backbone of any polymerase from the polB family. The polymerase may further comprise L403P. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The L403P mutation is a further useful mutation in the A-motif of the polymerase. This has the advantage of assisting polymerisation and can help make longer polymers. This can improve polymerisation of arabino nucleotides by 3- or 4-fold, or even more. In some applications the improvement can be as high as 10-fold.

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations P657T, E658Q, K659H, Y663H, E664K, D669A, K67IN, and T676I, and optionally L403P, relative to the amino acid sequence of SEQ ID NO: 1.

The amino acid sequence of the nucleic acid polymerase may further comprise one or more, or all, of the following mutations: V93Q, D141A, E143A, and A485L. The mutations V93Q, D141A, E143A, and A485L are discussed herein elsewhere.

In a particular embodiment, nucleic acid polymerase which is capable of acting upon a DNA primer to synthesise an arabino nucleotide polymer, may be of an amino acid sequence having at least 80%, 90%, 95%, 99%, or 100% similarity or identity to the amino acid sequence of SEQ ID NO: 1, wherein said amino acid sequence comprises the mutations P657T, E658Q, K659H, Y663H, E664K, D669A, K67IN, T676I, V93Q, D141A, E143A, L403P, and A485L relative to the amino acid sequence of SEQ ID NO: 1.

In a particular embodiment, the nucleic acid polymerase which is capable of acting upon a DNA primer to synthesise an arabino nucleotide polymer, may comprise or may be of the following amino acid sequence:

MILDTDYITEDGKPVIRIFKKENGEFKIDYDRNFEPYIYALLKDDSAIEDVKKITAERHGTTVRV
VRAEKVKKKFLGRPIEVWKLYFTHPQDQPAIRDKIKEHPAVVDIYEYDIPFAKRYLIDKGLIPME
GDEELKMLAFAIATLYHEGEEFAEGPILMISYADEEGARVITWKNIDLPYVDVVSTEKEMIKRFL
KVVKEKDPDVLITYNGDNFDFAYLKKRSEKLGVKFILGREGSEPKIQRMGDRFAVEVKGRIHFDL
YPVIRRTINLPTYTLEAVYEAIFGQPKEKVYAEEIAQAWETGEGLERVARYSMEDAKVTYELGKE
FFPMEAQLSRLVGQSLWDVSRSSTGNLVEWELLRKAYERNELAPNKPDERELARRRESYAGGYVK
EPERGLWENIVYPDFRSLYPSIIITHNVSPDTLNREGCEEYDVAPQVGHKFCKDFPGFIPSLLGD
LLEERQKVKKKMKATIDPIEKKLLDYRQRLIKILANSFYGYYGYAKARWYCKECAESVTAWGRQY
IETTIREIEEKFGFKVLYADTDGFFATIPGADAETVKKKAKEFLDYINAKLPGLLELEYEGFYKR
GFFVTKKKYAVIDEEDKITTRGLEIVRRDWSEIAKETQARVLEAILKHGDVEEAVRIVKEVTEKL
SKYEVPTQHLVIHKQITRALNDYKAIGPHVAVAKRLAARGIKIRPGTVISYIVLKGSGRIGDRAI
PFDEFDPAKHKYDAEYYIENQVLPAVERILRAFGYRKEDLRYQKTRQVGLGAWLKPKT (SEQ
ID NO: 4; also known as D4YK or D4K).

The polymerase may be of an amino acid sequence having at least 36%, 50%, 60%, 70%, 80%, 90%, 95%, 99%, or 100% similarity or identity to the amino acid sequence of SEQ ID NO: 4, wherein residues 93, 141, 143, 403, 485, 657, 658, 659, 663, 664, 669, 671, and 676 are invariant (i.e. the mutations V93Q, D141A, E143A, L403P, A485L, P657T, E658Q, K659H, Y663H, E664K, D669A, K671N, and T676I, are maintained).

In a certain embodiment, there is provided a method of displaying an arabino nucleotide polymer on a substrate, comprising:
i) providing a first nucleic acid immobilised on a substrate, and wherein the first nucleic acid is oriented such that the 5′ end is proximal and the 3′ end is distal to the point of immobilisation:
ii) generating a second nucleic acid that is complementary to the first nucleic acid, wherein the generation of the second nucleic acid comprises:
   a) contacting the first nucleic acid with a nucleic acid polymerase under conditions suitable for arabino nucleotide polymerisation, wherein
   the primer for polymerisation is a DNA primer immobilised on the substrate such a bridge is formed during polymerisation,
   the product of the polymerisation is a chain of arabino nucleotides that is immobilised on the substrate via the primer, and
   the nucleic acid polymerase is a polymerase capable of acting upon a DNA primer to synthesise an arabino nucleotide polymer that is complementary to a single-stranded nucleic acid template, and comprises a sequence that has at least 80%, 90%, 95%, 99%, or 100% identity to the amino acid sequence of SEQ ID NO: 4, wherein residues 93, 141, 143, 403, 485, 657, 658, 659, 663, 664, 669, 671, and 676 are invariant; and
iii) removing the first nucleic acid to result in display of the second nucleic acid on the substrate. In this embodiment, the second nucleic acid is a single-stranded arabino nucleotide polymer displayed on the substrate. In a particular embodiment, the arabino nucleotide polymer displayed on the substrate is an ANA molecule or a FANA molecule.

The nucleic acid polymerase may be a polymerase which is capable of acting upon a DNA primer to synthesise an XNA molecule, such as a 2′OMe, MOE, PS-MOE, or LNA polymer, that is complementary to a single-stranded nucleic acid template. Such polymerases include polymerases comprising mutations corresponding to Y409G, I521L, T541G, F545L, K592A, and E664K (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations Y409G, I521L, T541G, F545L, K592A, and E664K relative to the amino acid sequence of SEQ ID NO: 1.

The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase which is capable of acting upon a DNA primer to synthesise a 2′OMe, MOE, PS-MOE, or LNA polymer, may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, Y409G, A485L, I521L, T541G, F545L, K592A, and E664K relative to the amino acid sequence of SEQ ID NO: 1.

In a particular embodiment, the nucleic acid polymerase which is capable of acting upon a DNA primer to synthesise a 2′OMe, MOE, PS-MOE, or LNA polymer, may comprise or may be of the following amino acid sequence:

MILDTDYITEDGKPVIRIFKKENGEFKIDYDRNFEPYIYALLKDDSAIEDVKKITAERHGTTVRV
VRAEKVKKKFLGRPIEVWKLYFTHPQDQPAIRDKIKEHPAVVDIYEYDIPFAKRYLIDKGLIPME
GDEELKMLAFAIATLYHEGEEFAEGPILMISYADEEGARVITWKNIDLPYVDVVSTEKEMIKRFL
KVVKEKDPDVLITYNGDNFDFAYLKKRSEKLGVKFILGREGSEPKIQRMGDRFAVEVKGRIHFDL
YPVIRRTINLPTYTLEAVYEAIFGQPKEKVYAEEIAQAWETGEGLERVARYSMEDAKVTYELGKE
FFPMEAQLSRLVGQSLWDVSRSSTGNLVEWELLRKAYERNELAPNKPDERELARRRESYAGGYVK
EPERGLWENIVYLDERSLGPSIIITHNVSPDTLNREGCEEYDVAPQVGHKFCKDFPGFIPSLLGD
LLEERQKVKKKMKATIDPIEKKLLDYRQRLIKILANSFYGYYGYAKARWYCKECAESVTAWGRQY
LETTIREIEEKFGFKVLYADGDGFLATIPGADAETVKKKAKEFLDYINAKLPGLLELEYEGFYKR
GFFVTKAKYAVIDEEDKITTRGLEIVRRDWSEIAKETQARVLEAILKHGDVEEAVRIVKEVTEKL
SKYEVPPEKLVIYKQITRDLKDYKATGPHVAVAKRLAARGIKIRPGTVISYIVLKGSGRIGDRAI
PFDEFDPAKHKYDAEYYIENQVLPAVERILRAFGYRKEDLRYQKTRQVGLGAWLKPKT (SEQ ID
NO: 20; also known as 2M polymerase).

The polymerase may be of an amino acid sequence having at least 36%, 50%, 60%, 70%, 80%, 90%, 95%, 99%, or 100% similarity or identity to the amino acid sequence of SEQ ID NO: 20, wherein residues 93, 141, 143, 409, 485, 521, 541, 545, 592, and 664 are invariant (i.e. the mutations V93Q, D141A, E143A, Y409G, A485L, I521L, T541G, F545L, K592A, and E664K, are maintained).

In a certain embodiment, there is provided a method of displaying a 2′-O-methyl ribonucleotide polymer or a 2′-O-methoxyethyl nucleotide polymer on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for 2′-O-methyl ribonucleotide or 2′-O-methoxyethyl nucleotide polymerisation, wherein
   the primer for polymerisation is a DNA primer immobilised on the substrate such a bridge is formed during polymerisation,
   the product of the polymerisation is a chain of 2′-O-methyl ribonucleotides or 2′-O-methoxyethyl nucleotides that is immobilised on the substrate via the primer, and
   the nucleic acid polymerase is a polymerase capable of acting upon a DNA primer to synthesise a 2′-O-methyl ribonucleotide polymer or a 2′-O-methoxyethyl nucleotide that is complementary to a single-stranded nucleic acid template, and comprises a sequence that has at least 80%, 90%, 95%, 99%, or 100% identity to the amino acid sequence of SEQ ID NO: 20, wherein residues 93, 141, 143, 409, 485, 521, 541, 545, 592, and 664 are invariant; and
iii) removing the first nucleic acid to result in display of the second nucleic acid on the substrate. In this embodiment, the second nucleic acid is a single-stranded 2′-O-methyl ribonucleotide polymer or a 2′-O-methoxyethyl nucleotide polymer displayed on the substrate.

The nucleic acid polymerase may be a polymerase which is capable of acting upon a DNA primer to synthesise a 2′NH₂-RNA, 2′O-methyl-RNA, 3′ deoxi-DNA (2′-5′), or 3′O-methyl-RNA polymer that is complementary to a single-stranded nucleic acid template. Such polymerases include any polymerase capable of synthesising said polymers as disclosed in Cozens et al. (Cozens, Mutschler, Nelson, Houlihan, Taylor, and Holliger, Enzymatic Synthesis of Nucleic Acids with Defined Regioisomeric 2′-5′ Linkages, Angew Chem Int Ed Engl. 2015 Dec. 14: 54 (51): 15570-15573), which is herein incorporated by reference. For instance, the polymerase may be TGLLK as disclosed in said document, or a variant thereof.

The polymerase may comprise mutations corresponding to Y409G, I521L, F545L, and E664K (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations Y409G, I521L, F545L, and E664K relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, Y409G, A485L, I521L, F545L, and E664K relative to the amino acid sequence of SEQ ID NO: 1 (the polymerase with 100% identity may be referred to as TGLLK).

The nucleic acid polymerase may be a polymerase which is capable of acting upon a DNA primer to synthesise an XNA polymer, such as a TNA polymer, that is complementary to a single-stranded nucleic acid template. Such polymerases include any polymerase capable of synthesising said polymers as disclosed in Chen and Romesberg (FEBS Lett. 2014 Jan. 21: 588 (2): 219-229) or Pinheiro et al. (Synthetic genetic polymers capable of heredity and evolution: Science. 2012 Apr. 20: 336 (6079): 341-344), each of which is herein incorporated by reference. For instance, the polymerase may be RT521 as disclosed in said documents, or a variant thereof. As disclosed in these documents, this polymerase is capable of synthesising XNA polymers other than TNA.

The polymerase may comprise mutations corresponding to E429G, I521L, and K726R (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations E429G, I521L, and K726R relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, E429G, A485L, I521L, and K726R relative to the amino acid sequence of SEQ ID NO: 1 (the polymerase with 100% identity may be referred to as RT521).

The nucleic acid polymerase may be a polymerase which is capable of acting upon a DNA primer to synthesise an XNA polymer, such as an HNA polymer, that is complementary to a single-stranded nucleic acid template. Such polymerases include any polymerase capable of synthesising said polymers as disclosed in Taylor et al. (Catalysts from synthetic genetic polymers; Nature. 2015 Feb. 19: 518 (7539): 427-430) or Pinheiro et al. (Synthetic genetic polymers capable of heredity and evolution: Science. 2012 Apr. 20: 336 (6079): 341-344), each of which is herein incorporated by reference. For instance, the polymerase may be 6G12 as disclosed in said documents, or a variant thereof. As disclosed in these documents, this polymerase is capable of synthesising XNA polymers other than HNA.

The polymerase may comprise mutations corresponding to V589A, E609K, 1610M, K659Q, E664Q, Q665P, R668K, D669Q, K671H, K674R, T676R, A681S, L704P, and E730G (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations V589A, E609K, 1610M, K659Q, E664Q, Q665P, R668K, D669Q, K671H, K674R, T676R, A681S, L704P, and E730G relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, A485L, V589A, E609K, 1610M, K659Q, E664Q, Q665P, R668K, D669Q, K671H, K674R, T676R, A681S, L704P, and E730G relative to the amino acid sequence of SEQ ID NO: 1 (the polymerase with 100% identity may be referred to as 6G12).

The nucleic acid polymerase may be a polymerase which is capable of acting upon a DNA primer to synthesise an XNA polymer, such as an HNA, AtNA, CeNA, or LNA polymer, that is complementary to a single-stranded nucleic acid template. Such polymerases include any polymerase capable of synthesising said polymers as disclosed in Taylor et al. (Catalysts from synthetic genetic polymers; Nature. 2015 Feb. 19; 518 (7539): 427-430) or Mutschler et al. (Random-sequence genetic oligomer pools display an innate potential for ligation and recombination; eLife 2018:7:e43022 DOI: 10.7554/eLife.4302), each of which is herein incorporated by reference. For instance, the polymerase may be 6G12 I521L variant (“6G12521”) as disclosed in said documents, or a variant thereof.

The polymerase may comprise mutations corresponding to I521L, V589A, E609K, I610M, K659Q, E664Q, Q665P, R668K, D669Q, K671H, K674R, T676R, A681S, L704P, and E730G (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations I521L, V589A, E609K, 1610M, K659Q, E664Q, Q665P, R668K, D669Q, K671H, K674R, T676R, A681S, L704P, and E730G relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, A485L, I521L, V589A, E609K, 1610M, K659Q, E664Q, Q665P, R668K, D669Q, K671H, K674R, T676R, A681S, L704P, and E730G relative to the amino acid sequence of SEQ ID NO: 1 (the polymerase with 100% identity may be referred to as 6G12521).

The nucleic acid polymerase may be a polymerase which is capable of acting upon a DNA primer to synthesise an XNA polymer, such as an CeNa or a LNA polymer, that is complementary to a single-stranded nucleic acid template. Such polymerases include any polymerase capable of synthesising said polymers as disclosed in Pinheiro et al. (Synthetic genetic polymers capable of heredity and evolution: Science. 2012 Apr. 20: 336 (6079): 341-344). For instance, the polymerase may be PolC7 (also known as “C7”), or a variant thereof, as disclosed in said documents.

The polymerase may comprise mutations corresponding to E654Q, E658Q, K659Q, V661A, E664Q, Q665P, D669A, K671Q, T676K, and R709K (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations E654Q, E658Q, K659Q, V661A, E664Q, Q665P, D669A, K671Q, T676K, and R709K relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, A485L, E654Q, E658Q, K659Q, V661A, E664Q, Q665P, D669A, K671Q, T676K, and R709K relative to the amino acid sequence of SEQ ID NO: 1 (the polymerase with 100% identity may be referred to as C7).

The nucleic acid polymerase may be a polymerase which is capable of acting upon a DNA primer to synthesise an XNA molecule, such as a phNA, PMO, or P-alkyl-moNA molecule, that is complementary to a single-stranded nucleic acid template. Such polymerases include any polymerase capable of synthesising a phNA molecule as disclosed in Arangundy-Franklin et al. (Nature Chemistry volume 11, pages 533-542 (2019), which is herein incorporated by reference. In particular, the polymerase may be “GV”, “GV2”, or “PGV2” (also known as “PGLVV”) as disclosed in this document, or a variant thereof.

The polymerase may comprise mutations corresponding to E429G, D455P, K487G, I521L, R606V, R613V, and K726R (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations E429G, D455P, K487G, I521L, R606V, R613V, and K726R relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, E429G, D455P, A485L, K487G, I521L, R606V, R613V, and K726R relative to the amino acid sequence of SEQ ID NO: 1 (the polymerase with 100% identity may be referred to as PGV2 or PGLVV).

The nucleic acid polymerase may be a polymerase which is capable of acting upon a DNA primer to synthesise an XNA molecule, such as a phNA, PMO, or P-alkyl-moNA molecule, that is complementary to a single-stranded nucleic acid template. In an embodiment, the polymerase may comprise mutations corresponding to N₂₆₉W, E429G, D455P, K487G, I521L, V589A, R606V, R613V, and K726R (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1).

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations N₂₆₉W, E429G, D455P, K487G, I521L, V589A, R606V, R613V, and K726R relative to the amino acid sequence of SEQ ID NO: 1. The amino acid sequence of the nucleic acid polymerase may further comprise one or more, or all, of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, N269W, E429G, D455P, A485L, K487G, I521L, V589A, R606V, R613V, and K726R relative to the amino acid sequence of SEQ ID NO: 1 (the polymerase with 100% identity may be referred to as PGLVVWA).

When using other polymerase backbones, mutations are transferred to the equivalent position as is well known in the art. For example, with reference to the exemplary polymerase 6G12, the following table illustrates how the transfer of mutations to alternate backbones may be carried out. The table shows Pol6G12 mutations and structural equivalent positions in other PolBs. The mutations found in Pol6G12 are shown against the underlying sequence of the wild-type Tgo. The structurally equivalent residue in other well-studied B-family polymerases is given. Residues that were not mapped to equivalent positions are shown as N.D.

| | | | |
|---|---|---|---|
| Tgo (1TGO) | Pol6G12 | RB69 (1IG9) | E. coli (3MAQ) |

| | | | | |
|---|---|---|---|---|
| V | 589 | A | 703 | 604 |
| E | 609 | K | 732 | N.D. |
| I | 610 | M | 733 | N.D. |
| K | 659 | Q | 778 | 681 |
| E | 664 | Q | 783 | 686 |
| Q | 665 | P | 784 | 687 |
| R | 668 | K | 788 | 690 |
| D | 669 | Q | 789 | 691 |
| K | 671 | H | N.D. | 693 |
| K | 674 | R | 792 | N.D. |
| T | 676 | R | 801 | 700 |
| A | 681 | S | 806 | 705 |
| L | 704 | P | 835 | 733 |
| E | 730 | G | 869 | 750 |

Mutating may refer to the substitution or truncation or deletion of the residue, motif or domain referred to. In a particular embodiment, the mutation is a substitution of one type of amino acid residue for another type of amino acid residue.

The polymerase may be a fragment of a polymerase which retains the polymerase function.

The conditions suitable for polymerisation of step ii) a) may be cycles involving a denaturation step, an annealing step, and an amplification step. The denaturation step may be the application of a denaturation buffer, for instance a buffer containing 98% formamide and/or NaOH. The NaOH may be at a concentration of greater than or equal to 1 mM NaOH, preferably 10 mM NaOH. The denaturation buffer may also comprise EDTA, for instance 1 mM EDTA. The annealing step may be the application of a premix buffer, which may include the same components as the amplification buffer without the NTPs or the polymerase. For instance, the premix buffer may include 2 M Betaine, 20 mM Tris, 10 mM Ammonium sulfate, 6 mM MgSO₄, 0.1% Triton-X, 1.3% DMSO, and 18.1 U/ml RNAse inhibitor, at pH 8.8. The amplification step may involve contacting the substrate-bound nucleic acids with the polymerase, RNA nucleotide triphosphates, and a suitable amplification buffer. As an example, the amplification buffer may include 2M Betaine, 20 mM Tris, 10 mM Ammonium sulfate, 6 mM MgSO₄, 0.1% Triton-X, 1.3% DMSO, 625 uM NTPs, 10 nM TGK polymerase, and 18.1 U/ml RNAse inhibitor, at pH 8.8. In another example, the amplification buffer may include 20 mM Tris-HCl, 10 mM (NH₄)₂SO₄, 10 mM KCl, 2 mM MgSO₄, 0.1% Triton X-100, 200 uM faNTPs, 10 nM D4YK pol, pH 8.8. In a third example, the amplification buffer may include 200 uM 2′OMe NTPs, 10 nM 2M polymerase, 2 M Betaine, 20 mM Tris, 10 mM Ammonium sulfate, 6 mM MgSO4, 0.1% Triton-X, 1.3% DMSO, pH 8.8. In some embodiments, at least 5, 10, 12, 15, 20, or 25 cycles of bridged polymerisation are carried out. In some embodiments, the RNAse inhibitor may be or may comprise SuperaseIn, RNAseOUT, RNasein, RiboSafe or any other commercially available product that does not inhibit the polymerase activity.

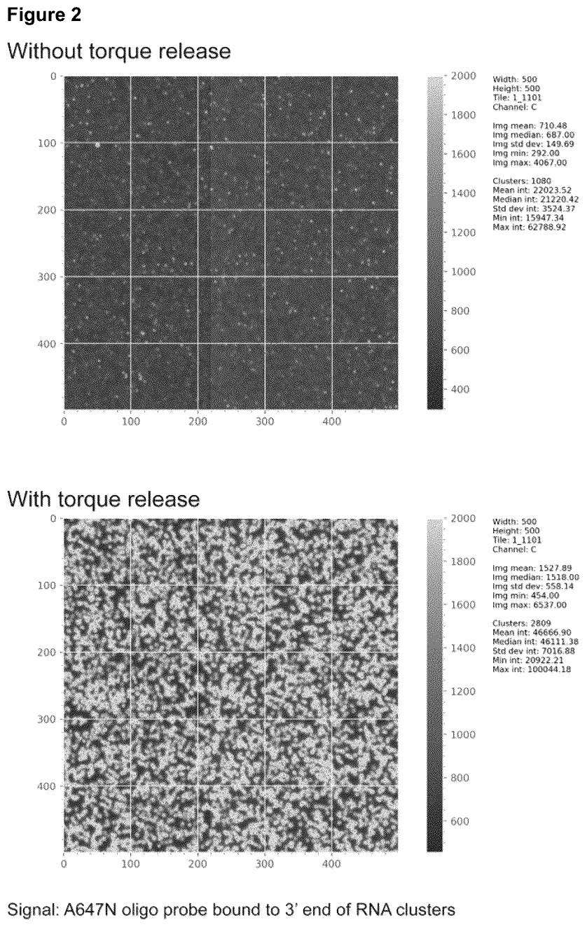

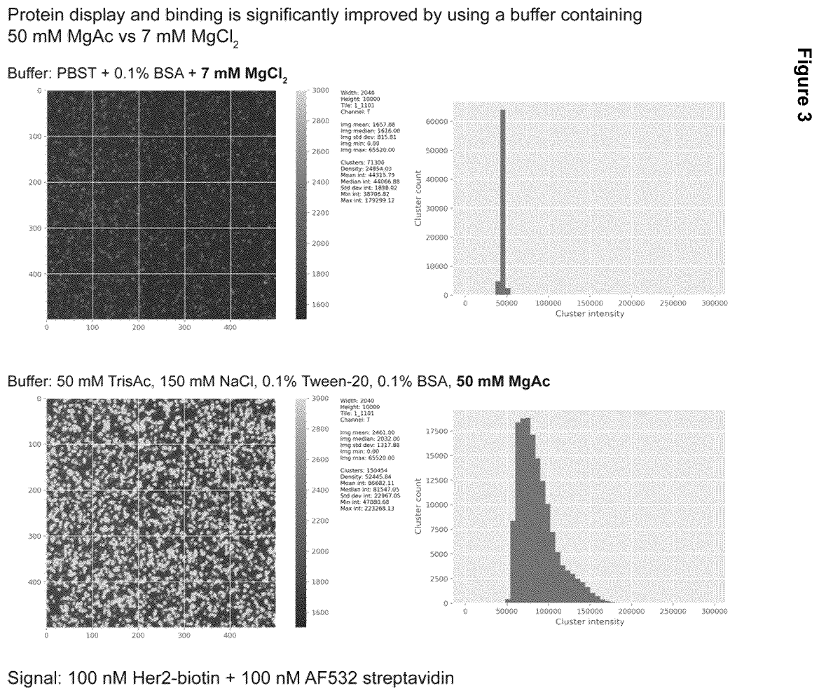

In addition to the preceding disclosure, the inventors provide further steps which improve the synthesis of the non-DNA polymers in the methods of the invention. These further steps are particularly relevant to long constructs. Without being bound to theory, the inventors suspect that during non-DNA or RNA synthesis in a bridge, the dsDNA:RNA complex (or other non-DNA nucleic acid complex) starts to build up a significant amount of torque that slows down and eventually stalls the polymerase. The inventors have overcome this issue. For instance, see FIG. 2, which shows the improvement associated with this aspect of the invention.

Thus, in an embodiment, the method further comprises a step, which takes place after the initial polymerisation step or cycles, wherein the first nucleic acid is cleaved. The cleavage enables the bridge to be linearized, releasing the torque, while retaining the first nucleic acid. In embodiments wherein a polypeptide is displayed, the cleavage site should be after the open reading frame encoding the polypeptide to avoid interference with the further rounds of polymerisation. As such, the cleavage site within the first nucleic acid is positioned 5′ to the sequence within the first nucleic acid corresponding to the encoded polypeptide. In particular embodiments, the cleavage site is within the immobilised adapter/primer that links the first nucleic acid to the substrate.

In some embodiments, this step is applied to methods involving a first nucleic acid that is greater than 500, 600, 700, 800, 900, 1000, 1100, 1200, 1300, 1400, or 1500 nucleotides in length. In a particular embodiment, this step is applied to methods involving a first nucleic acid that is greater than 800 or 900 nucleotides in length. This is particularly relevant to embodiments where the second nucleic acid is an RNA molecule, because the RNA molecules may encode a polypeptide and thus are commonly longer.

The cleavage may be any which allows targeted cleavage of the first nucleic acid in a manner that does not alter other components, such as the newly formed nucleic acid strand. In particular embodiments, the cleavage site may be incorporated into the adapter/primer that links the first nucleic acid to the substrate. For instance, the cleavage site may be 2-deoxyuridine which can be cut with the Uracil-Specific Excision Reagent (USER) enzyme. Alternatively, the cleavage site may be 8-oxoguanine which can be cut with formamidopyrimidine DNA glycosylase (Fpg). When Fpg is used to cleave the first nucleic acid, the inventors have found that cleavage efficiency is increased when the cleavage site is double-stranded DNA. As such, in a particular embodiment, a third nucleic acid is hybridised to the cleavage site. The third nucleic acid may be a DNA oligo which is complementary to the sequence spanning the cleavage site, for instance a DNA oligo which can hybridise to the 8-oxoguanine site in the Illumina P7 adapter. The 3′ end of the third nucleic acid may be modified to prevent extension of third nucleic acid during the method. For instance, the 3′ end of the third nucleic acid may be phosphory lated.

After cleavage, the bridge is present in a linearized state. The temperature may be raised to denature the bridge.

The linearized product is then re-contacted with a polymerase under conditions suitable for polymerisation. The inventors found that these steps increase the efficiency of polymerisation and led to a higher yield of complete non-DNA molecules.

In a particular embodiment, the first nucleic acid is contacted with a nucleic acid polymerase under conditions suitable for polymerisation, wherein the primer for polymerisation is immobilised on the substrate such a bridge is formed during polymerisation, and wherein at least 5, 10, 12, 15, 20, or 25 cycles of bridged polymerisation are carried out. In the embodiments of the Examples, 12 cycles are carried out, but this number may be increased. After the bridged amplification cycles, the first nucleic acid is cleaved and the bridge is linearized. Polymerisation is then carried out again. Polymerisation after the linearization step need not comprise a denaturation step, and the lack of the denaturation step avoids the disassociation of, for instance, the DNA: RNA duplex.

The steps of cleavage, linearization, and continued polymerisation may be cycled. For instance, two cycles may be carried out. In other embodiments, 3, 4, 5 or more cycles are carried out.

As such, in a particular embodiment, there is provided a method of displaying an RNA molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for RNA polymerisation, wherein
   the primer for polymerisation is a DNA primer immobilised on the substrate such a bridge is formed during polymerisation,
   the product of the polymerisation is a chain of RNA nucleotides that is immobilised on the substrate via the primer,
   the nucleic acid polymerase is any polymerase capable of acting upon a DNA primer to synthesise an RNA molecule that is complementary to a single-stranded nucleic acid template, including any disclosed herein, and
   at least 5, 10, 12, 15, 20, or 25 cycles of bridged polymerisation are carried out;
   b) cleaving the first nucleic acid and linearizing the bridge; and
   c) contacting the linearized product of step b) with a polymerase under conditions suitable for RNA polymerisation;
   d) carrying out step b) followed by step c) for at least one further cycle; and
iii) removing the first nucleic acid to result in display of the second nucleic acid on the substrate. The first nucleic acid may comprise an antisense sequence encoding a polypeptide. The cleavage of step ii) b) may be at a site that is 5′ to the encoded polypeptide sequence.

The surprisingly effective steps for polymerisation using a substrate-bound template are applicable where the polymerase used is not capable of acting upon a DNA primer. For instance, the steps of polymerisation, linearization, followed by additional polymerisation are also applicable to other methods that make use of polymerases that act upon, for instance, RNA or non-DNA primers.

As such, in an aspect of the invention, there is provided a method of displaying non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
   a) contacting the first nucleic acid with a nucleic acid polymerase under conditions suitable for polymerisation, wherein
   the primer for polymerisation is immobilised on the substrate such a bridge is formed during polymerisation, and
   the product of the polymerisation is a chain of nucleotides that is immobilised on the substrate via the primer;
   b) cleaving the first nucleic acid and linearizing the bridge; and
   c) contacting the linearized product of step b) with a polymerase under conditions suitable for polymerisation; and
iii) removing the first nucleic acid to result in display of the second nucleic acid on the substrate. For embodiments where the second nucleic acid molecule is RNA, the first nucleic acid may comprise an antisense sequence encoding a polypeptide. The cleavage of step ii) b) may be at a site that is 5′ to the encoded polypeptide sequence.

Further details of the above described method may be any as disclosed herein. For instance, the number of cycles of bridge amplification and/or cycles of linearization and re-polymerisation may the as discussed in the preceding passages. The buffers may be as disclosed in the preceding passages. However, as mentioned, while the polymerase may be any of those disclosed herein, this method is not limited and the polymerase may be, for instance, 3D^{pol} polymerase.

In step iii) of the methods of displaying a non-DNA nucleic acid molecule on a substrate, the first nucleic acid is removed such that the newly synthesised nucleic acid molecule is present as a single-stranded nucleic acid molecule displayed on the substrate. Various techniques are available for removing the first nucleic acid and, in a particular embodiment, the first nucleic acid is removed by the use of a denaturation reagent. For instance, the denaturation reagent may be a buffer comprising 1-500 mM, 10-400 mM, 25-300 mM, 50-200 mM, or 75-125 mM NaOH. In an embodiment, the denaturation reagent comprises 100 mM NaOH. The denaturation reagent may comprise 0-20 mM EDTA. In an embodiment, the denaturation reagent comprises 5 mM EDTA. The denaturation reagent may comprise 100 mM NaOH and 5 mM EDTA and the substrate-nucleic acid complex may be contacted with said buffer. In particular embodiments, step iii) does not comprise the use of DNasel.

The methods of displaying a non-DNA nucleic acid molecule thus result in a substrate with an immobilised nucleic acid molecule on the surface. As discussed herein, the nucleic acid molecules may be present in clusters and sequencing and position information may have been obtained. The displayed nucleic acid molecules may form a library. For instance, a library of aptamers, such as RNA, XNA, FANA, ANA, or 2′-OMe aptamers. The library may be of XNAzymes, for instance XNAzymes comprising enzymes made of FANA polymers or any other XNA polymer. Alternatively, the nucleic acid molecules themselves may be displayed for analysis. For instance, the binding of a molecule to the non-DNA nucleic acid molecules may be assessed.

In embodiments that involve display of XNAzymes, nucleic acid oligos, e.g. DNA oligos, may be annealed to any 5′ and 3′ adaptors to ensure the XNAzyme is not interfered with by the adaptors.

Some embodiments result in an RNA molecule being displayed on a substrate, and the RNA molecule may encode a polypeptide. As discussed herein, the RNA molecules may be present in clusters and sequencing and position information may have been obtained. The RNA clusters may form a library of encoded polypeptides. In some embodiments, the RNA molecule encodes a peptide or protein of between 1 and 25 kDa in size. In other embodiments, a library of peptides or proteins of between 1 and 25 kDa in size is displayed. As examples, the library may be of scFVs, peptides, fibronectin type III domains (FN3 domains), or single-domain antibodies (sdAbs, also known as nanobodies). Other scaffolds that can be displayed include affibodies, darpins, fynomers, OBodies, and avimers.

To obtain said libraries, the methods of displaying an RNA molecule on a substrate may start with a substrate wherein the immobilised first nucleic acid is a plurality of first nucleic acids encoding a plurality of polypeptides. The first nucleic acids may be present in clusters which have been, at least in part, sequenced.

Once the immobilised single-stranded RNA molecule has been obtained, a probe may optionally be annealed to the single-stranded RNA molecule. For instance, a nucleic acid probe which is complementary to the 3′ end of the second nucleic acid may be hybridised to the second nucleic acid. The hybridisation site should preferably not be within the open reading frame of the encoded polypeptide. The hybridisation site may be positioned away from the stop codon of the open reading frame to avoid steric clashes between the probe and the ribosome. For instance, the hybridisation site may be at least 10, 15, 20, 25, 30, 35, or 40 nucleotides from the stop codon. In a particular embodiment, the hybridisation site is at least 30 nucleotides from the stop codon. The probe may be labelled, for instance fluorescently labelled, such that RNA synthesis may be verified, visualised, and quantified.

In further embodiments, the inventors make use of such polymerases to generate clusters of RNA molecules that are immobilised to a substrate, such as a flow cell, and go on to show surprisingly effective display of polypeptides translated from said RNA clusters.

Thus, the methods may further comprise the step of contacting the second nucleic acid, which is the newly formed RNA molecule, with a ribosome under conditions suitable for translation of an encoded polypeptide. This allows in vitro translation of the RNA sequence to form the polypeptide itself.

The displayed polypeptide may comprise or consist of canonical amino acids. The displayed polypeptide may comprise non-canonical amino acids. The displayed polypeptide may comprise unnatural amino acids. In an embodiment, the displayed polypeptide comprises any combination of canonical amino acids, non-canonical amino acids, and/or unnatural amino acids.

The second nucleic acid may comprise a ribosome binding site 5′ to an open reading frame. For instance, the second nucleic acid may comprise a Shine-Dalgarno sequence.

There have been attempts in the prior art to provide methods for displaying large numbers of polypeptides on surfaces in a manner that is suitable for high-throughput screening and analysis. However, these methods suffer from drawbacks and in particular suffer from inefficient translation of the polypeptides or instability of the displayed polypeptides. The inventors provide herein further techniques for the translation of RNA molecules displayed on substrate, and overcome the deficiencies of the prior art.

The inventors have identified that in vitro translation and folding of certain polypeptides may be inefficient. This is particularly relevant to larger folded polypeptides, such as scFvs. In order to improve said translation and folding, the inventors have identified that trimethylamine N-oxide (TMAO) may be included in the in vitro translation buffer. In particular, the inventors identified that a TMAO concentration of 0.05 M to 1.5 M enhanced the yield when performing in vitro translation at 37° C. In addition, the in vitro translation should take place in a buffer which has minimal or no RNAse activity.

Thus, in an embodiment, the method comprises contacting the second nucleic acid with a ribosome under conditions suitable for translation of the encoded polypeptide, wherein the conditions comprise trimethylamine N-oxide (TMAO). The TMAO may be at a concentration of 0.05 M to 1.5 M or 0.05 M to 1.2 M. The TMAO concentration may be 0.05 M to 1.5 M, 0.1 M to 1.2 M, 0.15 M to 1 M, 0.2 M to 0.8 M, 0.25 M to 0.6 M, 0.3 M to 0.5 M, or 0.35 M to 0.45 M. In an embodiment, the TMAO concentration is about 0.4 M.

As an alternative, the inventors have found that dimethylsulfoxide (DMSO) may improve in vitro translation when present in the translation buffer. For instance, 10% DMSO may be included in the translation buffer. The inventors found an improvement when including DMSO during translation of scFvs but did not find an improvement for all types of encoded proteins.

The surprisingly effective steps for translation of an immobilised RNA molecule are also applicable to other methods. As such, in an aspect of the invention, there is provided a method of displaying a polypeptide on a substrate, comprising:
i) providing a first nucleic acid comprising an antisense sequence encoding a polypeptide, wherein the first nucleic acid is immobilised on a substrate, and wherein the first nucleic acid is oriented such that the 5′ end is proximal and the 3′ end is distal to the point of immobilisation:
ii) generating a second nucleic acid that is complementary to the first nucleic acid, wherein the generation of the second nucleic acid comprises:
   contacting the first nucleic acid with a nucleic acid polymerase under conditions suitable for RNA polymerisation, wherein
   the primer for polymerisation is immobilised on the substrate such a bridge is formed during polymerisation, and
   the product of the polymerisation is a chain of RNA nucleotides that is immobilised on the substrate via the primer:
iii) removing the first nucleic acid to result in display of the second nucleic acid on the substrate; and
iv) contacting the second nucleic acid with a ribosome under conditions suitable for translation of the encoded polypeptide, wherein the conditions of step iv) comprise TMAO. Optionally the TMAO is at a concentration of 0.05 M to 1.5 M or 0.05 M to 1.2 M. In an embodiment, the TMAO concentration is 0.4 M.

Further details of the above described method may be any as disclosed herein. As an alternative, the TMAO may be replaced with DMSO, for instance 10% DMSO.

The encoded polypeptide may be present as an open reading frame ending in a stop codon. Translation will stall at the stop codon and the ribosome may then be stabilised. The ribosome may be stabilised by contacting the complex with a stabilisation buffer, such as a buffer comprising Mg at a concentration equivalent to at least or greater than 7 mM MgCl₂.

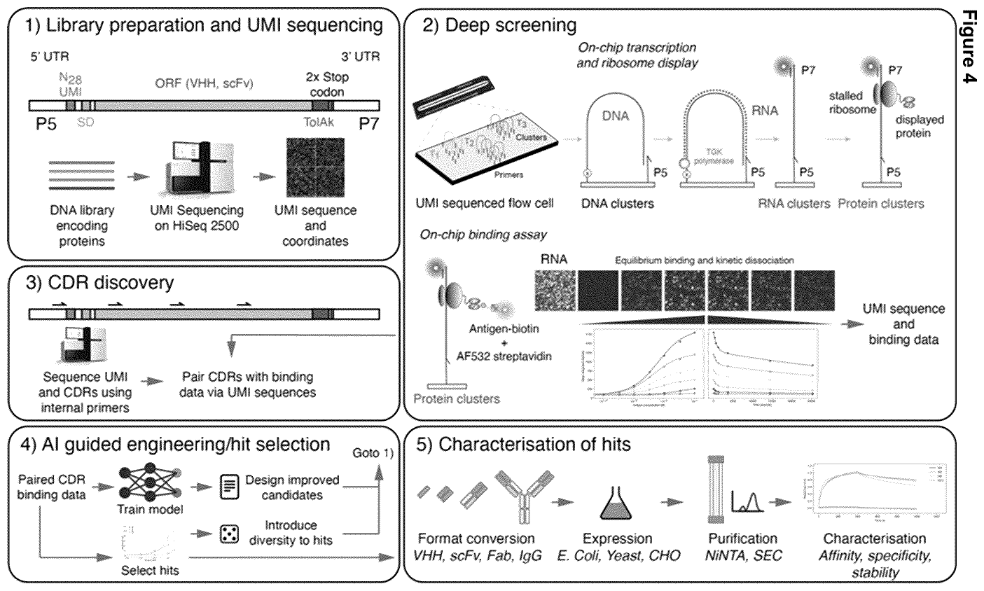

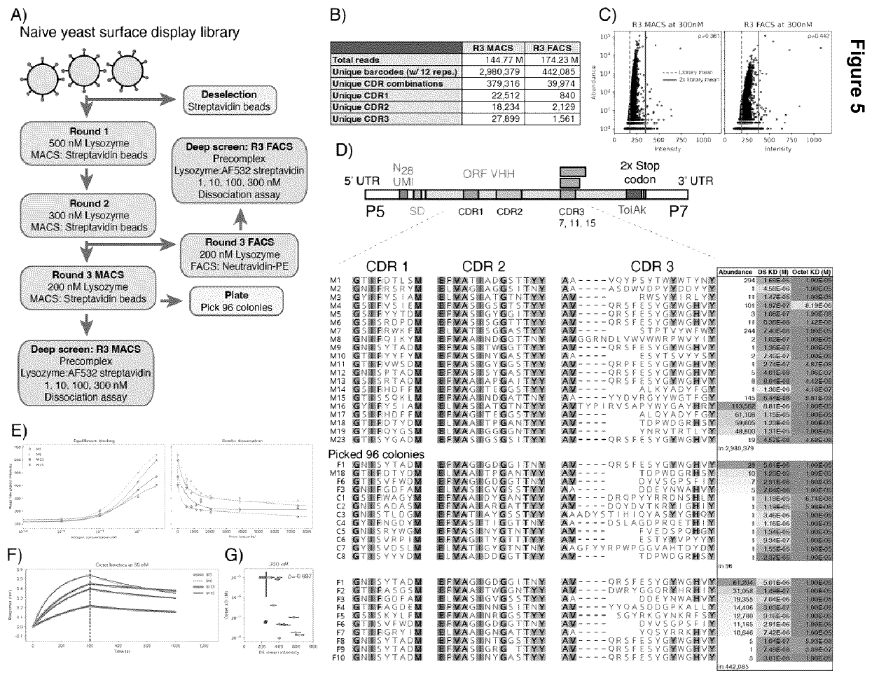

Ribosome stabilisation buffers comprising more than 7 mM MgCl₂ are unsuitable for use with prior art methods which rely on DNA-RNAP-RNA complexes that cannot be denatured. However, the present inventors have found that higher Mg concentrations are associated with increased display and stabilisation efficiency and are suitable for use in the present methods (see, for instance, FIG. 3). For instance, the present inventors observed a 30-fold increase in ribosome display efficiency in the systems of the invention when comparing 7 mM MgCl2 with 50 mM MgAc. Thus, the stabilisation buffer may comprise 8, 9, 10, 15, 20, 25, 30, 40, 50, 60, 70, 80, 90, or 100 mM MgCl₂ or MgAc. In some embodiments, the buffer has a magnesium concentration which is equivalent to or greater than 8, 9, 10, 15, 20, 25, 30, 40, 50, 60, 70, 80, 90, or 100 mM MgCl₂ or MgAc. The buffer may have a magnesium concentration that is greater than that provided by 7 mM MgCl₂. In some embodiments, the buffer has a magnesium concentration which is or is equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgCl₂ or MgAc.

The ribosome stabilisation buffer may be phosphate buffered saline comprising the aforementioned magnesium concentrations. The buffer may further comprise Tween 20 or Triton X-100.

In a particular embodiment, the ribosome display buffer may contain 50 mM TrisAc (Tris (hydroxymethyl)aminomethane acetate), 150 mM NaCl, 0.1% Tween 20, 0.1% BSA, 20 U/ml RNase inhibitor, a magnesium concentration disclosed herein, and be pH 7.5. The magnesium concentration may be provided by 50 mM MgAc (Magnesium acetate).

Such methods result in a polypeptide being displayed on the surface of the substrate. As discussed herein, a library of polypeptides, such as a library of scFv molecules may be displayed on the surface.

The polypeptide displayed may be 5 to 25 kDa, 10 to 25 kDa, 15 to 25 kDa, or 20 to 25 kDa. In some embodiments, the displayed polypeptide is not larger than 25 kDa. In particular embodiments, the polypeptide may be larger than 15 kDa.

The substrate surface with the polypeptide displayed may be washed and blocked. Suitable blocking agents include bovine serum albumin, casein, recombinant bovine serum albumin, and the like.

The substrate surface displaying the polypeptide may be used for further studies. For instance, if the surface displays a library of target-binding proteins, or potential target-binding proteins, a candidate target, antigen, peptide, or protein may be contacted to the surface to determine the binding characteristics of the displayed target-binding fragments. The candidate may be fluorescently labelled or detectable in another manner. Thus, the displayed the library may be used to analyse binding properties.

The invention is also not limited to the measurement of binding properties, and the invention may be used to analyse any other property. For instance, a library encoding variants of an enzyme may be prepared, and the library may be used to analyse enzymatic activity.

In a particular embodiment, there is provided a method of displaying a polypeptide on a substrate, comprising:
i) providing a first nucleic acid comprising an antisense sequence encoding a polypeptide, such as an scFv, wherein the first nucleic acid is immobilised on a substrate, and wherein the first nucleic acid is oriented such that the 5′ end is proximal and the 3′ end is distal to the point of immobilisation:
ii) generating a second nucleic acid that is complementary to the first nucleic acid, wherein the generation of the second nucleic acid comprises:
   a) contacting the first nucleic acid with a nucleic acid polymerase under conditions suitable for RNA polymerisation, wherein
   the primer for polymerisation is a DNA primer immobilised on the substrate such a bridge is formed during polymerisation,
   the product of the polymerisation is a chain of RNA nucleotides that is immobilised on the substrate via the primer, and
   the nucleic acid polymerase is a polymerase capable of acting upon a DNA primer to synthesise an RNA molecule that is complementary to a single-stranded nucleic acid template, for instance TGK;
   b) cleaving the first nucleic acid at a site that is 5′ to the encoded polypeptide sequence and linearizing the bridge; and
   c) contacting the linearized product of step b) with a polymerase under conditions suitable for RNA polymerisation:
iii) removing the first nucleic acid to result in display of the second nucleic acid on the substrate:
iv) contacting the second nucleic acid with a ribosome under conditions suitable for translation of the encoded polypeptide, wherein the conditions of step iv) comprise TMAO optionally at a concentration between 0.05 and 1.5 M; and
v) stabilising the ribosome-polypeptide complex with a ribosome display buffer, optionally comprising a magnesium concentration greater than 7 mM MgCl₂. In an embodiment, the TMAO concentration is 0.05 M to 1.2M. In a particular embodiment, the TMAO concentration is 0.4 M.

As discussed herein, the methods of displaying a biomolecule on a substrate involve the provision of a first nucleic acid which is immobilised onto a substrate. The first nucleic acid may be present as part of a clonal cluster and at least some sequencing and position information may have been obtained. Methods for obtaining nucleic acids immobilised in this manner, and for obtaining the aforementioned information, are known in the art. However, the inventors provide herein particularly improved methods that are optimised for the downstream methods disclosed herein. In particular, it is desirable to be able to produce longer immobilised nucleic acid sequences, for instance of a length of 1.2 Kbp or more. The inventors provide methods that are improved for the production of said long constructs.

As such, in an embodiment, the first nucleic acid immobilised on the substrate as provided in step i) is generated by:
1) providing a template nucleic acid encoding a polypeptide sequence:
2) hybridising the template nucleic acid to a primer immobilised to a substrate;
3) contacting the hybridised template nucleic acid with a polymerase under conditions suitable for the extension of the immobilised primer to synthesise the first nucleic acid which is a chain of nucleotides that are complementary to the template;
4) performing bridge amplification of the first nucleic acid to generate clusters of the first nucleic acid; and
5) sequencing at least a part of the first nucleic acid.

The template nucleic acid may have an adapter oligonucleotide at the 5′ end and at the 3′ end. For instance, if the substrate is an Illumina flow cell, the adapters may be the P5 and P7 adapters. The primers immobilised to the substrate may be complementary to at least a part of the template nucleic acid, such as an adapter.

The bound template nucleic acid is then contacted with a polymerase under conditions suitable for the extension of the immobilised primer to synthesise the first nucleic acid which is a chain of nucleotides that are complementary to the template. As such, the first nucleic acid is an extension of the immobilised primer. The first nucleic acid and template nucleic acid may then be denatured to result in a single-stranded first nucleic acid immobilised to the substrate.

Bridge amplification may then be used to generate clonal clusters of the first nucleic acid. Bridge amplification may comprise cycles of an annealing step, an amplification step, and a denaturation step. As an example, the amplification may include the following features: 28-35 cycles, an extension time of 1-120 seconds, an amplification buffer comprising Mg at a concentration equivalent to 2-6 mM MgSO₄, and/or a denaturation buffer comprising 95-99.9% Formamide with or without the addition of 1-10 mM NaOH and 1-5 mM EDTA.

The inventors have discovered that the following features may be used to particularly optimise this step for the downstream RNA/polypeptide display features: 32-35 cycles, an extension time of 60-120 seconds, an amplification buffer comprising Mg at a concentration equivalent to 2-6 mM MgSO₄, and a denaturation buffer comprising 95-99.9% Formamide with or without the addition of 1-10 mM NaOH and 1-5 mM EDTA.

As such, in an embodiment, the first nucleic acid immobilised on the substrate as provided in step i) is generated by:
1) providing a template nucleic acid encoding a polypeptide sequence:
2) hybridising the template nucleic acid to a primer immobilised to a substrate:
3) contacting the hybridised template nucleic acid with a polymerase under conditions suitable for the extension of the immobilised primer to synthesise the first nucleic acid which is a chain of nucleotides that are complementary to the template:
4) performing bridge amplification of the first nucleic acid to generate clusters of the first nucleic acid, wherein the bridge amplification is carried out for 32-35 amplification cycles, has an extension time of 60-120 seconds per cycle, comprises the use of an amplification buffer comprising Mg at a concentration equivalent to 2-6 mM of MgSO₄, and comprises the use of a denaturation buffer comprising 95-99.9% Formamide, optionally 1-10 mM NaOH, and optionally 1-5 mM EDTA; and
5) sequencing at least a part of the first nucleic acid.

In a particular embodiment, the bridge amplification comprises 32 cycles. The extension time may be 60 seconds. The amplification buffer may comprise Mg at a concentration equivalent to 6 mM MgSO₄. The denaturation buffer may comprise 98% Formamide, 10 mM NaOH, and 1 mM EDTA.

The amplification buffer may be: 2 M Betaine, 20 mM Tris, 10 mM Ammonium sulfate, 6 mM MgSO₄, 0.1% Triton-X, 1.3% DMSO, 200 uM dNTPs, 80 U/ml Bst 2.0, pH 8.8.

The polymerase may be the Bst large fragment, Bst 2.0 polymerase or Bst 3.0 polymerase (New England Biolabs).

After cluster generation, the double-stranded bridges may be linearized and denatured according to techniques known in the art. At least a part of the first nucleic acid may then be sequenced in a standard manner. For instance, the first nucleic acid may comprise a primer binding site followed by a unique molecular indicator or barcode sequence, and the barcode sequence may be sequenced. The barcode sequence may be a 15-30 nucleotide random barcode.

After sequencing, the sequencing product may be removed. The 3′ phosphate of the immobilised phosphate may be deprotected to allow for the further methods of the invention to be applied. For instance, if an Illumina flow cell and reagents are used, the 3′ phosphate of the P5 primer may be deprotected. The enzyme T4 PNK may be used for deprotection.

As described, the inventors provide an optimised method of generating clusters of nucleic acid molecules immobilised on a substrate which is particularly useful for certain downstream applications. As such, in an aspect of the invention, there is provided a method of preparing clusters of substrate-bound nucleic acids, comprising:
1) providing a template nucleic acid encoding a polypeptide sequence:
2) hybridising the template nucleic acid to a primer immobilised to a substrate:
3) contacting the hybridised template nucleic acid with a polymerase under conditions suitable for the extension of the immobilised primer to synthesise the first nucleic acid which is a chain of nucleotides that are complementary to the template; and
4) performing bridge amplification of the first nucleic acid to generate clusters of the first nucleic acid, wherein the bridge amplification is carried out for 32-35 amplification cycles, has an extension time of 60-120 seconds per cycle, comprises the use of an amplification buffer comprising Mg at a concentration equivalent to 2-6 mM of MgSO₄, and comprises the use of a denaturation buffer comprising 95-99.9% Formamide and optionally 1-10 mM NaOH, and optionally 1-5 mM EDTA.

In a particular embodiment, the bridge amplification comprises 32 cycles. The extension time may be 60 seconds. The amplification buffer may comprise Mg at a concentration equivalent to 6 mM MgSO₄. The denaturation buffer may comprise 98% Formamide, 10 mM NaOH, and 1 mM EDTA.

The methods of preparing clusters of substrate-bound nucleic acids disclosed herein may be used to display nucleic acids of at least 0.5, 1, 1.2 or 1.5 Kbp in length. The methods may be used to display nucleic acids of 1 to 1.5 Kbp, 1.1 to 1.3 Kbp, or 1.2 Kbp in length.

In an aspect of the invention, there is provided a substrate displaying a non-DNA nucleic acid molecule, such as an XNA, an FANA, a 2′OMe, or an RNA molecule, which is obtained or obtainable by any of the methods disclosed herein.

In an aspect of the invention, there is provided a substrate displaying a polypeptide which is obtained or obtainable by any of the methods disclosed herein.

In another aspect of the invention, there is provided the use of a nucleic acid polymerase to extend a DNA primer immobilised on a substrate to synthesise a non-DNA nucleic acid molecule that is complementary to a single-stranded nucleic acid template.

Features disclosed in connection with the methods of the invention may also be applied to this aspect of the invention. For instance, any of the features relevant to an RNA or an XNA polymerase.

For example, the nucleic acid polymerase may comprise an amino acid sequence having at least 36%, 50%, 60%, 70%, 80%, 90%, 95%, 99%, or 100% similarity or identity to the amino acid sequence of SEQ ID NO: 1, further comprising mutations allowing the polymerisation of at least one type of XNA nucleotide or RNA nucleotide. The nucleic acid polymerase may comprise one or more, or all, of the following mutations: V93Q, D141A, E143A, and A485L.

In particular, the polymerase may be of an amino acid sequence having at least 36%, 50%, 60%, 70%, 80%, 90%, 95%, 99%, or 100% similarity or identity to the amino acid sequence of SEQ ID NO: 3, wherein residues 93, 141, 143, 409, 485, and 664 are invariant (i.e. the mutations V93Q, D141A, E143A, Y409G, A485L, and E664K are maintained). The polymerase may be of an amino acid sequence having at least 36%, 50%, 60%, 70%, 80%, 90%, 95%, 99%, or 100% similarity or identity to the amino acid sequence of SEQ ID NO: 4, wherein residues 93, 141, 143, 403, 485, 657, 658, 659, 663, 664, 669, 671, and 676 are invariant (i.e. the mutations V93Q, D141A, E143A, L403P, A485L, P657T, E658Q, K659H, Y663H, E664K, D669A, K67IN, and T676I, are maintained). The polymerase may be of an amino acid sequence having at least 36%, 50%, 60%, 70%, 80%, 90%, 95%, 99%, or 100% similarity or identity to the amino acid sequence of SEQ ID NO: 20, wherein residues 93, 141, 143, 409, 485, 521, 541, 545, 592, and 664 are invariant (i.e. the mutations V93Q, D141A, E143A, Y409G, A485L, I521L, T541G, F545L, K592A, and E664K, are maintained). In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, Y409G, A485L, I521L, F545L, and E664K relative to the amino acid sequence of SEQ ID NO: 1. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, E429G, A485L, I521L, and K726R relative to the amino acid sequence of SEQ ID NO: 1. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, A485L, V589A, E609K, 1610M, K659Q, E664Q, Q665P. R668K, D669Q, K671H, K674R. T676R, A681S, L704P, and E730G relative to the amino acid sequence of SEQ ID NO: 1. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, A485L, I521L, V589A, E609K, 1610M, K659Q, E664Q, Q665P. R668K, D669Q, K671H, K674R, T676R, A681S, L704P, and E730G relative to the amino acid sequence of SEQ ID NO: 1. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A. A485L, E654Q, E658Q, K659Q, V661A, E664Q, Q665P. D669A, K671Q. T676K, and R709K relative to the amino acid sequence of SEQ ID NO: 1. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, E429G, D455P, A485L, K487G, I521L, R606V, R613V. and K726R relative to the amino acid sequence of SEQ ID NO: 1. The polymerase may be Bst. The polymerase may be PGLVVWA. In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, N₂₆₉W, E429G. D455P. A485L, K487G, I521L, V589A, R606V, R613V, and K726R relative to the amino acid sequence of SEQ ID NO: 1.

In a further aspect of the invention, there is provided a nucleic acid polymerase comprising mutations corresponding to N₂₆₉W, E429G, D455P, K487G, I521L, V589A, R606V, R613V, and K726R (described relative to SEQ ID NO: 1) in the backbone of any polymerase from the polB family. In particular embodiments, the backbone is any polB polymerase excluding viral polymerases. The backbone may be of a polymerase from the Archaeal Thermococcus and/or Pyrococcus genera. The polymerase may be a variant of the polymerase from T. gorgonarius (Tgo) (SEQ ID NO: 1). The polymerase of this aspect of the invention may be associated with efficient polymerisation of XNA molecules, such as phNA, PMO, or P-alkyl-moNA polymers. The polymerase of this aspect of the invention may be capable of synthesising said polymers as strands that are complementary to a nucleic acid template, such as a DNA template.

The polymerase may be of an amino acid sequence having at least 36%, 50%, 60%, 70%, 80%, 90%, 95%, 99%, or 100% similarity or identity to the amino acid sequence of SEQ ID NO: 1, wherein said amino acid sequence comprises the mutations N₂₆₉W, E429G, D455P, K487G, I521L, V589A, R606V, R613V, and K726R relative to the amino acid sequence of SEQ ID NO: 1. The amino acid sequence of the nucleic acid polymerase may further comprise one or more of the following mutations: V93Q, D141A, E143A, and A485L. These mutations are discussed herein elsewhere.

In a particular embodiment, the nucleic acid polymerase may be of an amino acid sequence having at least 80%, 90%, 95%, 99%, or 100% similarity or identity to the amino acid sequence of SEQ ID NO: 1, wherein said amino acid sequence comprises the mutations V93Q, D141A, E143A, N₂₆₉W, E429G, D455P, A485L, K487G, I521L, V589A, R606V, R613V, and K726R relative to the amino acid sequence of SEQ ID NO: 1.

In an aspect of the invention, there is provided a method of screening a substrate displaying a plurality of biomolecules, wherein the substrate is any as disclosed herein or obtainable by any method disclosed herein, and wherein the biomolecules form a library. The library may be any as disclosed herein. For instance, the library may comprise a plurality of variants of a parental nucleic acid or polypeptide sequence.

The screening disclosed herein may comprise measuring the affinity for a ligand or a target molecule, or measuring an enzymatic function, of the displayed biomolecules. For instance, the screening may comprise measuring the affinity of displayed variants of a parental scFv, or other binding polypeptide, for a target ligand. Alternatively, the screening may comprise measuring an enzymatic function, such as activity towards a substrate, of displayed variants of a parental molecule.

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences. In order to calculate the percentage identity between two nucleic sequences, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle (EMBOSS) or Stretcher (EMBOSS), the Smith-Waterman algorithm (e.g. as applied by Water (EMBOSS)), or the LALIGN application (e.g. as applied by Matcher (EMBOSS); and (ii) the parameters used by the alignment method, for example, local versus global alignment, the matrix used, and the parameters applied to gaps.

Having made the alignment, there are many different ways of calculating percentage identity between the two sequences. For example, one may divide the number of identities by: (i) the length of shortest sequence: (ii) the length of alignment: (iii) the mean length of sequence: (iv) the number of non-gap positions: or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the similarity or identity between two amino acid sequences may be calculated using the service Needle (EMBOSS) set to the default parameters, e.g. matrix (BLOSUM62), gap open (10), gap extend (0.5), end gap penalty (false), end gap open (10), and end gap extend (0.5). In another example, the similarity or identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle (EMBOSS) set to the default parameters, e.g. matrix (DNAfull), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (DNAfull), gap open (16), gap extend (4), alternative matches (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

Therapeutic antibodies have had a transformative clinical impact notably in inflammatory diseases and cancer, but their development remains time and cost intensive. Here we report deep screening, an ultra-high-throughput screening approach leveraging the Illumina HiSeq platform for parallel sequencing, display and rapid affinity screening at the level of >10⁸ individual antibody-antigen interactions. Deep screening enables the discovery of tens to hundreds of different low nanomolar to high picomolar nanobody (VHH) and single-chain Fv (scFv) antibody variants, both from yeast-display enriched VHH libraries as well as directly from unselected synthetic scFv repertoires. The large antibody-antigen interaction datasets produced by deep screening when combined with machine learning models enable in silico prediction of novel high-affinity scFv antibody sequences not present in the original repertoires. Deep screening promises to significantly accelerate the discovery of high-affinity antibodies for a wide range of targets.

Massively parallel assays provide the ability to enormously increase both the throughput and speed of data generation in the biomedical sciences. Comprising both repertoire selection approaches and direct biomolecular screening strategies they have proven key to the discovery of enzymatic catalysts and therapeutic antibody, peptide and small molecule drug leads for a number of disease targets.

While methods of diversification at the level of high-throughput DNA oligonucleotide synthesis is highly developed and various selection strategies (such as phage, yeast & ribosome display) are able to process and fractionate large combinatorial (poly) peptide repertoires (10¹⁰), these still only sample a fraction of the possible sequence space. Furthermore, all selection methods (to different degrees) suffer from inherent and inescapable biases due to varying levels of protein expression, display, folding efficiencies as well as potential toxicities to the host organism. Finally, such selections are generally conducted “in the blind” with little or no overall information on output until diversity has been sufficiently reduced to determine genotype emergence, abundance and enrichment by next generation sequencing (NGS).

Furthermore, even though NGS of selection repertoires can provide information on the distribution and enrichment of genotypes, multiple studies suggest that both genotype abundance and enrichment can be only weakly correlated to function (due to the aforementioned biases). Therefore, the genotype distribution obtained from sequencing data provides only an imperfect proxy for the global phenotypic and functional map of a particular biomolecular repertoire and thus does not significantly improve the discovery of highly functional but low abundance clones during a selection experiment.

Because of these shortcomings, as well as the desire of obtaining a more reliable global picture of genotype to phenotype correlations, numerous methods of high throughput screening have been developed. However, the majority of screening approaches are limited in scope, scale and information output. Isolated screening (one clone/compound/drug per well) does not easily scale, even with robotics, while for biologics, it is particularly difficult to determine the sequence composition of each well, and often only done for the identified hits. DNA, peptide and protein microarrays, where a known sequence is printed or synthesized in a defined position on a surface allow for the coupled measurement of sequence and function but tend to be limited in scale—with more than 500 k spots being prohibitively expensive for many labs.

A potentially transformative approach seeks to merge sequencing directly with functional screening. NGS technologies on the polony and Illumina platforms rely on extreme parallelization by sequencing clonal DNA from randomly arrayed DNA clusters. Both platforms have been leveraged to characterize DNA, RNA and polypeptides displayed on the post sequencing flow cell or captured within the polyacrylamide matrix. This has enabled the simultaneous interrogation of up to 2×10⁶ DNA- and RNA: protein as well as RNA:RNA and protein: protein interactions.

Here, we have sought to extend this concept to the powerful Illumina HiSeq platform with a potential diversity of up to 2×10⁹ clusters/interactions with a specific focus on antibody discovery. We demonstrate the display and screening of highly diverse, both pre-selected and unselected synthetic nanobody (VHH) and single-chain Fv (scFv) antibody libraries with the discovery of high-affinity (low nM to mid pM) binders directly from global equilibrium antigen binding data. Our approach, which we term deep screening, accelerates high-affinity antibody discovery from months to days. Furthermore, we demonstrate the utility of the large deep screening datasets for machine learning of antibody-antigen interaction parameters and the direct in silico prediction of high-affinity antibody hit sequences from antibody repertoire deep screening data.

### Example 1—Implementation of Ribosome Display and Deep Screening on a HiSeq 2500

Our ambition was to realize ultra-high-throughput antibody screening on the Illumina HiSeq sequencing platform, an approach we call “deep screening”. Several technical challenges needed to be overcome to achieve our aim, which are described below.

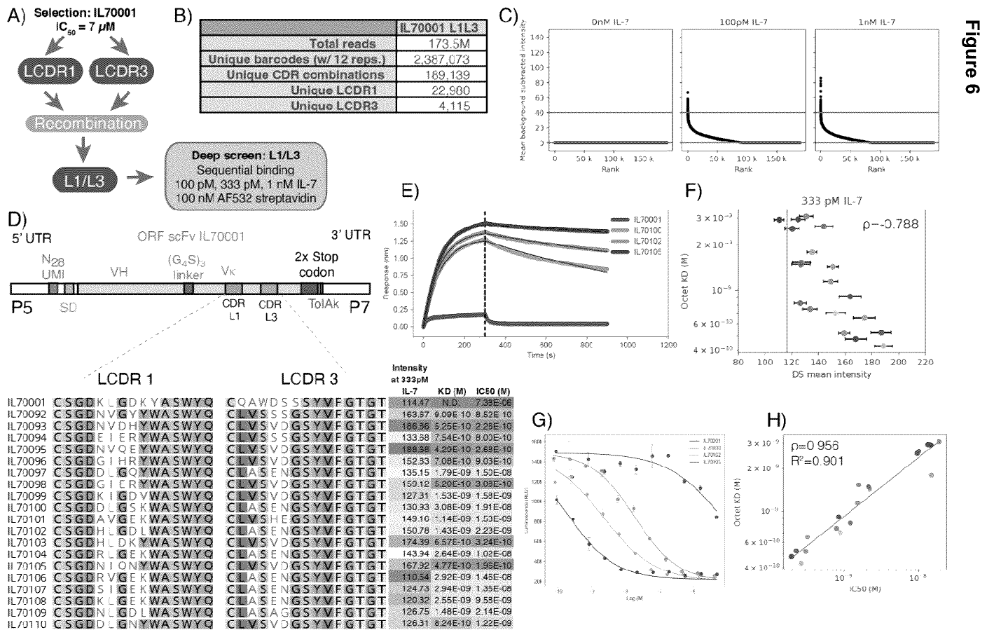

Illumina next generation sequencing operates on a highly integrated instrument with a flow cell comprising up to 2 billion (2×10⁹) clonal DNA clusters on the HiSeq 2500. These are generated in situ from individual, single-stranded (ss) DNA template molecules by a process called bridge amplification. Individual clusters typically comprise an array of ca. 1,000 DNA molecules in a ca. 1 μm diameter spot. Once arrayed, clusters are sequenced in parallel using Illumina's sequencing by synthesis (SBS) technology, yielding a large number of sequences and their physical x-y coordinates as an output. In order to implement screening of protein interactions, we first needed to develop methodologies to convert DNA clusters quantitatively into RNA and then protein clusters. To this end, we leveraged the efficient primer-dependent DNA-templated RNA polymerase activity of the engineered polymerase TGK to convert post-sequencing DNA clusters into RNA clusters. Specifically, we exploit the paired-end turnaround process to perform DNA bridge templated RNA synthesis (FIG. 4), whereby the surface-linked P5 primer is repeatedly extended by RNA synthesis on the DNA template. Once DTR is complete, the template is removed by restriction enzyme cleavage and DNA-RNA duplex denaturation at alkaline pH: creating ssRNA clusters covalently linked to the flow cell surface by the P5 primer (FIG. 4). These can be either interrogated directly or converted into peptide and protein cluster by in vitro translation (IVT).

Next, we developed a robust workflow to translate RNA clusters into polypeptides and stably display the resulting peptides or proteins on the flow cell surface. As 5′ tethered RNA clusters are vulnerable to nuclease degradation, we used the reconstituted PURExpress IVT system rather than more standard S30 IVT extracts, which can contain significant amounts of endo- and exonucleases. We specifically used PURExpress ΔRF123, −T7 RNAP, which lacks all release factors (RF-1, -2, -3) as well as T7 RNA polymerase in conjunction with an RNA construct that comprises the desired open reading frame (ORF) preceded by a 5′-UTR comprising a N28 unique molecular identifier (UMI/barcode), a translation initiation signal and followed by a 3′-extension sequence (to space out the ORF-encoded domain from the ribosomal exit tunnel) and two stop codons to stall the ribosomes (FIG. 4). Stalled mRNA: ribosome: nascent polypeptide complexes can be stabilized for several days at ambient temperature in high magnesium buffer, during which the flow cell array of up to several hundred million to several billion protein clusters with known sequences (or known unique molecular identifiers (UMIs)) can be interrogated for a variety of functional assays such as antigen binding.

Another technical challenge is presented by the nature of the HiSeq instrument, which is not designed for quantitative measurement; rather its imaging system is de-signed to threshold fluorescent intensity signals between four colour channels to determine base calls during sequencing. This poses challenges for quantitative measurement of binding interactions, which we solved algorithmically and experimentally by integrating equilibrium binding signal intensities at different concentrations with redundancy of each UMI. Furthermore, the HiSeq 2500 imaging platform utilizes an epi-fluorescent line scanning microscope with 532 nm and 660 nm lasers. The line scanning process of imaging a flow cell requires the instrument to detect a significant amount of illuminated signal in one of the 660 nm channels (as would be expected during a sequencing run) to first locate the flow cell surfaces and then maintain focus during a scan. This imaging mode is poorly suited for the screening of binding interactions, where clusters displaying a high signal are rare and do not provide sufficient signal for focusing. We solved this problem by labelling all RNA clusters through hybridisation of a fluorescently labelled DNA oligo to the 3′ end in the 660 nm channels, enabling focused imaging of the whole flow cell even with only sporadic or even no cluster signals in the 532 nm channels. In addition, this signal may serve as a diagnostic for RNA synthesis efficiency/cluster size and a normalization factor against the functional/protein binding signal from the same cluster. Finally, the ability to conduct all steps (comprising sequencing. RNA and protein synthesis and imaging) within the same instrument streamlines the experimental, imaging and data processing pipeline and avoids challenges with image alignment. Indeed, the HiSeq optical stage has outstanding x-y repeatability enabling efficient association of flow cell binding data with sequencing coordinates before quantifying fluorescence for each cluster (FIG. 4). Thus, using our custom image and data analysis pipeline, we are able to map barcode:phenotype pairs with barcode:genotype pairs, and recover a genotype:phenotype linkage for millions of clones.

Finally, cluster sizes and protein expression levels can be variable, which-together with other possible artefacts-introduces noise into the genotype: phenotype linkage datasets from deep screening. To correct for this inherent variability, we utilise redundant measurements of the binding signal from multiple clusters of the same barcode together with statistical outlier rejection to obtain reliable data. In the implementation described herein, we utilize the 2-lane HiSeq 2500 rapid run flow cell, with a maximum 3×10⁸ displayed clusters and aim for 12-fold redundancy. To achieve redundancy on the flow cell, libraries are bottlenecked between 0.1 and 1 fmol after attachment of UMIs. This yields a theoretical maximum diversity of 2.5×10⁷ UMIs,

The Deep Screening workflow thus proceeds in two phases. During the first phase, we sequence the N28 UMI barcodes for reasons of cost and time. RNA synthesis is then performed on the post-sequenced flow cell followed by in vitro translation (IVT) of the RNA clusters into protein clusters, which are interrogated for target binding in equilibrium binding and a kinetic dissociation assay. Binding and kinetic data is generated in the form of raw flow cell images, which are processed through our data analysis pipeline, which groups UMIs and equilibrium binding data, allowing for rapid verification of function within the library. If binding is observed, a second sequencing run is performed to sequence library members (fully or diversified segments thereof) and associate them with the N28 UMI barcodes, and thus binding data. Depending on the number and length of the variable regions to be sequenced, a deep screening experiment can be completed in as little as 3 days with data processing typically completed in several hours.

### Example 2—Identification of Rare, High Affinity Nanobodies Against Lysozyme

Having overcome the technical challenges associated with RNA cluster generation, protein display and imaging of the post-sequenced HiSeq flow cell, we first explored deep screening of a nanobody library. Nanobodies (VHH) are important tools in molecular and structural biology. We had obtained a commercially available yeast-display VHH library developed by the Kruse lab, with a reported diversity of 10⁸ on which we performed several (2-3) rounds of positive and negative magnetic-activated cell sorting (MACS) and fluorescence-activated cell sorting (FACS) selection for binding to a model antigen (hen egg lysozyme (HEL)) before deep screening the outputs on a flow cell for HEL binding (FIG. 5A). Upon processing the barcoded binding data, we identified 1,479 (MACS) and 3,687 (FACS) barcodes with mean integrated cluster fluorescence intensity values that were exceeding the background binding signal by at least a factor of two (at 300 nM HEL), indicating the presence of bona fide HEL binding VHH clones. To determine apparent HEL binding affinities (K_{D_app}), we performed an equilibrium binding affinity titration comprising escalating concentrations of HEL (1, 10, 100 and 300 nM). Binding at 300 nM HEL was followed a by measurement of dissociation rates (with the speed of the cluster signal dimming providing an apparent dissociation constant, k_{off_app}).

Next, we performed library sequencing to link the three CDR sequences (nanobody genotypes) to their equilibrium binding signals and dissociation rates (K_{D_app}, k_{off_app}) (nanobody phenotype), yielding 379,300 (MACS) and 39,900 (FACS) unique CDR combinations (FIG. 5B). Grouping binding data by unique CDR combinations yielded a total of 47 (MACS)/53 (FACS) putative VHH hits (FIG. 5C).

Deep screening datasets enable a global analysis of the antibody discovery process. For both MACS and FACS selection of yeast displayed VHH we observe a poor correlation between CDR abundance and high equilibrium binding signal (as a proxy for affinity) (Spearman rank correlation constant of ρ=0.361 (MACS), ρ=0.442 (FACS) at 300 nM HEL) (FIG. 5C). This suggests that in both the MACS and (to a lesser extent) FACS selection setups, inefficiently enriched high affinity clones, presumably due to strong biases (including toxicity, expression, folding or display efficiency variances) in the VHH yeast-display or biased amplification/transcription at the DNA/RNA level). Thus, while we were able to isolate rare binders from both R3 selections by deep screening (estimated hit frequency 0.049% (MACS) or 0.834% (FACS) (see above).

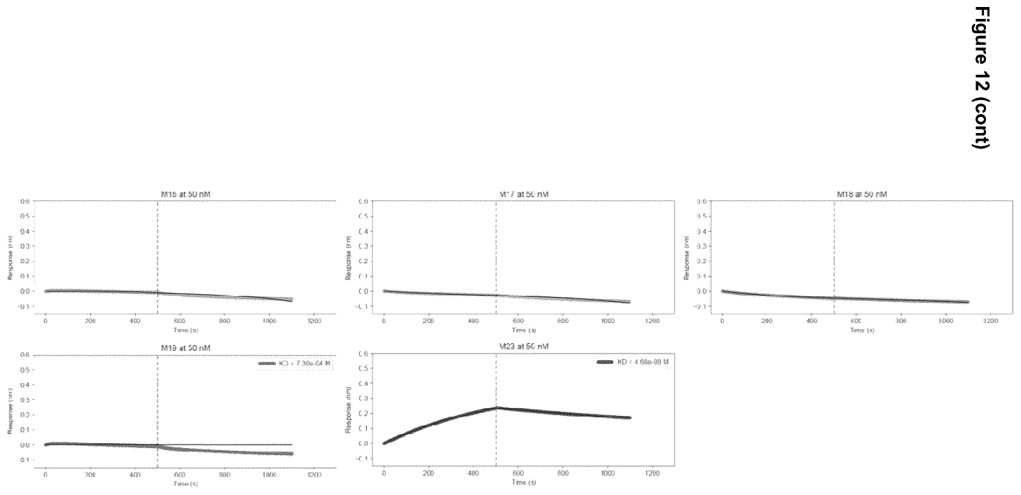

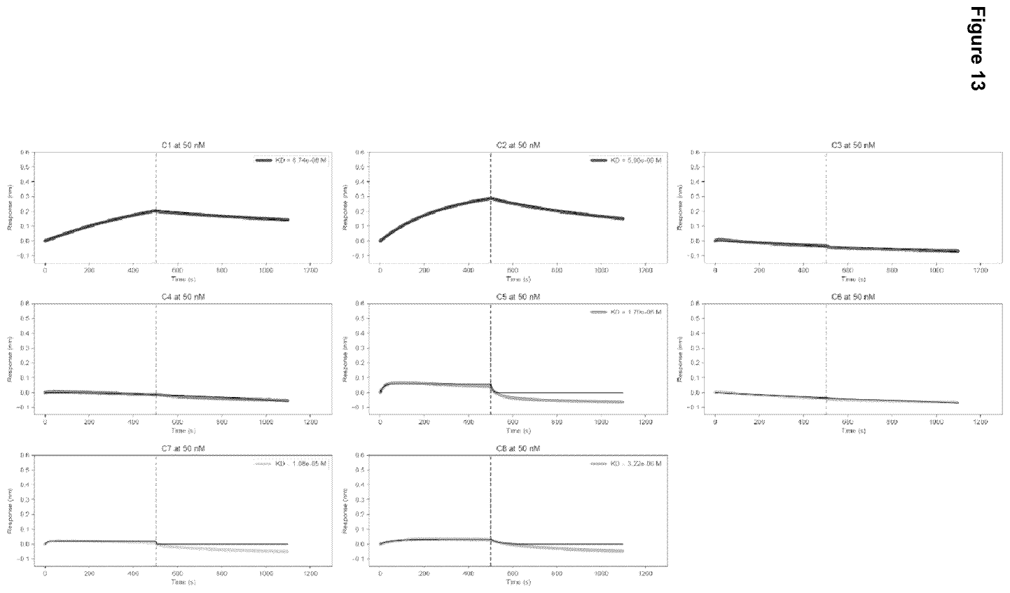

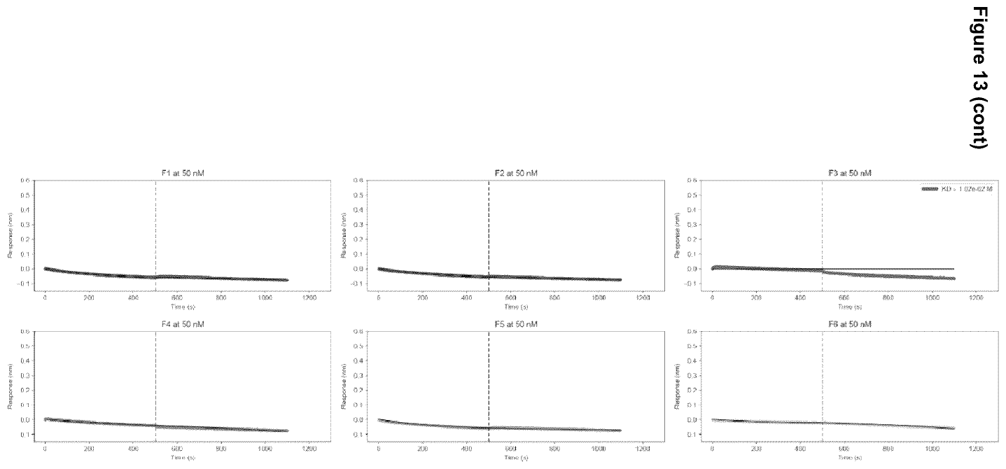

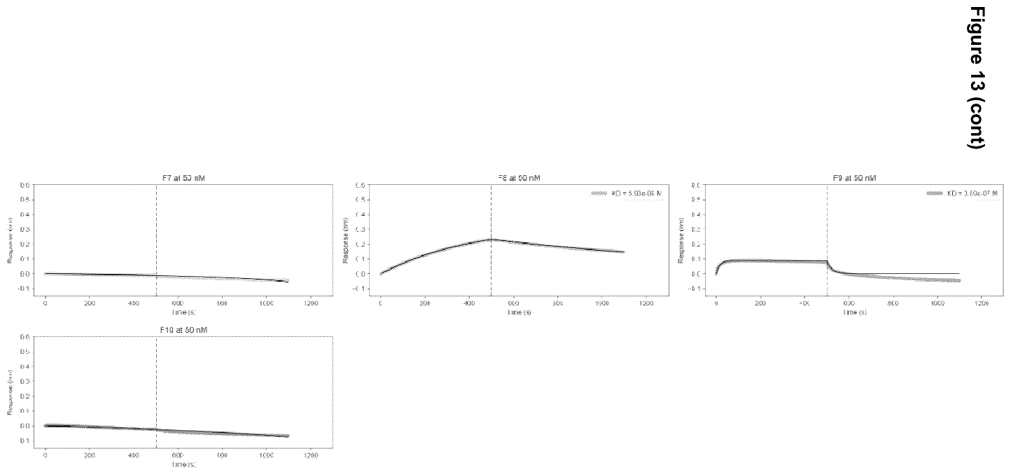

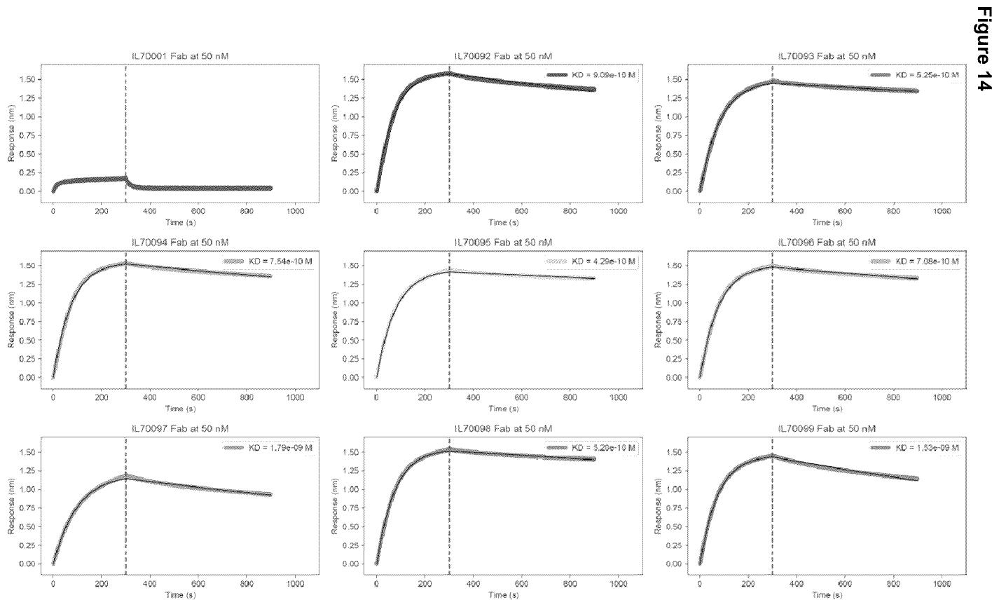

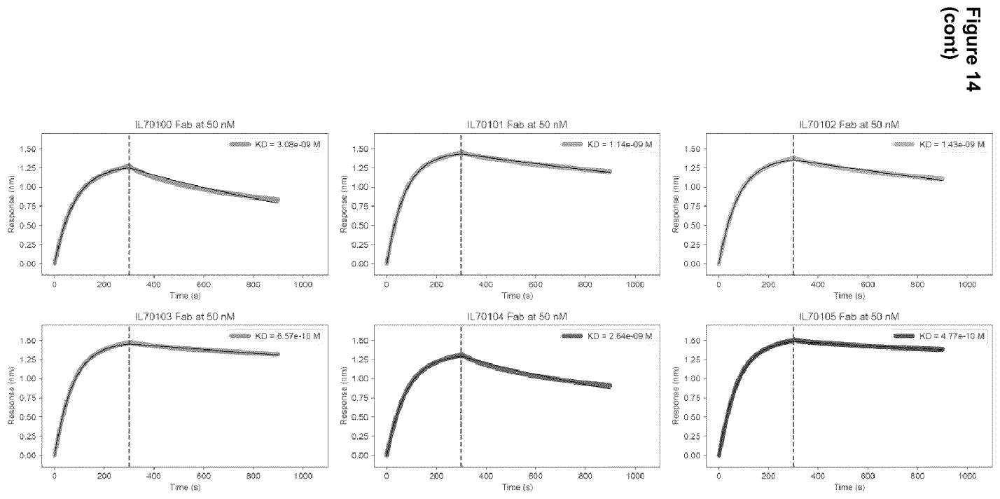

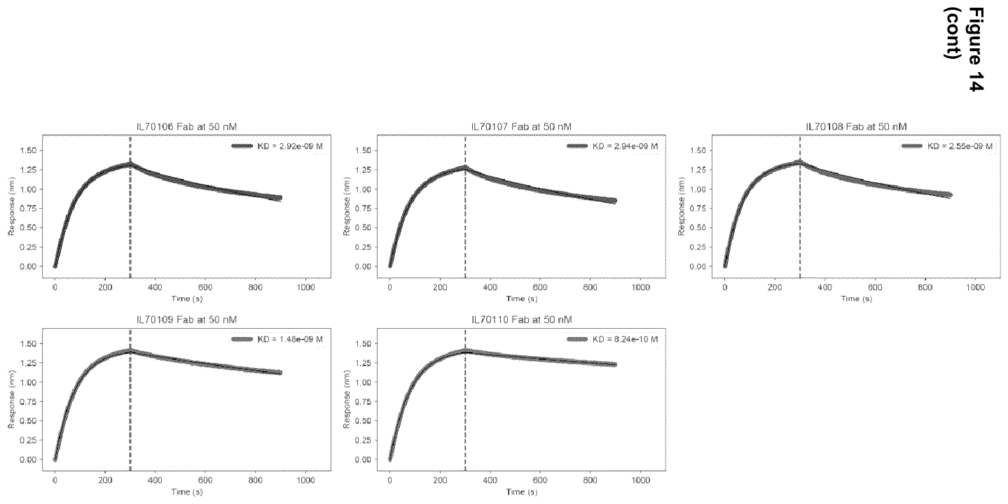

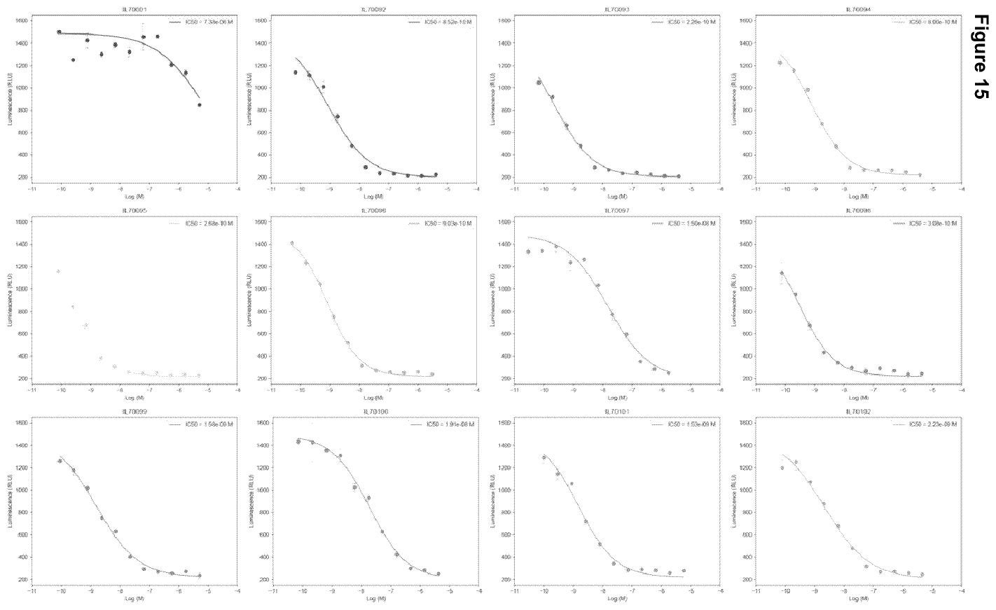

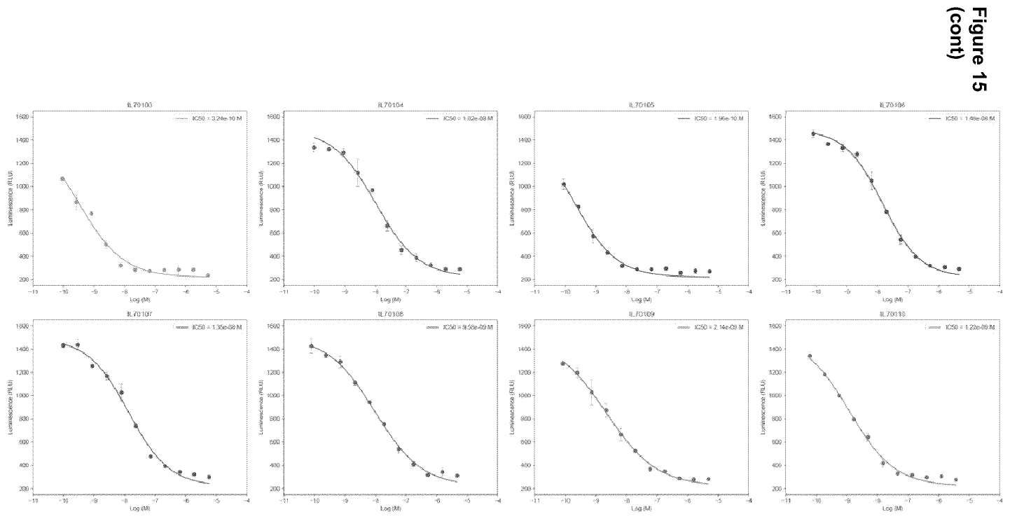

However, this conclusion rests on the conjecture that a high equilibrium binding signal (or equilibrium binding K_{D}: K_{D_app}) correlates with “true” high affinity binding (K_{D}) as measured by established biophysical techniques like biolayer interferometry (BLI). To evaluate this hypothesis, we chose 20 (M1-M19 and M23) and 10 clones (F1-F10) from the R3 MACS/FACS screens (respectively) with a wide range of observed fluorescent intensity, equilibrium binding signals and abundancies for characterisation (FIG. 5D). At the same time, we plated and picked 96 random colonies from the R3 MACS selection for colony PCR and Sanger sequencing. This identified 28 unique CDR sequences, 4 of which had already been selected for characterisation from the MACS/FACS libraries. We selected another 8 clones from the remaining 24 (C1-C8) for a total of 38 clones that were expressed and characterised for the measurement of binding kinetics by BioLayer Interferometry (BLI) (FIG. 5D-F, FIGS. 10-13). This identified three VHH hits with dissociation constants (K_{D}) of 9-20 nM ((M5 (1.9×10⁻⁸ M), M6 (1.42×10⁻⁸ M) and M15 (9.81×10⁻⁹ M)), and nine clones with lower K_{D}s ranging from 20-100 nM, including two from the randomly picked colonies (C1 and C2) (FIG. 5D, 5E). Plotting K_{D} values derived from BLI measurements against mean integrated intensities from deep screening reveals a Spearman rank correlation coefficient of ρ=−0.697 at 300 nM HEL (FIG. 5G).

While there are many potential factors that may explain the variances between deep screening and BLI, these results suggest that both nanobody abundance and enrichment can be only weakly correlated with affinity at least in some selection experiments. Despite using standard nanobody selection libraries and protocols, several more rounds would have been needed to further enrich the highest affinity library clones. Our results suggest that deep screening can cut this process short and discover high affinity binders (M5, M6, M15) even when still poorly enriched (with 3, 11 and 145 UMIs in 2.9×10⁶ screened). Indeed, identification of the same clones by standard procedures would have required the labour and time-intensive microplate expression and screening of tens of thousands of colonies.

### Example 3—Direct Affinity Maturation of an scFv Antibody without Selection

Having demonstrated the capacity of deep screening to identify low nanomolar binders from a pre-selected library, we sought to explore whether the discovery of high affinity antibodies is possible without any selection step, i.e. directly from a diversified repertoire of a low affinity parental clone.

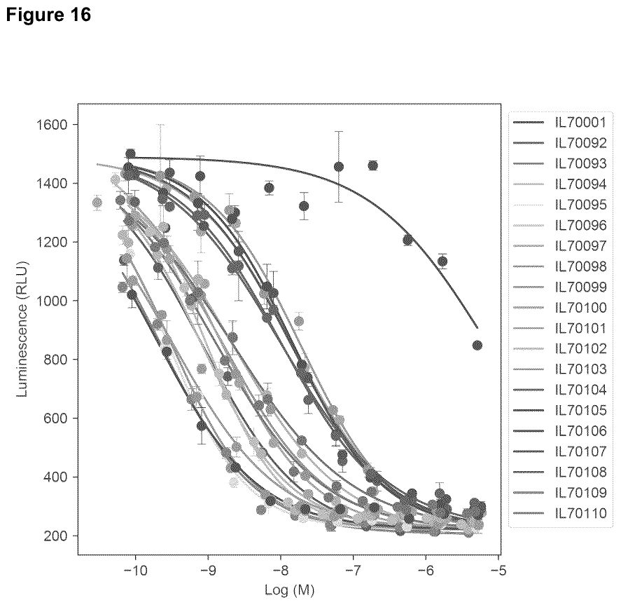

We started from a parental antibody, IL70001, which had been isolated by phage display from a human scFv library and determined to have a IC₅₀ of approximately 7 uM against human interleukin-7 (huIL-7) (FIG. 15-16)—a potential drug target implicated in multiple autoimmune and allergic inflammatory diseases. An affinity maturation library had been prepared by diversification of both Vk light chain CDR LI and L3 before we subjected this unselected library to direct ab initio deep screening (FIG. 6A).

Deep screening and CDR L1 and L3 sequencing yielded 1.7×10⁸ measurements comprising 2.4×10⁶ unique barcodes with >=12 replicates on the flow cell, and 1.9×10⁵ unique CDR combinations in protein space (FIG. 6B). Due to aggregation problems, we only collected binding data up to 1 nM of huIL-7, which revealed 173 unique, potential hits (FIG. 6C). Despite the large diversity of the input library, we observe a general convergence of CDR L3 loop sequences, while retaining significant diversity in the central region of CDR L1, presumably reflecting the larger contribution of CDR L3 to the IL-7 paratope. We therefore selected a subset (top 19 clones as judged by equilibrium binding signal at 1 nM huIL-7) plus IL70001 for in depth characterisation (FIG. 6D). We re-cloned these as Fab fragments to avoid potential pitfalls in affinity measurements due to scFv multimerization. Fabs were expressed and purified from CHO cells and binding kinetics were measured by BLI at 50 nM of each Fab, which revealed all 19 anti-IL-7 Fabs to have K_{D} values between 3 nM and 429 pM (FIG. 6D, 3E, S6). Since IL70001's K_{D} is significantly weaker than 50 nM, the maximum response measured and speed of the on and off rates is insufficient for an accurate fit of a dissociation constant (K_{D}).

IL-7's role in autoimmune and allergic inflammatory diseases depends on binding to the interleukin-7 receptor (IL7R). We therefore sought to assess whether our high affinity hits possess the ability to inhibit IL7 receptor (IL7R) signalling through the sequestration of IL7 using a TF-1 STAT5 IL7R alpha+gamma luciferase cell-based reporter assay. Indeed, we observe an average 10,000-fold increase in inhibition potency (IC₅₀) over IL70001, with IL70105 yielding a 37,000-fold improvement (FIG. 6G, FIG. 15, FIG. 16). We also observe a strong correlation between affinity and inhibition (ρ=0.956, R²=0.901 for fitting log(y)=m*log (x)+c) (FIG. 6H).

This data demonstrates that deep screening can rapidly identify multiple picomolar affinity antibodies directly from an unselected VL1/VL3 library against a therapeutically relevant drug target. We further observe a strong correlation between flow cell signal and BLI measured affinities (FIG. 6D, FIG. 6F, ρ=−0.788) even when switching antibody formats from scFv to Fab. The affinity increases from the parent antibody also deliver a four order of magnitude increase in inhibition potency of the target ligand. Bypassing selection in affinity maturation by deep screening delivers a major increase in discovery speed and provides a direct route to high affinity (pM) affinity antibodies from low affinity leads without the need for intermediate selection and screening processes. Further, isolated Fab clones exhibited favourable general properties and developability indicators such as good expression profiles (0.25-0.59 mg/ml of culture) and excellent monomericity (12/19 clones showed a >=98% monomeric fraction) as per HP-SEC (High Performance-Size Exclusion Chromatography).

### Example 4—Affinity Maturation of an Anti-Her2 scFv

Having demonstrated the ability to rapidly screen and identify high affinity nanobodies and scFvs from both selected and unselected libraries, we wanted to further explore whether the large and internally consistent deep screening datasets could be leveraged for supervised machine learning approaches to enable a more efficient exploration of CDR sequence space and discovery of high affinity antibodies.

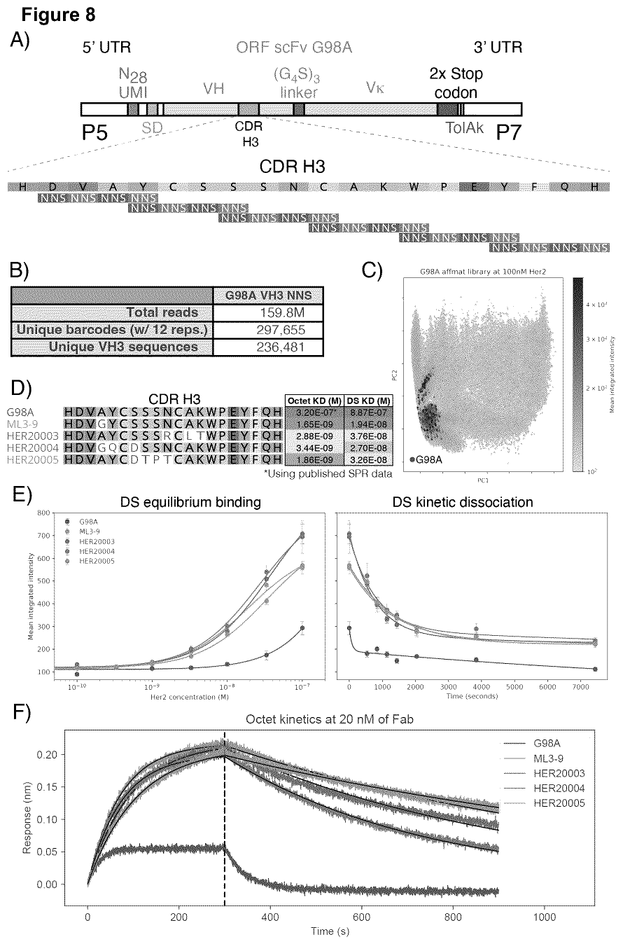

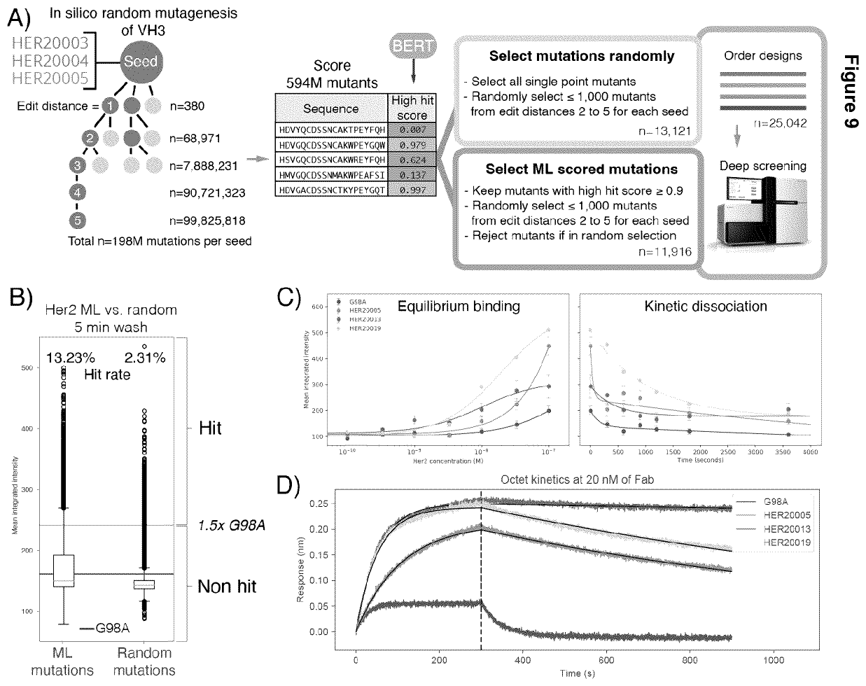

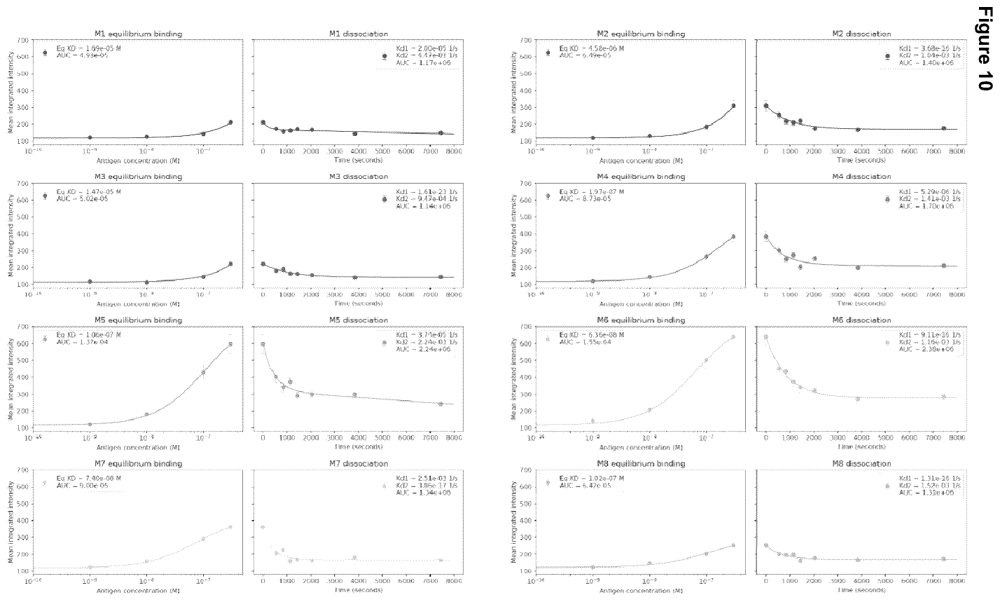

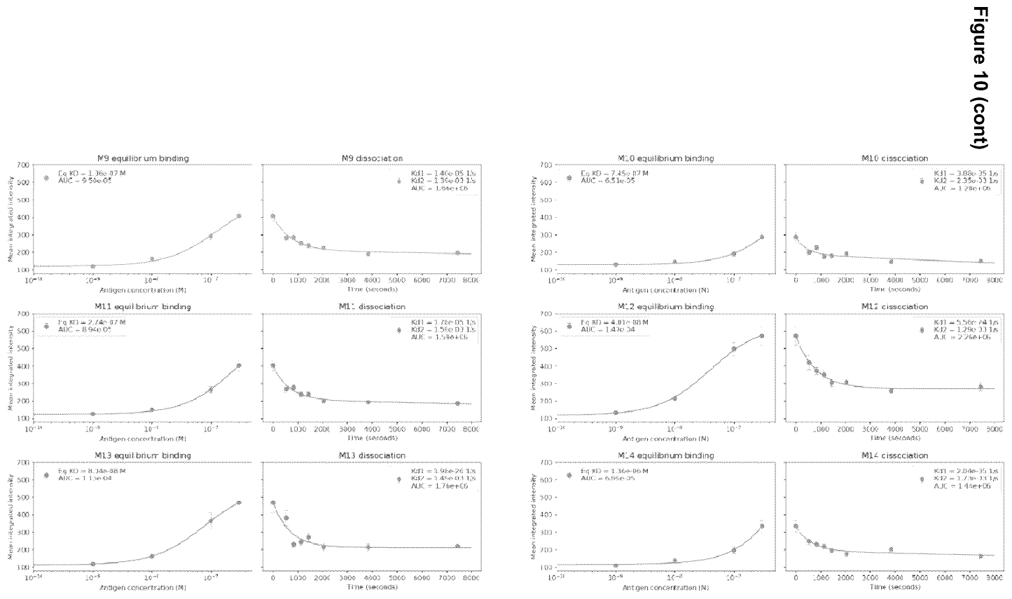

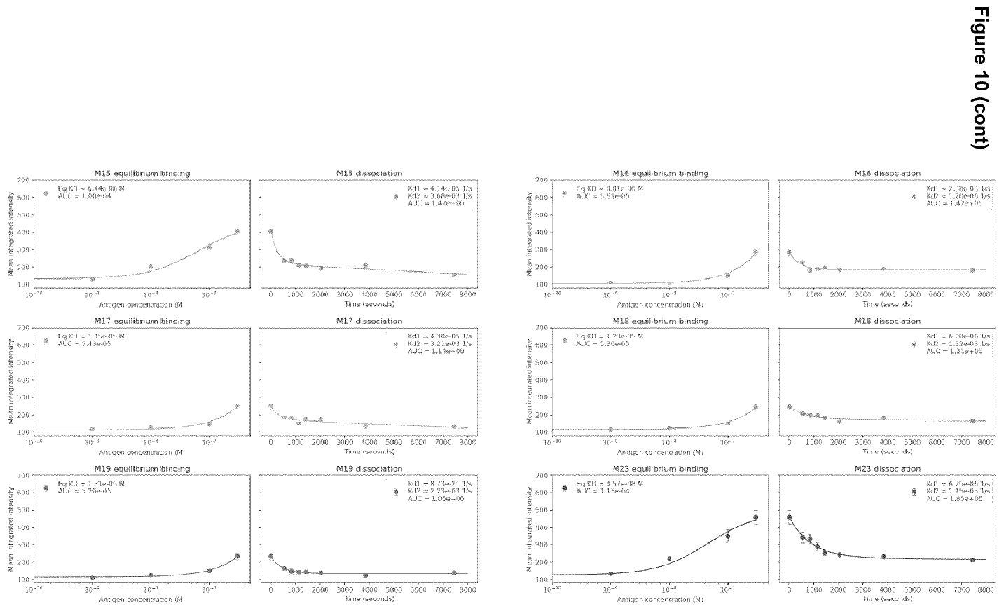

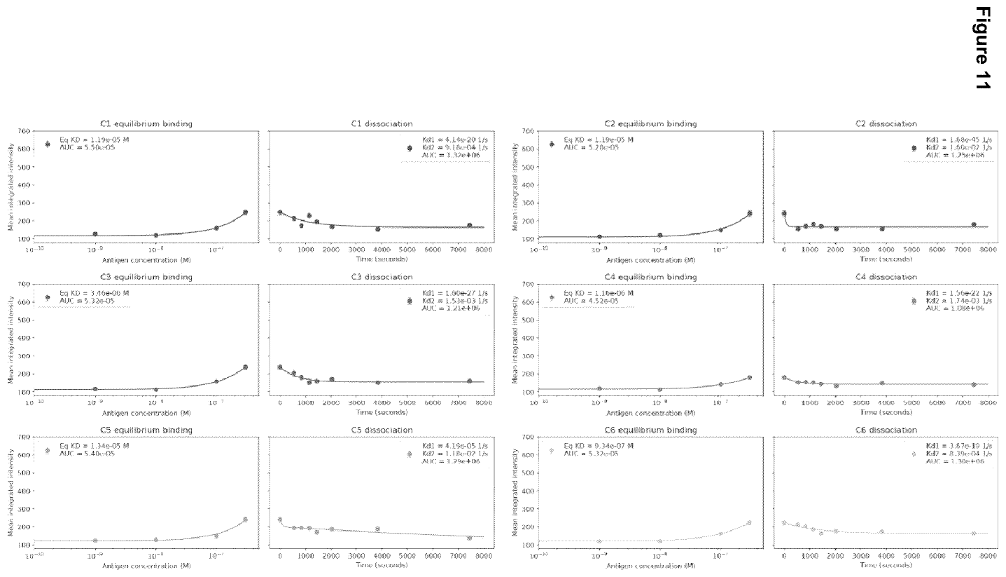

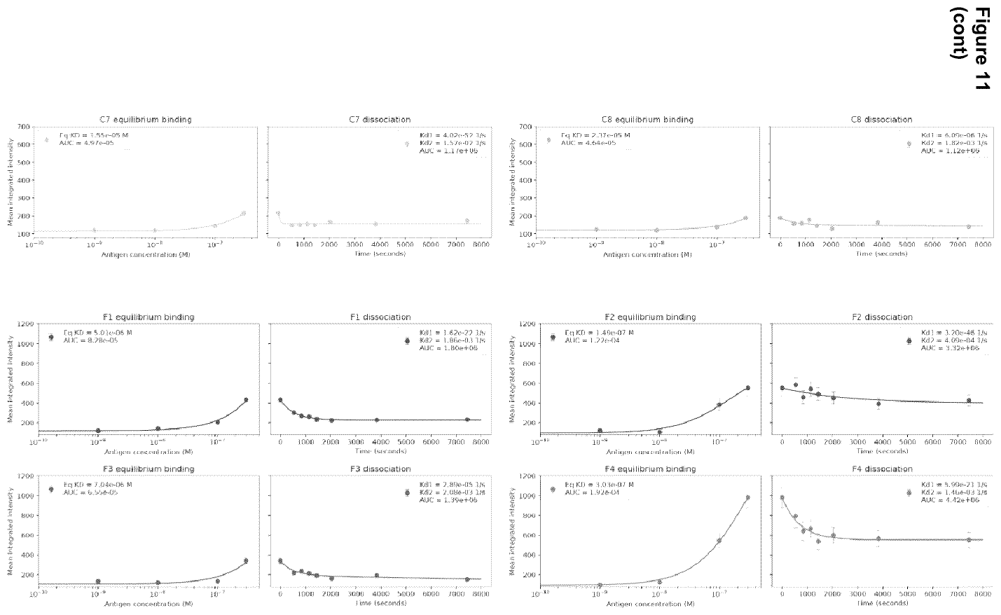

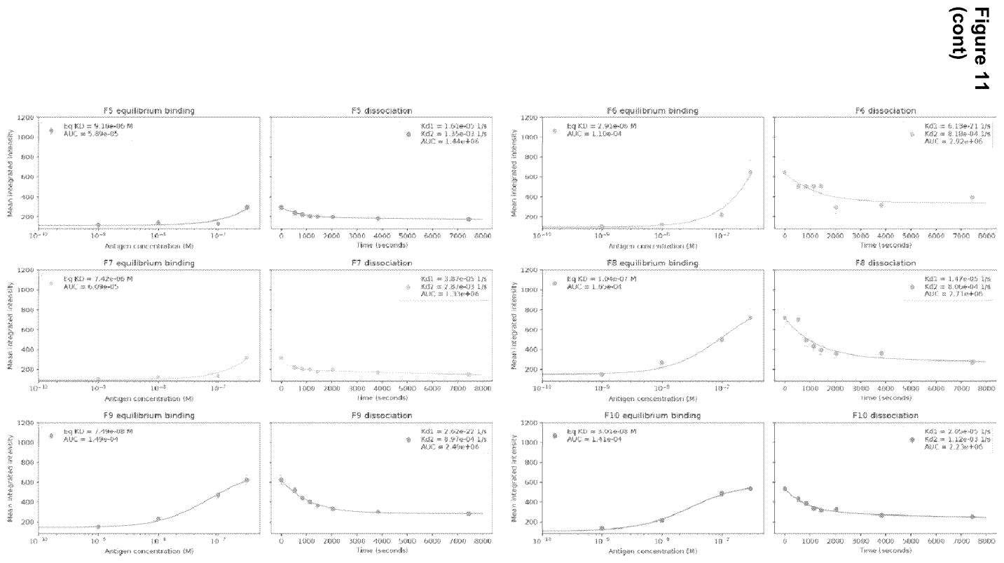

As a target, we chose HER2 (ERBB2), a cell surface protein tyrosine kinase over-expressed in 30% of breast, as well as ovarian, stomach and lung cancers. Her2 is the target of the highly effective therapeutic antibody trastuzumab (Herceptin), with a reported binding affinity of approximately 1 nM. We used a Herceptin scFv and a well characterized affinity panel of 5 scFvs (G98A, C6.5, ML3-9, H3B1 and B1D2+A1) with reported binding affinities (K_{D}) between 320 nM and 15 pM to benchmark our experiments (FIG. 8A). We first validated display of Herceptin and the anti-HER2 scFv affinity panel on a flow cell (FIG. 8B), which generally ranked the clones by equilibrium binding K_{D} and peak intensity (with the exception of B1D2+A1 and H3B1 appearing to be in the reverse ranked order) (FIG. 8C). Interestingly, the Herceptin scFv showed a significantly higher peak intensity relative to the affinity panel clones (FIG. 8C: with an equilibrium K_{D app} of 6.35 nM), presumably due to its well-known favourable expression, folding and stability.

With effective scFv display demonstrated, we chose the lowest affinity scFv G98A (K_{D})=320) nM from the affinity panel with barely detectable binding above background binding signal at 100 nM Her2 as a starting point for building an affinity maturation library. In doing so, we built six G98A CDR H3 scanning libraries of 4 NNS codons per window (FIG. 8A). On deep screening and subsequent CDR sequencing, we measured 2.98×10⁵ unique barcodes, coding for 2.4×10⁵ unique CDR H3 sequences in protein space (of a possible 6.2×10⁶) (FIG. 8B). While this sampled only a fraction of the potential CDR H3 library diversity, projecting VH3 sequence space into two dimensions with via principal component analysis (PCA) reveals that function is highly localised to three fitness peaks in local proximity to each other, and the majority of mutations sampled have no detectable binding at the highest concentration tested (100 nM Her2) (FIG. 8C). Inspection of the three highest scoring clones (peak intensity at 100 nM Her2 and in wash steps) yielded binding curves that closely match ML3-9 from the affinity panel with a known K_{D} of 1.0×10⁻⁹M (FIG. 8D, 8E, 17). Given the established correlation of flow cell data with affinity, these clones (HER20003, HER20004 and HER20005) likely have low nanomolar affinities for Her2, suggesting that deep screening alone had yielded 100-fold improvements to affinity. These three clones were subsequently converted into Fabs, expressed in CHO cells, purified, and binding kinetics characterised via Octet. On fitting a 1:1 model to the BLI data, we measured kinetic binding affinities of 2.8 nM. 3.4 nM and 1.8 nM for HER20003, HER20004 and HER20005 respectively, supporting the deep screening observations (FIG. 8D, 8F, 18). Although we expressed and purified G98A and ML3-9, a 1:1 model fit of G98A at 20 nM yielded a K_{D} (46.9 nM) that is 6.8× higher than the published literature. As the maximum response signal in BLI is rather low, we suspect that the specific binding is not well captured at this concentration of Fab. We will therefore refer to published literature K_{D} values for G98A (3.2×10⁻⁷ M).

Thus, deep screening was able to recapitulate phage-display affinity maturation of the anti-Her2 G98A scFv in a single three-day experiment. However, our motivation for this experiment had not been primarily affinity maturation, but rather the generation of a large dataset (‘HER2affmat’) linking CDR H3 sequence (genotype) to binding affinity (phenotype) (here comprising 2.4×10⁵ CDR H3 sequences) as an input for machine learning and in silico prediction of higher affinity Her2 binders.

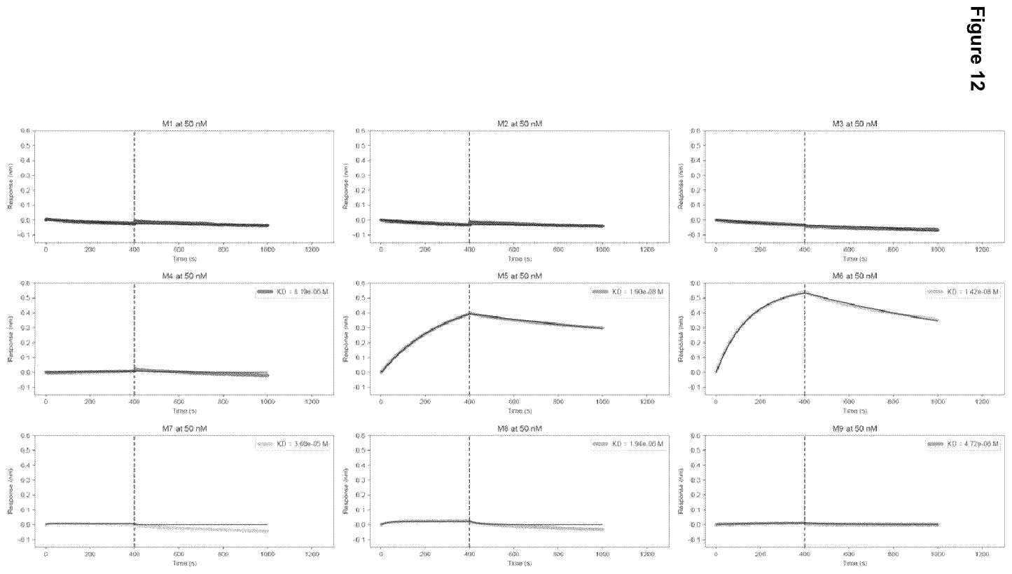

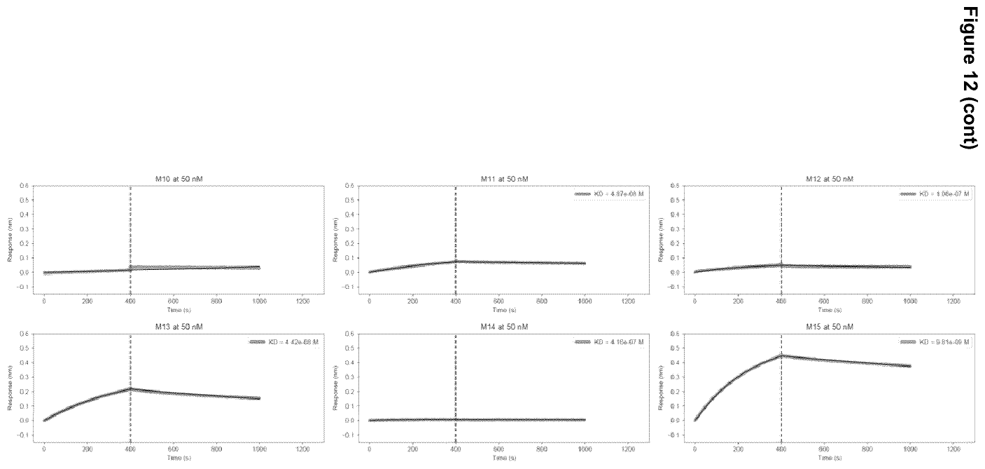

To this end, we built a machine learning model to predict Her2 binding by creating a classification problem, where predictions are binned into three categories (non-hit, low-hit, high-hit) by thresholding fluorescent intensity values from the 5-minute wash step (FIG. 9A). We selected threshold values such that the parent clone G98A was roughly centred in the low hit category, where G98A has an intensity of 190.05, and we chose a non-hit threshold of 150.0 and a high-hit threshold of 250; with the purpose being to balance the selection of enough lower nanomolar binders. With these thresholds, the “HER2affmat” data yielded 232,693 non-hit, 1,284 low-hit and 111 high-hit VH sequences. Our best model yielded acceptable performance metrics with F1-scores of 0.993, 0.329 and 0.480 for the non-hit, low hit, and high hit categories respectively (Table S2, S3). Although the F1-scores for the low and high hits were less than ideal, they were dominated by their high false positive rate, which is likely due to the challenge of defining the class boundaries across a continuous space of measurements.

With a trained model, we explored whether the model could be used to generate anti-Her2 binding sequences better than and more divergent from those observed in the “HER2affmat” dataset compared with random mutagenesis. To this end we took the three top scoring clones (seeds) from the “HER2affmat” dataset (HER20003, HER20004 and HER20005) and generated 1.98×10⁶ mutant VH3 sequences in silico for each seed (FIG. 9A). Specifically, we generated all single, double, and triple mutants and up to 10⁸ fourth and fifth order mutants randomly. All 594 million mutations were scored by the model before selections were made for a subsequent round of deep screening.

To compare the model against random mutagenesis, we devised a selection scheme where for each seed sequence a random mutation set was compiled from all single mutants and up to 1000 mutants from edit distances 2-5 yielding pool of 13,121 mutations (‘random/mut’). We next assembled a pool of sequences with exclusively machine learning generated mutations by removing all sequences with a high-hit score<0.9 and randomly selecting up to 1000 mutants from edit distances 2-5 as well as rejecting those that were already selected in the ‘random/mut’ set. This assembled a pool of 11,916 mutations (‘ml/mut’) (FIG. 9A).

Finally, we included clones G98A, ML3-9, HER20003, HER20004, and HER20005, which resulted in a total of 25,042 CDR VH3 sequences which we had synthesised as an oligonucleotide pool. On subsequent deep screening (with the same conditions as the “HER2affmat” library), we observed 24,968 of 25,037 clones from the designed library (99.72% coverage), and 174,700 extra mutants due to errors in array synthesis and cloning, for a total of 199,737 unique VH3 sequences in protein space. ML generated VH CDR3 sequences (‘ml/mut’) showed a striking improvement in fluorescent intensities with a significant upward shift in the distribution of high intensity clones in the 5 minute wash condition compared with random mutagenesis (‘random/mut’) (FIG. 9B), indicating that our machine learning model had been able to distil the salient features of high affinity Her2 binding from the “HER2affmat” dataset and use it to correctly predict a large number of novel Her2 binders.

As our aim was to leverage machine learning for the discovery of antibodies with higher affinities than the parental G98A, HER20003, HER20004, and HER20005 clones we explored the resulting deep screening data as a binary classification problem: with G98A now being centred in the non-hit category and clones with intensities 1.5× above G98A classified as hits (FIG. 9B). The resulting classification threshold revealed an overall hit performance of 13.23% for the ML clones (‘ml/mut’) vs. 2.31% for the randomly selected clones (‘random/mut’) (FIG. 9B, Table S4). Inspection of the number of hits per edit distance from the seed showed that the machine learning model improved sequence space sampling by 2.6× to 23.3× over random mutagenesis, with an average improvement of 5.7× (Table S4).

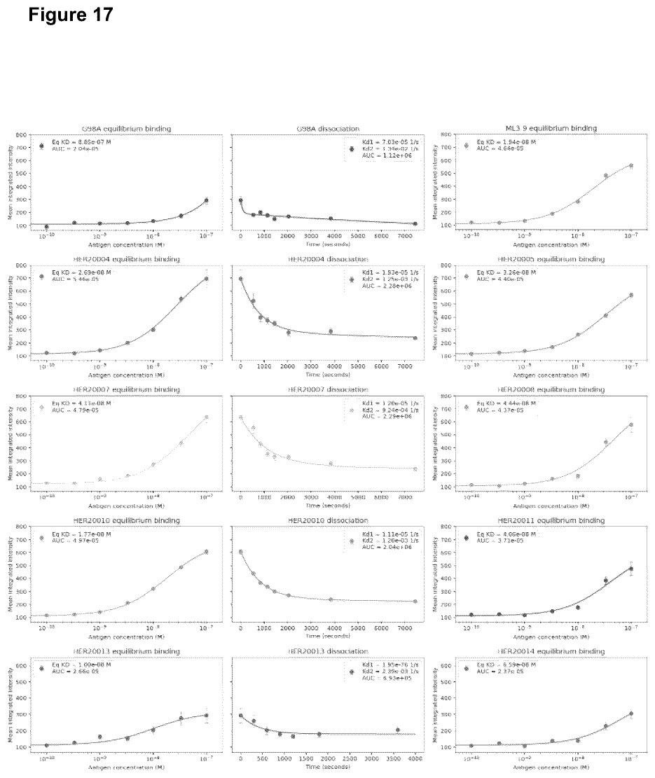

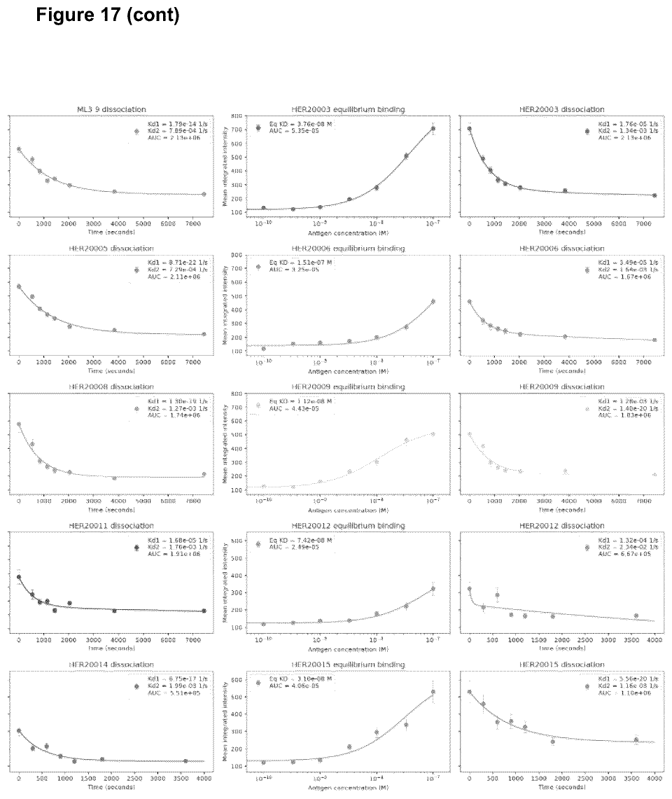

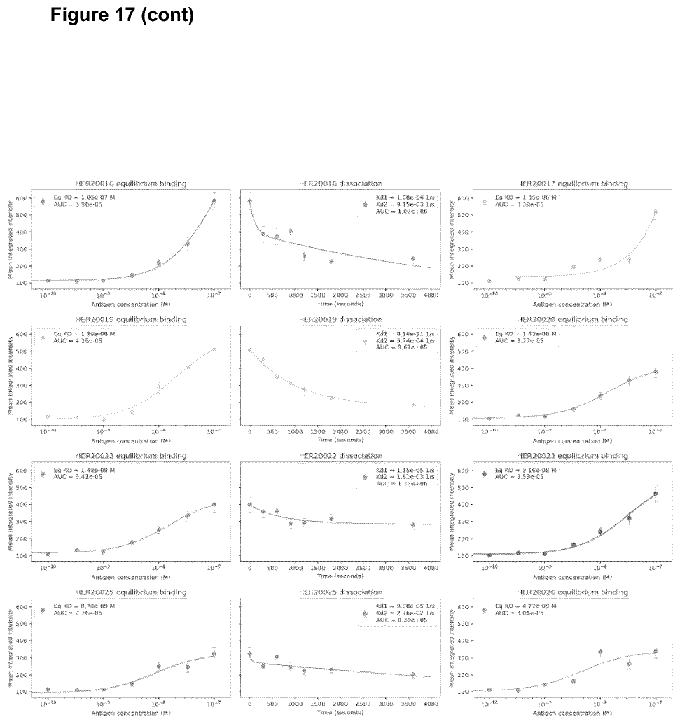

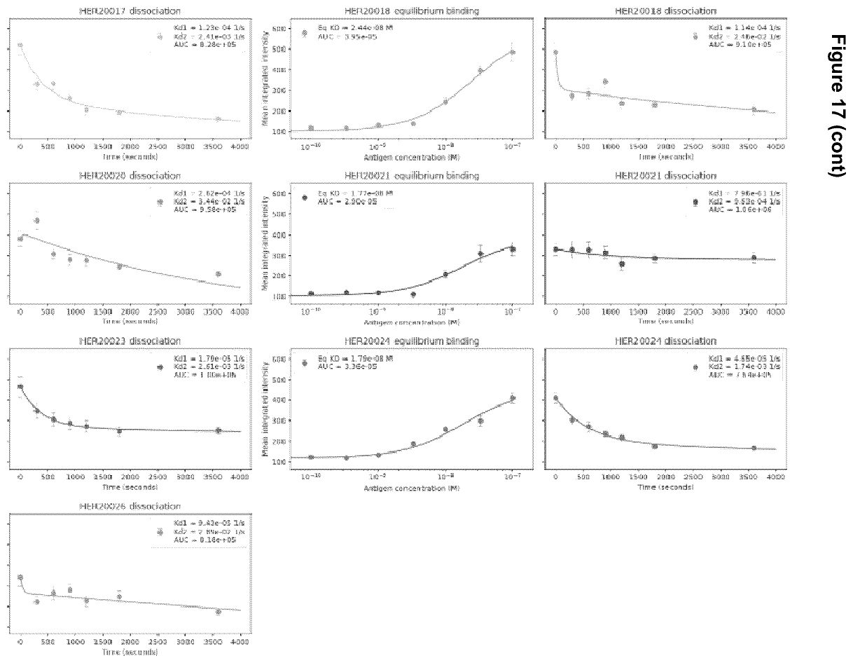

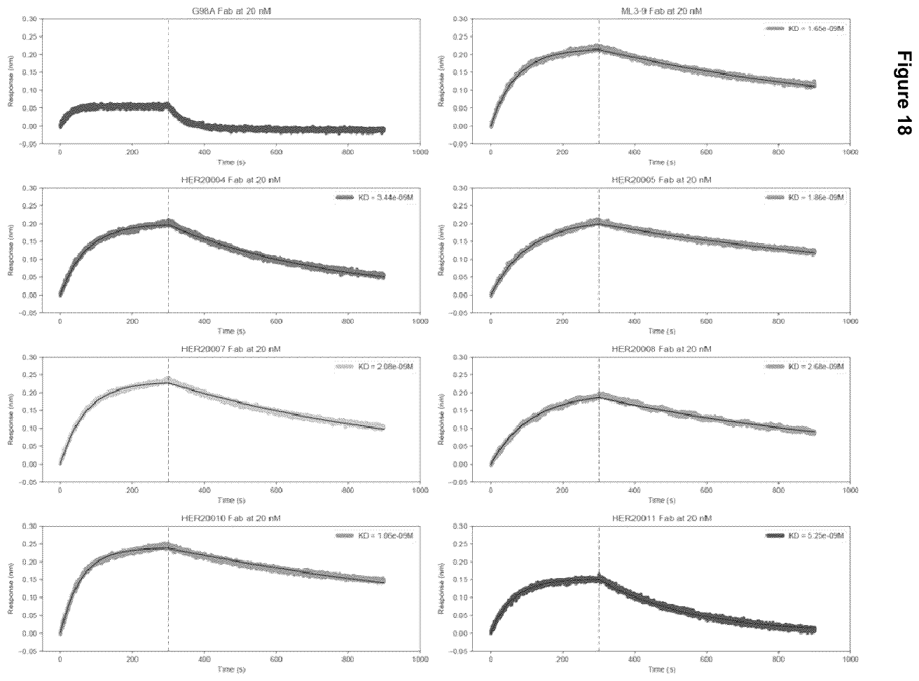

To determine affinities, we selected 21 new anti-Her2 scFv clones (6 from the “HER2affmat” library, 9 from the ML set (‘ml/mut’), and 6 from the random set (‘random/mut’)) for conversion to monovalent Fabs for expression in CHO cells, purification and characterisation (FIG. 17). These clones were selected based on a variety of criteria (peak fluorescent intensity at all concentrations, shape of the equilibrium binding curve, fluorescent intensity in wash conditions), to identify patterns that are more correlated with affinity, expression yield and monomericity.

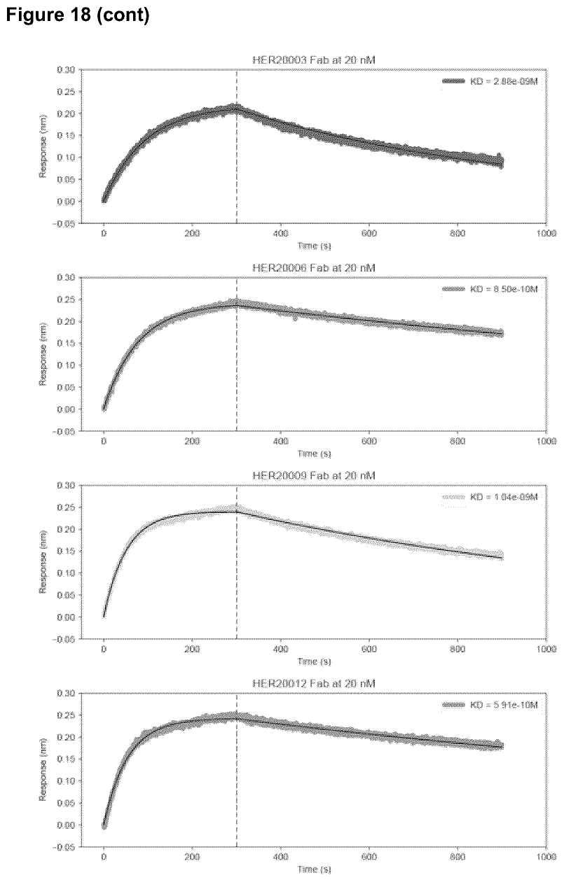

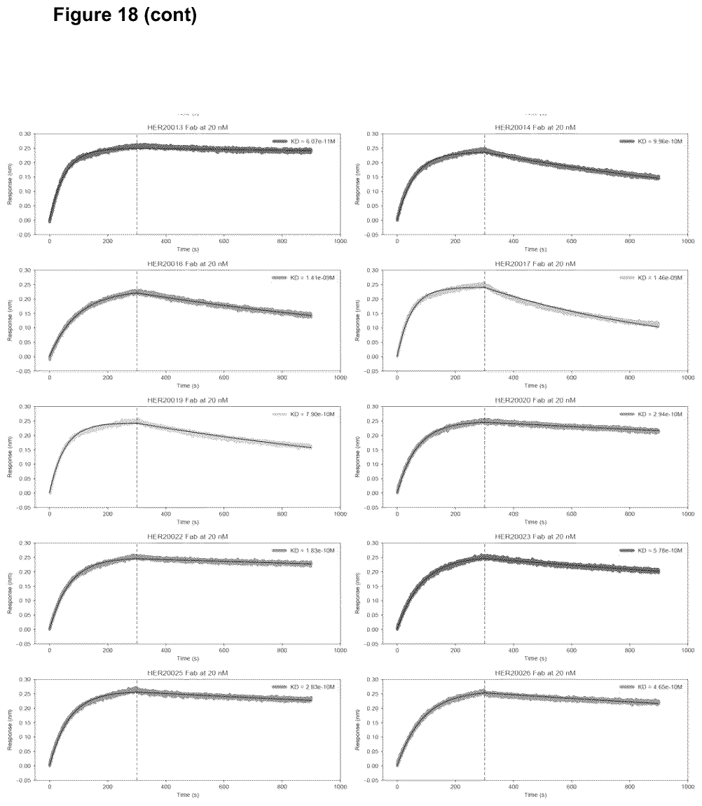

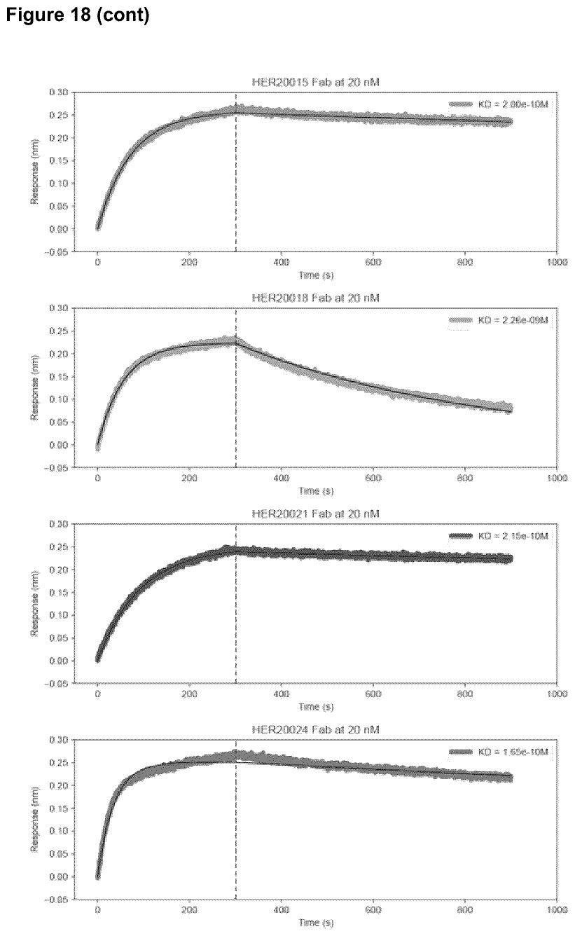

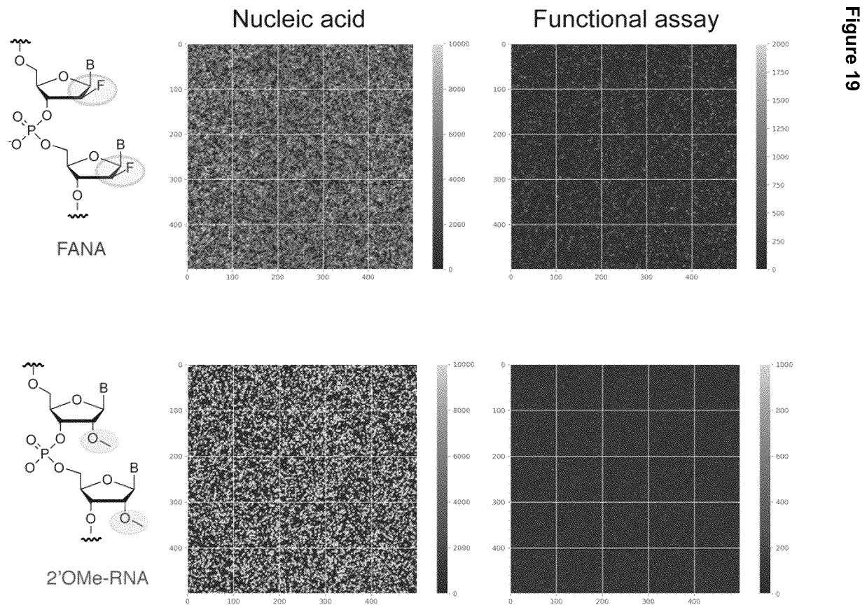

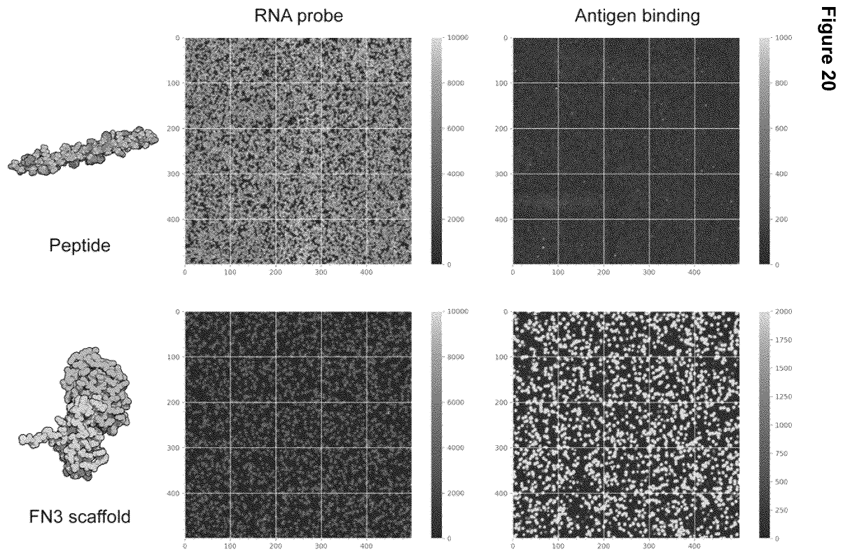

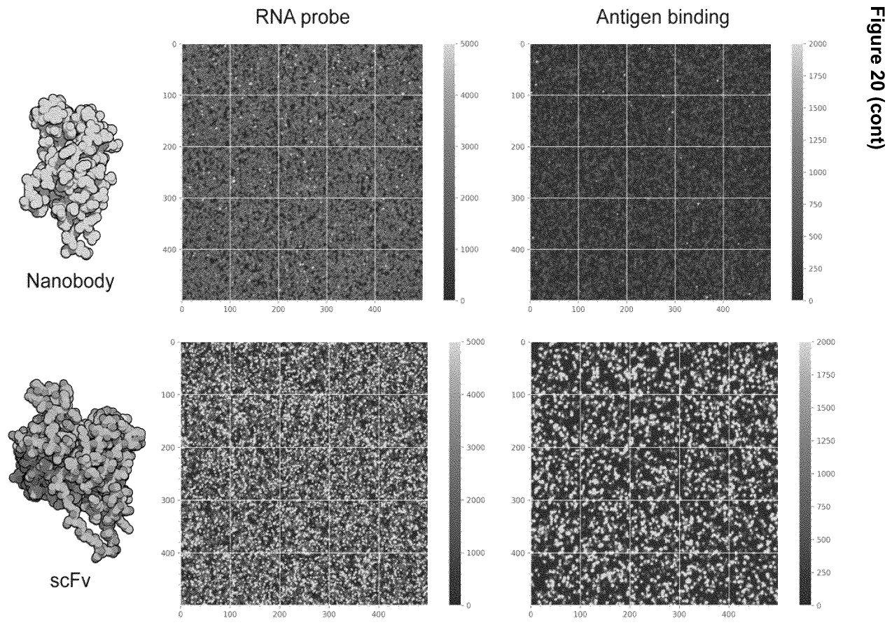

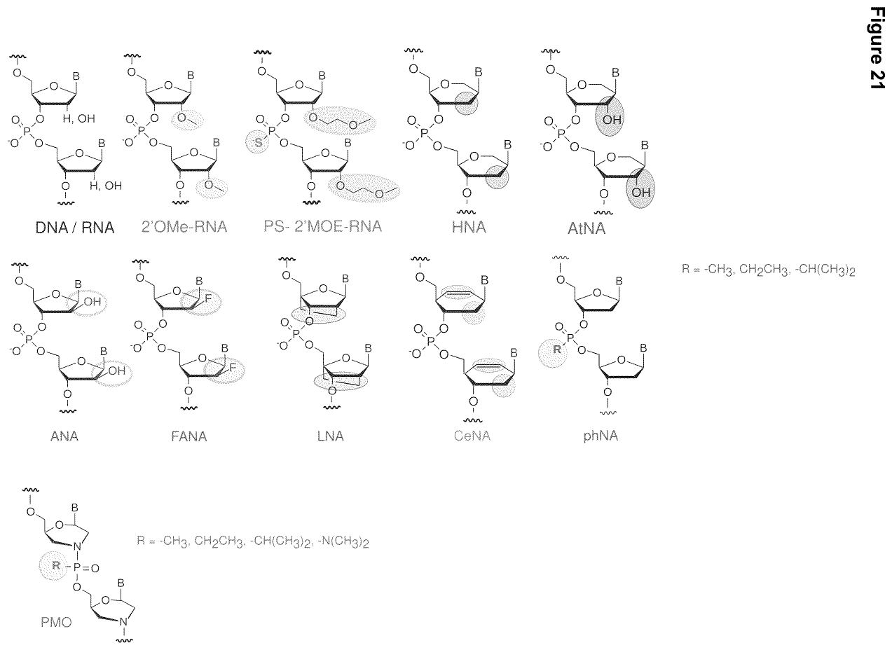

All of the selected clones derived from screening the “HER2affmat” library, including the three seeds (HER20003, HER20004, and HER20005) showed Kp values between 8.58×10⁻¹⁰ M and 5.25×10⁻⁹ M and a general improvement in monomericity (93.5% for G98A to 94.4-98.4% for the “HER2affmat” clones) (FIG. 18); and clones HER20006 and HER20010 showed a 300-fold improvement in affinity over G98A. Analysis of the clones present in the ML/random library indicated a further improvement of affinity (as per intensity values and binding curves), with the top clone from the ML set (HER20013) exhibiting a 5,220-fold improvement in affinity (K_{D}=6.07×10⁻¹¹ M) and another 4 clones (HER20015, 20, 21 and 22) from the ML set exhibiting a >1,000-fold improvement in affinity over G98A (FIG. 9C, 9D, FIG. 18).

While high intensity clones were 5× rarer in the random set, we still identified two clones (HER20024 and 25) which exhibited a >1,000-fold improvement in affinity over G98A (1.65×10⁻¹⁰ and 2.83×10⁻¹⁰ M respectively). In addition to affinity enhancement, we observed an overall improvement in monomericity for the ML and random clones over G98A monomericity from 93.5% to 98.1%; however, we were unable to identify any strong correlations between the deep screening data and monomericity. Taken together, these results demonstrate the exceptional effectiveness of combining deep screening with state-of-the-art machine learning models to discover high affinity antibody binders.

### Example 5—Display of an Anti-Her2 scFv Affinity Panel

In this example, we have clustered a library of anti-Her2 scFvs with a known affinity range of 3.2×10⁻⁷ to 1.5×10⁻¹¹ M on an Illumina flow cell using methods described wherein. We next sequenced 28 nucleotides, which resolved known unique barcodes for each clone and the spatial positions of every cluster on the flow cell surface. We then performed and validated RNA synthesis as described in the methods, before proceeding with in vitro translation and ribosome display. Ribosomes were stabilised with the addition of a buffer containing 50 mM MgAc, before being blocked with 0.1% BSA. A 0 nM control image was taken following a brief incubation of 100 nM AF532-streptavidin and buffer wash. We next performed an equilibrium binding affinity titration with 0.03 nM to 100 nM Her2-biotin and 100 nM AF532-streptavidin in a stepwise manner, saving images of the flow cell at each concentration: before measuring kinetic off-rates as described in the methods.

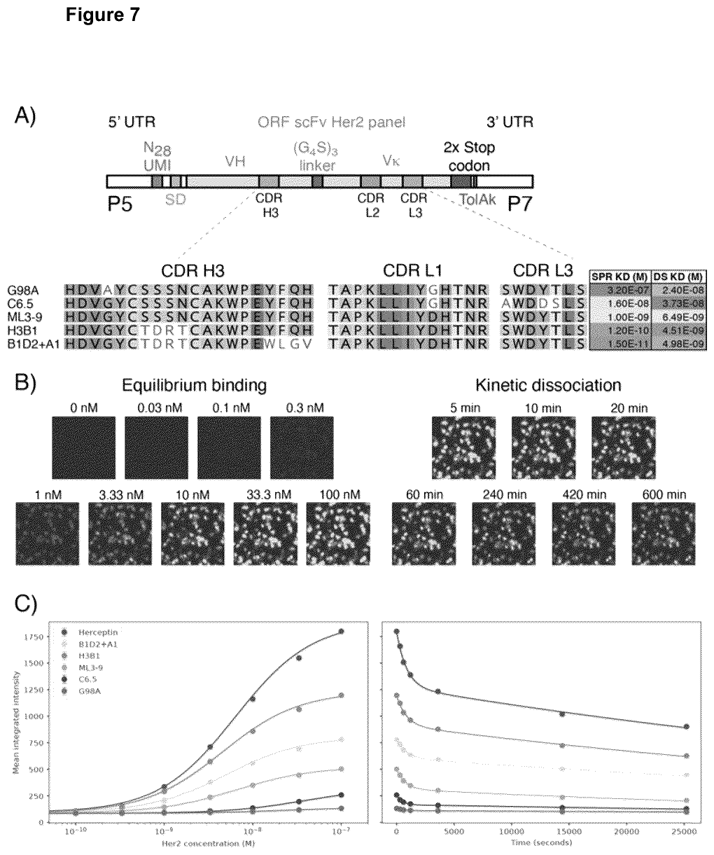

On processing the raw flow cell images through our data analysis pipeline, we were able to report mean, median and SEM (standard error of the mean) values for each clone at each concentration. Flow cell images throughout the experiment are shown in FIG. 7B, and median intensity values for each clone are reported for the equilibrium binding affinity and off-rate measurements in FIG. 7C. Curves were fit to these data as described in the methods, and resulting fits are presented in Table 4, along with SPR validated binding affinities against Her2.

TABLE 4

| | | Measured | Measured |
|---|---|---|---|
| | | equilibrium | dissociation |
| | SPR Kd | KD | rate constant |
| Clone | (M) | (M) | (k_{d}) (s⁻¹) |
| Her2_G98A | 3.2 × 10⁻⁷ | 1.62 × 10⁻⁸ | 5.67 × 10⁻⁴ |
| Her2_C6.5 | 1.6 × 10⁻⁸ | 3.45 × 10⁻⁸ | 2.30 × 10⁻³ |
| Her2_ML3-9 | 1.0 × 10⁻⁹ | 6.47 × 10⁻⁹ | 6.27 × 10⁻³ |
| Her2_H3B1 | 1.2 × 10⁻¹⁰ | 4.62 × 10⁻⁹ | 1.46 × 10⁻² |
| Her2_B1D2 + A1 | 1.5 × 10⁻¹¹ | 5.12 × 10⁻⁹ | 8.31 × 10⁻³ |
| Herceptin | 5.0 × 10⁻⁹ | 6.53 × 10⁻⁹ | 1.88 × 10⁻² |

Although fitted curves in this example do not perfectly match previously characterised results, likely due to incomplete saturation and differences between the methods, there are a few interesting observations to note in the data. In particular, the binding curve for Herceptin shows a slower equilibrium binding rate than H3B1 and BID2+A1, but a significantly higher Rmax. Since Herceptin is known in the literature to be a very well behaved scFv, in that it expresses and folds well, the significantly higher Rmax is likely due to a combination of binding affinity and expression/folding. Regardless, there is a clear rank order in the clones, which can be observed in the data, that enables the delineation of low nanomolar and high nanomolar binding affinities. This data therefore demonstrates the ability to display and measure equilibrium binding and off-rates of single chain antibodies on an Illumina flow cell.

### Example 6—Display of XNAs-2′-Fluoro-Arabinonucleic Acid (FANA)

Following sequencing we image the flow cell, which enables us to measure offsets and correct for chromatic aberration distortions between the different optical paths of the instrument. We then denature the sequencing product with a formamide wash at 65° C., followed by running Illumina's ‘End Deblock’ protocol, which uses reagents ‘Cleavage Reagent Mix (CRM) and Cleavage Wash Mix (CWM)’ to remove any remaining dye terminated nucleotides that are still present on the flow cell surface. With a single stranded DNA template present on the flow cell, we then need to ‘de-protect’ or remove the 3′ phosphate group from the P5 primer. This is done using the ‘Fast Resynthesis Mix (FRM)’ or T4 polynucleotide kinase (T4 PNK) and Illumina's de-protection protocol.

With a free 3′ hydroxyl group on the P5 grafted primer, we repurpose the paired end turn around process and perform a cycled RNA primer extension using D4YK polymerase. Here, D4YK will take a DNA primer (grafted P5) annealed to a DNA template and extend it with FANA ribonucleotides (faNTPs). This is done by heating the flow cell to 55° C. and performing 12 cycles of injecting denaturation mix, annealing and extension with 1×Thermopol buffer (20 mM Tris-HCl, 10 mM (NH₄)₂SO₄, 10 mM KCl, 2 mM MgSO₄, 0.1% Triton X-100, 200 uM faNTPs, 10 nM D4YK pol, pH 8.8): each extension step has an incubation time of 900 seconds.

Following 12 cycles of FANA extension, we anneal an oligo over the 8-oxoG site on the grafted P7 primer and perform cleavage (with Illumina's FLM2 reagent or 200 U/ml Fpg. 100 μl/ml BSA and 1×NEBuffer 1).

Following DNA cleavage and final extensions, we denature the DNA: FANA duplex and wash away the DNA template using a mixture of 100 mM NaOH and 5 mM EDTA, before cleaning the flow cell with 2 ml of 6 M GuHCI, 10 mM Tris, pH 7.4, and 2 ml of 5×SSC, 0.1% Tween 20. With clusters of single stranded FANA present on the flow cell, 100 nM of R2_atto647N and P7′_surface_hyb is annealed to the P7 adaptor at the 3′ end of each molecule of FANA.

### Example 7—Display of XNAs-2′-O-methyl Ribonucleic Acid (2′OMe)

Following sequencing we image the flow cell, which enables us to measure offsets and correct for chromatic aberration distortions between the different optical paths of the instrument. We then denature the sequencing product with a formamide wash at 65° C., followed by running Illumina's ‘End Deblock’ protocol, which uses reagents ‘Cleavage Reagent Mix (CRM) and Cleavage Wash Mix (CWM)’ to remove any remaining dye terminated nucleotides that are still present on the flow cell surface. With a single stranded DNA template present on the flow cell, we then need to ‘de-protect’ or remove the 3′ phosphate group from the P5 primer. This is done using the ‘Fast Resynthesis Mix (FRM)’ or T4 polynucleotide kinase (T4 PNK) and Illumina's de-protection protocol.

With a free 3′ hydroxyl group on the P5 grafted primer, we repurpose the paired end turn around process and perform a cycled RNA primer extension using 2M polymerase. Here, 2M will take a DNA primer (grafted P5) annealed to a DNA template and extend it with 2′O-methyl ribonucleotides (2′OMe NTPs). This is done by heating the flow cell to 55° C. and performing 12 cycles of injecting denaturation mix, annealing and extension with TAM (TGK Amplification Mix: 200 uM 2′OMe NTPs, 10 nM 2M pol, 2 M Betaine, 20 mM Tris, 10 mM Ammonium sulfate, 6 mM MgSO₄, 0.1% Triton-X, 1.3% DMSO, pH 8.8); each extension step has an incubation time of 3600 seconds.

Following 12 cycles of 2′OMe extension, we anneal an oligo over the 8-oxoG site on the grafted P7 primer and perform cleavage (with Illumina's FLM2 reagent or 200 U/ml Fpg, 100 μl/ml BSA and 1×NEBuffer 1).

Following DNA cleavage and final extensions, we denature the DNA: 2′OMe duplex and wash away the DNA template using a mixture of 100 mM NaOH and 5 mM EDTA, before cleaning the flow cell with 2 ml of 6 M GuHCI, 10 mM Tris, pH 7.4, and 2 ml of 5×SSC, 0.1% Tween 20. With clusters of single stranded 2′OMe present on the flow cell, 100 nM of R2_atto647N and P7′_surface_hyb is annealed to the P7 adaptor at the 3′ end of each molecule of 2′OMe.

### Example 8—Discussion

It has long been recognized that larger diverse repertoires of antibodies (and biomolecules in general) have a larger probability to contain a high-affinity binder as they cover the shape space of possible epitopes in a more complete manner. The experiments disclosed herein build on the pioneering work of many groups who have repurposed Illumina sequencing platforms for high-throughput screening by demonstrating the efficient display of single domain and single chain antibodies and also by extending the screening depth to 3×10⁸ on a 2-lane rapid run flow cell (and potentially to 2×10⁹ on an 8-lane flow cell). This has enabled the direct discovery of hundreds of different high affinity binders directly from unselected repertoires for two different human therapeutic targets (IL-7 and Her2) with affinity improvements of typically 2-3 orders of magnitude.

At this depth of screening, we can reveal the salient features of the antigen-binding paratope. For example, in the case of the IL-7 affinity maturation library, high affinity binders showed a high degree of convergence in CDR L3 sequence, while CDR LI remained more diverse although with signs of an emerging consensus sequence for the highest affinity clones. Likewise in the case of the Her2 affinity maturation library high affinity binders showed a degree of convergence around three core motifs.

A key observation from our experiments has been that “true” binding affinities as determined by state-of-the art biophysical measurements (Bio-Layer Interferometry, BLI) on individual purified, monovalent antibody Fab fragments (after conversion from an scFv), correlate well (ρ=−0.788 for the IL-7 clones) with the ranking and relative affinities as estimated by equilibrium antigen binding on the flow cell despite confounding factors such as potential avidity effects, differences in clustering and display efficiency, diffusion-related flow effects and the heterogeneous nature of the flow cell. Antibody ranking may be further improved and differentiated by utilising antigen dissociation kinetics, of which we have not in general exploited herein. In future, combining equilibrium binding and off rate measurements may allow the collection of global apparent affinity measurements across the whole displayed antibody library, which in turn provides a large, internally consistent dataset for machine learning guided sampling of CDR sequence space relevant to high affinity antigen binding with desirable binding kinetics.

We exemplified the utility for machine learning by training a state-of-the-art machine learning model to predict anti-Her2 binders using an experimental affinity maturation dataset generated by deep screening (FIG. 9A). We evaluated the ML model in a head-to-head comparison with random mutagenesis, where we computationally generated a set of 11,916 ML guided anti-Her2 mutants and a set of 13,121 random mutants, ordered this as an oligo pool and experimentally measured binding by deep screening. Here, we observed an overall 5× increase in the number of high intensity binders from the ML set compared with random mutagenesis (FIG. 9B), with the ML model yielding greater improvements over random as the number of mutations increased (23× at 5 mutations, Table S4). We characterised 15 high intensity clones (9 from the ML set, and 6 from the random set) and observed binding affinities between 2.26×10⁻⁹ and 6.07×10⁻¹¹ M, where the ML clone. HER200013, yielded a 5,200-fold improvement in binding affinity over the parental clone G98A (FIG. 9C, 9D).

While we are not the first to train machine learning models protein sequences and predict function, the majority of publications attempt to predict improvement to function that is encapsulated within the vast evolutionary history of a natural protein class. The engineering of binding to a specific antigen is substantially more challenging as the information does not exist within large sequencing datasets and any ML model relies on an antigen specific sequence to function dataset—of which deep screening can readily generate.

Intriguingly, antibodies isolated by deep screening not only display high affinity antigen binding properties but display other desirable “developability” features that are crucial for therapeutics, such as retention of affinity upon conversion to Fabs or IgGs, a high degree of monomericity and high expression yields in CHO cells. We hypothesize that these features arise due to a degree of pre-selection for desirable physicochemical properties by expression using a minimal translation apparatus (devoid of chaperones) and allowing expression and folding for 1 h at 37° C. scFvs that tend to misfold or aggregate would lead to a reduced equilibrium binding signal intensity and therefore be deselected.

The human immune system comprises ca. 10⁹ B-cells each displaying a different antibody and thus should be equipped to answer any antigenic challenge. In rodents, the immune repertoire is even smaller (10⁷), yet still antibodies to virtually any non-self-antigen can be raised. If naive repertoires could be faithfully displayed by deep screening a single repertoire might in principle yield binders to any desired target. However, in the immune system such early binders are usually of modest affinity (low micromolar to high nanomolar) with slow on rates and fast off rate kinetics, which are challenging to capture by the current implementation of deep screening. This is because imaging the flow cell (a 2-lane flow cell takes 4 minutes, and 16 minutes on an 8-lane flow cell) is often slower than the half-life of a low micromolar binder (a 1 μM affinity binder has a dissociation half-life of about 4-30 seconds). Nevertheless, further improvements in detection sensitivity (experimental and hardware) may in the future enable the screening of naïve libraries.

While Deep screening is currently implemented on a HiSeq 2500 platform, there are no obvious impediments to its extension to the more advanced HiSeq 4000 and NovaSeq platforms that are based on similar principles of clustering and imaging but use patterned flow cells rather than random clustering. It should also be noted that while we currently perform both sequencing and flow cell binding and imaging on the same instrument, external imaging is possible as demonstrated for the MiSeq platform and potentially would have advantages such as a wider range of colour channels and fluorescence imaging modes that could unlock the measurement of protein expression, non-specific and competition binding in the same assay.

In conclusion, deep screening expands the power of post sequencing screening to the HiSeq platform into the realm of hundreds of millions to billions of measurements. Together with methodological advances this allows for the display and direct screening of selected VHH and unselected scFv antibody libraries and the discovery of picomolar affinity binders from such libraries in days as opposed to weeks or months. Furthermore, the large, genotype-phenotype correlation datasets generated by deep screening allow efficient machine learning and sampling of antibody CDR sequence and antigen binding space yielding novel, higher affinity antibody sequences that were not present in the starting library. We anticipate many applications of the deep screening platform in particular in the discovery and development of therapeutic antibody drugs.

### Methods

### Construct Design

In order to transcribe and translate sequenced DNA clusters on an Illumina flow cell, our DNA constructs contained the following elements. A P5 adaptor, followed by a 28nt unique barcode, a 27 nt unstructured spacer (5p UNS v2), a ribosome binding site, start codon, protein coding region, TolAK short linker, 2× stop codons, a 27 nt unstructured spacer (3p UNS v2) and the P7 adaptor.

TABLE 1

| Construct element | Sequence |
|---|---|
| P5 adaptor | AATGATACGGCGACCACCGAGATCTACACTCTTTCCCTACACGACGCT |
| | CTTCCGATCT (SEQ ID NO: 5) |
| 5p UNS v2 | CATTACAAACGACACCCTAAACAAATC (SEQ ID NO: 6) |
| RBS | TATTTTAATAATTAAGGAGGTATATAC (SEQ ID NO: 7) |
| TolAK long linker | TATATGGCTAGTGGTGCCGAATTTGGGTCAGGTGGCCAGAAGCAAGCT |
| | GAGGAAGCCGCTGCCAAGGCTGCCGCAGATGCAAAGGCAAAAGCCGAG |
| | GCAGACGCTAAAGCTGCGGAAGAGGCTGCGAAAAAGGCAGCAGCCGAT |
| | GCTAAGAAAAAAGCGGAGGCGGAAGCCGCCAAAGCCGCAGCGGAAGCT |
| | CAGAAGAAGGCCGAGGCAGCTGCCGCGGCACTTAAAAAGAAAGCTGAA |
| | GCAGCAGAAGCGGCTGCGGCCGAGGCTCGTAAGAAGGCGGCAACAGAG |
| | (SEQ ID NO: 8) |
| TolAK short linker | TATATGGCTAGTGGTGCCGAATTTGGGTCAGGTGGCCAGAAGCAAGCT |
| | GAGGAAGCCGCTGCCAAGGCTGCCGCAGATGCAAAGGCAAAAGCCGAG |
| | GCAGACGCTAAAGCTGCGGAAGAGGCTGCGAAAAAGGCAGCAGCCGAT |
| | GCTAAGAAAAAAGCGGAGGCG (SEQ ID NO: 9) |
| 3p UNS v2 | TCCTGTTAGACTCCTCAATGCAAGCTG (SEQ ID NO: 10) |
| P7 adaptor | GATCGGAAGAGCACACGTCTGAACTCCAGTCACATCTCGTATGCCGTC |
| | TTCTGCTTG (SEQ ID NO: 11) |

Preparation of Anti-Her2 scFv Clones

Anti-Her2 scFv clones comprising Her2_G98A, Her2_C6.5, Her2_ML3-9, Her2_H3B1, Her2_B1D2+A1 and Herceptin are as disclosed in U.S. Pat. No. 8,580,263B2 and U.S. Pat. No. 5,772,997A. These clones span an affinity range from 3.2×10⁻⁷ to 1.5×10⁻¹¹ M and were designed to contain the construct elements as described above. The designed constructs were ordered as gBlocks from IDT (Integrated DNA Technologies), cloned into E. coli, and single colonies picked, validated by Sanger sequencing and extracted by PCR.

Linear double stranded DNA was diluted to 10 nM, and concentration quantified by qPCR with a KAPA Quant kit (KK4824, Roche).

TABLE 2

| Clone | Sequence |
|---|---|
| Her2_G98A | ATGCAGGTACAGCTTGTGCAGTCTGGGGCTGAAGTCAAGAAACCGGGTGAAAGTCTG |
| | AAAATTAGTTGTAAGGGCTCAGGATATTCGTTTACAAGCTATTGGATTGCGTGGGTT |
| | CGCCAGATGCCTGGTAAGGGATTAGAGTATATGGGCCTTATCTACCCGGGCGACTCC |
| | GATACCAAATACTCCCCAAGTTTTCAGGGTCAAGTAACCATTAGCGTGGACAAGTCT |
| | GTGTCCACCGCATACCTGCAGTGGTCGAGCTTGAAGCCCTCTGATTCTGCGGTATAC |
| | TTCTGTGCTCGTCATGACGTCGCCTATTGTTCCAGTAGCAATTGCGCAAAGTGGCCT |
| | GAGTATTTCCAACATTGGGGACAAGGGACCCTTGTCACAGTTAGTAGCGGGGGTGGT |
| | GGATCCGGCGGGGGCGGCTCAGGTGGAGGTGGAAGCCAGTCAGTATTAACTCAACCG |
| | CCTAGTGTCTCAGCAGCCCCGGGCCAGAAAGTTACGATTAGCTGTTCTGGATCAAGT |
| | TCAAACATCGGAAACAACTATGTGTCTTGGTATCAACAGCTGCCAGGGACAGCTCCT |
| | AAATTGCTGATCTATGGCCATACAAACCGCCCAGCAGGTGTACCCGACCGTTTCTCC |
| | GGATCGAAATCGGGTACATCCGCCTCGTTAGCTATTTCTGGGTTTCGCAGCGAAGAT |
| | GAAGCCGATTATTATTGTGCGAGTTGGGACTATACTCTTTCAGGGTGGGTGTTTGGT |
| | GGAGGGACGAAGCTTACTGTGCTTGGG (SEQ ID NO: 12) |
| Her2_C6.5 | ATGCAGGTGCAGTTGGTCCAATCAGGCGCCGAGGTAAAGAAGCCGGGGGAATCTCTT |
| | AAAATTTCCTGTAAGGGCAGTGGCTACTCCTTTACTAGTTATTGGATTGCCTGGGTT |
| | CGCCAAATGCCTGGGAAGGGTCTTGAGTATATGGGACTGATCTATCCCGGTGATTCT |
| | GACACCAAATACTCGCCAAGTTTCCAGGGACAAGTTACAATTTCTGTTGATAAGTCG |
| | GTATCTACCGCCTACTTGCAATGGTCATCCTTAAAACCATCCGACTCGGCGGTGTAC |
| | TTTTGTGCCCGCCATGATGTGGGGTACTGCTCGTCAAGCAACTGCGCGAAATGGCCC |
| | GAGTATTTTCAGCATTGGGGGCAGGGCACTTTAGTAACTGTTTCATCAGGTGGAGGA |
| | GGGAGCGGTGGAGGCGGGAGCGGTGGTGGCGGTTCTCAGTCTGTTTTAACTCAACCT |
| | CCGTCAGTTTCTGCTGCCCCAGGTCAAAAAGTCACTATCAGTTGTTCAGGATCGAGT |
| | TCGAACATTGGTAATAACTACGTATCCTGGTATCAACAATTACCCGGAACCGCCCCT |
| | AAGCTGTTAATCTATGGTCACACGAATCGCCCCGCCGGAGTTCCGGACCGCTTCAGC |
| | GGGAGTAAATCTGGAACGTCAGCCTCTCTGGCAATCTCAGGCTTTCGTTCAGAGGAC |
| | GAGGCTGACTACTATTGTGCGGCTTGGGACGACTCTCTTTCCGGCTGGGTCTTTGGT |
| | GGAGGTACAAAACTGACGGTGTTAGGT (SEQ ID NO: 13) |
| Her2_ML3-9 | ATGCAGGTCCAGCTTGTCCAGTCAGGTGCAGAGGTCAAGAAGCCTGGGGAGTCGCTG |
| | AAGATCTCATGCAAGGGGTCAGGATACAGTTTCACGAGTTATTGGATTGCATGGGTG |
| | CGTCAAATGCCAGGCAAAGGGCTGGAGTATATGGGGTTGATCTATCCGGGCGATAGC |
| | GATACGAAATACTCACCCAGTTTTCAGGGTCAAGTCACTATTTCGGTTGATAAATCC |
| | GTTAGTACTGCATACTTACAATGGAGCTCATTAAAACCCTCTGACTCGGCTGTCTAC |
| | TTTTGTGCCCGCCACGACGTGGGATATTGCTCGAGTTCTAATTGCGCGAAGTGGCCA |
| | GAGTATTTTCAGCACTGGGGGCAGGGGACACTGGTGACGGTAAGTTCAGGCGGGGGT |
| | GGGTCTGGTGGAGGAGGCTCTGGGGGAGGAGGATCTCAGTCTGTTTTGACGCAACCT |
| | CCCAGTGTGTCTGCCGCACCGGGTCAAAAGGTGACCATTTCGTGCTCCGGGTCATCT |
| | TCTAACATCGGGAACAATTATGTGTCCTGGTATCAGCAGCTTCCAGGGACTGCACCC |
| | AAACTGTTAATCTACGATCATACGAATCGTCCCGCTGGTGTGCCAGACCGCTTCTCT |
| | GGGAGCAAGTCAGGCACATCTGCATCCTTAGCCATTAGTGGCTTCCGCAGTGAAGAT |
| | GAAGCAGACTATTACTGTGCGAGTTGGGATTATACATTAAGCGGGTGGGTATTTGGA |
| | GGCGGCACAAAGTTAACAGTGTTAGGT (SEQ ID NO: 14) |
| Her2_H3B1 | ATGCAGGTGCAACTTGTCCAGAGCGGTGCTGAGGTAAAAAAACCTGGCGAATCTTTG |
| | AAAATCAGCTGTAAGGGCTCCGGTTACTCATTTACTTCATACTGGATTGCGTGGGTC |
| | CGCCAAATGCCTGGAAAGGGATTAGAATATATGGGGTTAATCTATCCAGGCGATTCA |
| | GACACGAAATATTCTCCAAGCTTTCAGGGTCAAGTCACCATCTCTGTCGACAAAAGC |
| | GTTTCGACAGCTTACCTGCAATGGTCTAGCCTGAAGCCCTCGGACAGTGCCGTATAC |
| | TTTTGTGCCCGTCATGACGTTGGATACTGTACTGACCGCACCTGTGCTAAATGGCCT |
| | GAATATTTCCAGCATTGGGGTCAGGGGACGTTAGTTACGGTCTCATCAGGTGGCGGA |
| | GGATCCGGTGGTGGTGGATCCGGTGGAGGGGGGTCACAATCCGTATTGACGCAGCCT |
| | CCCTCAGTAAGTGCAGCGCCTGGACAGAAAGTGACTATCTCTTGTTCCGGATCCTCT |
| | AGTAATATTGGGAATAATTACGTGAGTTGGTACCAGCAGCTGCCCGGCACCGCACCT |
| | AAGTTACTTATTTACGATCATACCAACCGTCCGGCGGGCGTACCAGACCGTTTCAGT |
| | GGCAGCAAGAGCGGAACCTCTGCGAGCTTAGCAATCAGTGGCTTTCGCTCAGAGGAC |
| | GAGGCAGATTATTACTGTGCATCATGGGATTATACTCTGTCAGGATGGGTTTTCGGC |
| | GGCGGGACAAAATTAACAGTCTTGGGT (SEQ ID NO: 15) |
| Her2_B1D2 + | ATGCAGGTACAATTGGTCCAGTCAGGCGCGGAGGTCAAAAAACCAGGGGAGTCTTTA |
| A1 | AAGATTTCGTGCAAAGGTTCCGGATATTCTTTCACTTCGTACTGGATCGCGTGGGTC |
| | CGCCAAATGCCAGGCAAGGGGTTAGAATATATGGGACTGATTTATCCGGGGGATTCG |
| | GACACCAAGTACTCCCCATCGTTCCAAGGACAAGTGACTATCAGCGTCGACAAATCT |
| | GTGTCGACCGCGTACTTGCAGTGGTCCAGCCTTAAGCCAAGTGACTCGGCCGTTTAC |
| | TTCTGTGCTCGCCATGACGTTGGGTACTGTACCGACCGTACATGCGCCAAATGGCCG |
| | GAGTGGCTTGGTGTTTGGGGGCAAGGGACTCTTGTTACTGTTTCTAGTGGAGGCGGG |
| | GGGTCAGGTGGGGGTGGCTCTGGTGGCGGGGGATCGCAATCAGTTTTGACTCAACCA |
| | CCTTCTGTTTCTGCCGCGCCCGGACAGAAGGTGACGATTAGCTGCAGCGGATCATCG |
| | TCAAACATCGGCAATAACTATGTTTCCTGGTATCAACAGTTGCCGGGAACCGCCCCC |
| | AAATTGTTGATCTATGACCATACTAACCGCCCCGCCGGAGTGCCTGATCGCTTCTCA |
| | GGGTCTAAGTCCGGCACGTCCGCAAGCTTGGCCATTTCCGGATTCCGCAGTGAGGAC |
| | GAAGCCGACTATTATTGCGCGTCTTGGGACTACACGCTTTCTGGATGGGTGTTTGGG |
| | GGAGGCACTAAACTGACCGTCCTTGGG (SEQ ID NO: 16) |
| Herceptin | ATGGAGGTTCAGCTGGTTGAAAGCGGTGGTGGTCTGGTTCAGCCTGGTGGTAGCCTG |
| | CGTCTGAGCTGTGCAGCAAGCGGTTTTAATATTAAAGATACCTATATTCATTGGGTG |
| | CGTCAGGCACCGGGTAAAGGTCTGGAATGGGTTGCACGTATTTATCCGACCAATGGT |
| | TATACCCGTTATGCAGATAGCGTGAAAGGTCGTTTTACCATTAGCGCAGATACCAGC |
| | AAAAATACCGCATATCTGCAGATGAATAGCCTGCGTGCAGAAGATACCGCAGTTTAT |
| | TATTGTAGCCGTTGGGGTGGTGATGGTTTTTATGCAATGGATGTTTGGGGTCAGGGC |
| | ACCCTGGTTACCGTTAGCAGTGGTGGTGGTGGTAGCGGTGGTGGCGGTTCTGGTGGC |
| | GGTGGTAGTACCGATATTCAGATGACCCAGAGCCCGAGCAGCCTGAGCGCAAGCGTT |
| | GGTGATCGTGTTACCATTACCTGTCGTGCAAGCCAGGATGTTAATACCGCAGTTGCA |
| | TGGTATCAGCAGAAACCGGGTAAAGCACCGAAACTGCTGATTTATAGCGCATCTTTT |
| | CTGGAAAGCGGTGTTCCGAGCCGTTTTAGCGGTAGCCGTAGCGGCACCGATTTTACC |
| | CTGACCATTAGCAGCCTGCAGCCGGAAGATTTTGCAACCTATTATTGTCAGCAGCAT |
| | TATACCACACCTCCGACCTTTGGCCAGGGCACCAAAGTTGAAATTAAA (SEQ ID |
| | NO: 17) |

### Cluster Generation and Barcode Sequencing

A library containing 5% of each of the above clones was clustered on an Illumina HiSeq 2500 using a paired end rapid run flow cell (PE-402-4002, HiSeq PE Rapid Cluster Kit v2, Illumina) at 6 pM, which typically results in ˜200 m reads. Although these flow cells are perfectly capable of being clustered to yield upwards of 400 m reads, in the downstream RNA synthesis and ribosome display steps, we chose to hybridise a fluorescent Atto 647N oligo to the P7 adaptor of each cluster to enable normalisation of the binding assay. At densities higher than 200 m reads, our HiSeq 2500 is unable to reliably focus and image the flow cell with all the RNA clusters labelled. Use of a fiducial would enable higher cluster densities, but would result in no information about RNA synthesis efficiency.

We modified the standard Illumina DNA cluster generation protocol to increase the number of bridge amplification cycles from 28 to 32, and added a 60s wait time to each amplification cycle. Further modifications were made to the amplification mix, which comprises 2M Betaine, 20 mM Tris, 10 mM Ammonium sulfate, 6 mM MgSO₄, 0.1% Triton-X, 1.3% DMSO, 200 μM dNTPs, 80 U/ml Bst 2.0, pH 8.8, and the denaturation mix, which comprises 98% formamide, 10 mM NaOH, and 1 mM EDTA. We found the combination of these modifications to greatly improve the signal of clusters grown from long templates such as single chain antibodies, which can be upwards of 1.2 kb.

Clustering and sequencing was performed as a paired end, single read run with no indexing for 28 cycles on read 1, and 0 cycles on read 2, and executed using the HiSeq Control Software (HCS v. 2.2.68, Illumina). The flow cell and clustering reagents are sourced from the HiSeq PE Rapid Cluster Kit v2 (PE-402-4002, Illumina) and sequencing reagents were sourced from the HiSeq Rapid SBS Kit v2 (FC-402-4023, Illumina).

### RNA Synthesis

Following sequencing we image the flow cell, which enables us to measure offsets and correct for chromatic aberration distortions between the different optical paths of the instrument. We close HCS and launch the HiSeq engineering software (Archimedes Test Software v. 3.8.317.0, Illumina), initialise the instrument, home the stage, set the chemistry module run mode to ‘RapidRun’ and set the flow cell temperate to 20° C. We then pump 120 μl of Illumina's universal sequencing buffer (USB) into the flow cell before auto tilting, aligning and imaging the flow cell using the ‘Bruno Scan’ module. We do this specifically by setting the Surface to ‘Dual Lane’, the Scan Velocity to 2.0 mm/s and the Swath to ‘Dual Swath’. The flow cell images are saved and enable us to measure offsets and chromatic aberration distortions between the different optical paths of the instrument.

We then denature the sequencing product with a formamide wash (e.g. FDR-Illumina's ‘Fast Denaturation Reagent’) at 65 C, followed by running Illumina's ‘End Deblock’ protocol, which uses reagents ‘Cleavage Reagent Mix (CRM) and Cleavage Wash Mix (CWM)’ to remove any remaining dye terminated nucleotides that are still present on the flow cell surface. With a single stranded DNA template present on the flow cell, we then need to ‘de-protect’ or remove the 3′ phosphate group from the P5 primer. This is done using the ‘Fast Resynthesis Mix (FRM)’ or T4 polynucleotide kinase (T4 PNK) and Illumina's de-protection protocol.

With a free 3′ hydroxyl group on the P5 grafted primer, we repurpose the paired end turn around process and perform a cycled RNA primer extension using a TGK polymerase. Here, TGK will take a DNA primer (grafted P5) annealed to a DNA template (cluster strands) and extend the primer with ribonucleotides (NTPs). This is done by heating the flow cell to 55° C. and performing 12 cycles of injecting denaturation mix (FDR), annealing and extension with TAM (TGK Amplification Mix: 625 μM NTPs, 10 nM TGK, 18 U/ml Superase In (AM2696, Thermo), 2 M Betaine, 20 mM Tris, 10 mM Ammonium sulfate, 6 mM MgSO4, 0.1% Triton-X, 1.3% DMSO, pH 8.8); each extension step has an incubation time of 1800 seconds.

Following 12 cycles of RNA extension, we have observed that for long templates (>800 nt or >900 nt), TGK is unable to completely synthesise the strand. We believe this to be due to a build up of torque in the DNA: RNA duplex that is covalently attached to the surface via the respective 5′ ends. In order to relieve the torque, we anneal an oligo over the 8-oxoG site on the grafted P7 primer and perform 2 cycles of cleavage (with Illumina's ‘Fast Linearisation Mix 2’ (FLM2) reagent or 200 U/ml Fpg, 100 μl/ml BSA and 1×NEBuffer 1) and extension (with TAM) at 37 C for 30 minutes and 55 C for 1 hour respectively.

Following DNA cleavage and final extensions, we denature the DNA: RNA duplex and wash away the DNA template using a mixture of 100 mM NaOH and 5 mM EDTA (or Illumina's FDR mix), before cleaning the flow cell with 2 ml of 6 M GuHCI, 10 mM Tris, pH 7.4, and 2 ml of 5×SSC, 0.1% Tween 20. With clusters of single stranded RNA present on the flow cell, 100 nM of R2_atto647N and P7′_surface_hyb is annealed to the P7 adaptor at the 3′ end of each molecule of RNA.

TABLE 3

| Oligo name | Sequence |
|---|---|
| R2_atto647N | /5ATTO647NN//iSpC3/GTGACTGGAGTTCAGACGTGTGCTCTTCCGAT |
| | C (SEQ ID NO: 18) |
| P7′_surface_hyb | GAACTCCAGTCACATCTCGTATGCCGTCTTCTGCTTG (SEQ ID NO: 19) |

### Ribosome Display on an Illumina Flow Cell

Ribosome display is performed using a custom PURExpress kit from New England Biolabs (NEB) that lacks release factors 1, 2 and 3, and also lacks T7 RNA polymerase. Specifically, we prepare a 200 ul master mix containing 80 ul of Solution A, 60 ul of Solution B, 4 ul of disulfide enhancers 1 and 2 (E6820S, NEB) (if required), 4 ul of Superase In (AM2696, Thermo), 10 ul of 10 mM Tris, pH 7.0, 4 M Trimethylamine N-Oxide and 10 ul of Millipore water (if required). We then inject 90 ul of the master mix into each lane of the flow cell using a custom designed low dead volume manifold, being careful to avoid the introduction of bubbles, before incubating the flow cell at 37° C. for 60 minutes on the HiSeq. Once the incubation period is complete, we cool the flow cell down to 20° C., before washing and stabilising the ribosomes with 1 ml per lane of ribosome display buffer (50 mM TrisAc (Tris(hydroxymethyl)aminomethane acetate), 150 mM NaCl, 50 mM MgAc (Magnesium acetate), 0.1% Tween 20, 1 U/ml of Superase In (AM2696, ThermoFisher), pH 7.5).

With the ribosomes stabilised by the display buffer, we next block the flow cell with the binding buffer (ribosome display buffer and 0.1% bovine serum albumin (BSA) (A9647, Sigma-Aldrich)) using 6×250 μl injections and a 10 minute wait between each injection. We then inject 100 nM of AF532 Streptavidin (S11224, ThermoFischer scientific) in binding buffer and incubate this at 20° C. for 30 minutes before washing the flow cell with 250 μl per lane of binding buffer and imaging. These images serve as a baseline for background fluorescence, non-specific binding, and any leftover fluorescence from sequencing.

### Sequencing of CDRs Via Internal Sequencing Primers

Following a successful deep screening display experiment, we setup a subsequent sequencing experiment using the same library for resolving the CDR sequences with internal sequencing primers. CDR sequencing experiments are performed in HCS with a custom recipe that initially sequences the N₂₈ UMI with Illumina's Read 1 sequencing primer for 28 cycles, followed by denaturation of the sequencing product with FDR at 65° C., annealing of an appropriate internal sequencing primer and sequencing enough cycles to cover the region of variability. All internal sequencing primers used in this work are ordered from IDT, HPLC purified and resuspended in IDTE at 100 μM.

Oligos used for internal sequencing of CDRs

| Oligo name | Seq cycles | Sequence |
|---|---|---|
| Kruse_Nb_CDR1_seq | 27 | GCCTGAGCTGCGCGGCGAGC (SEQ ID NO: 21) |
| Kruse_Nb_CDR2_seq | 42 | GCCAGGCGCCGGGCAAAGAACGC (SEQ ID NO: 22) |
| Kruse_Nb_CDR3_seq | 57 | CCGGAAGATACCGCGGTGTATTATTGCGCG |
| | | SEQ ID NO: 23) |
| IL7_scFv_VLCDR1_seq | 45 | GTCCCCAGGACAGACAGCCAGCATCACC (SEQ ID NO: 24) |
| IL7_scFv_VLCDR3_seq | 45 | CCGGGACCCAGGCTATGGATGAGGCTGAGTATTAC |
| | | (SEQ ID NO: 25) |
| Her2_G98A_VH3_seq | 63 | GCCCTCTGATTCTGCGGTATACTTCTGTGCTCGT (SEQ ID |
| | | NO: 26) |

Binding of Her2 to Anti-Her2 scFv Affinity Panel

The equilibrium binding assay is performed by preparing a dilution series of Her2-biotin (HE2-H822R, Acro biosystems) ranging from 0.03 nM to 100 nM in binding buffer, and a solution of 100 nM AF532 Streptavidin in binding buffer. Each step of the binding assay consists of 1) injecting 100 μl per lane of Her2-biotin, 2) incubating for 40 minutes, 3) washing with 100 μl per lane of binding buffer, 4) injecting 100 μl per lane of 100 nM AF532 streptavidin, 5) incubating for 10 minutes, 6) washing with 150 μl of binding buffer and 7) imaging the flow cell. This process is done step-wise from lowest concentration to highest.

Following the equilibrium binding experiment, pseudo kinetic off rates can be measured by injecting binding buffer into the flow cell at a fixed rate, and imaging at fixed time points. In this instance, the flow cell was imaged at 5, 10, 20, 60, 120 and 240 minute intervals.

### Image Processing

A single scan of a two-lane rapid run flow cell generates 8×2048×160,000 pixel 16-bit tif images in 4 colour channels, for a total of 32 images. The HiSeq 2500 uses a 532 nm and 660 nm laser with a set of emission filters (558-32 nm, 610-60 nm, 687-20 nm, and 740-60 nm) that path out to 4× time delayed integration (TDI) line scanning CCD detectors. We can detect signal from Alexa/Atto 647 on the ‘A’ and ‘C’ channels, and Alexa 532 on the ‘G’ and ‘T’ channels, with the highest signal to noise ratio observed on the ‘C’ and ‘T’ channels with these dyes. As such, we only perform analysis using the ‘C’ and ‘T’ colour channels.

Our image processing pipeline operates by breaking up each of the 2048×160,000 pixel images into 16 tiles which are processed independently in parallel. For a given tile image, we first perform a non-uniform illumination correction by applying a morphological opening with a disk shaped structuring element using a radius of 25 pixels before subtracting the morphological opening from the tile image. We then detect the centroids of any clusters present in the tile image using a peak local maxima function that operates initially by performing a morphological dilation of the tile image with a 3×3 pixel square kernel. The algorithm then moves through each pixel of the tile image and checks if the tile image pixel image is equal to the value of the same dilated pixel, and whether that pixel intensity is above a set threshold. If a given pixel meets these conditions, it is deemed to be a centroid, and is added to the centroid map. In this case, we are using a pixel intensity threshold of 400 or 600 (this value was manually tuned for our instrument). This method for cluster detection is simple, fast to compute and generally good enough for our needs.

Using the detected cluster coordinates on the ‘C’ and ‘T’ images, we align these against the known sequencing coordinates using a DFT (discrete Fourier transform) phase correlation function from the OpenCV package. As there are some slight variations in the repeatability of the microscope stage and optical distortion within the HiSeq, we perform a refined alignment by subdividing the tile image further into 128×128 pixel non-overlapping sub-images and saving the refined offsets to an offset map.

Using the refined offset map, we quantify the intensity of every known cluster from the sequencing data by extracting a 9×9 pixel sub-image centred on the offset corrected cluster coordinates. We then perform an element wise multiplication of the 9×9 pixel sub-image with a 9×9 pixel array constructed from a 2D Gaussian point spread function (PSF) with a sigma of 0.5. We use the following equation to describe our 2D Gaussian PSF:

$$1 exp ( - ( \frac{{( cx - x )}^{2}}{2 {σ}^{2}} + \frac{{( cy - y )}^{2}}{2 {σ}^{2}} ) )$$

$$or$$

$$PSF = 1 * {e}^{( - ( \frac{{( cx - x )}^{2}}{2 {σ}^{2}} + \frac{{( cy - y )}^{2}}{2 {σ}^{2}} ) )}$$

The sum of pixel values after the element wise multiplication is what we define to be the cluster intensity. The image processing pipeline reports cluster intensities for every sequenced cluster on the ‘C’ and ‘T’ channels from every scan of the flow cell and saves this to disk or inserts it into a database.

### Data Analysis

Barcode sequences and integrated cluster intensities are matched and grouped by unique barcode sequence through our custom data processing pipeline. This then performs outlier rejection using median absolute deviation and a cutoff of 2.0. General statistics are reported for each unique barcode: such as mean, median, standard deviation, standard error of the mean, minimum and maximum intensities. This is done for both the ‘C’ and ‘T’ channels, which allows for normalisation of the protein binding signal against the RNA probe signal.

In more detail, data analysis initially starts by grouping all cluster data by their common N28 UMI. If there are at least 12 replicates, where a cluster has not been rejected for falling outside of the imaging area, the UMI is retained. We next group the UMI and binding data with the UMI and CDR sequencing data, where there exist at least three CDR reads per UMI. Following the grouping, CDR reads are consensus error corrected (and the UMI is dropped if there is no consensus) before performing median absolute deviation outlier rejection and calculating mean, median, standard deviation, and standard error of the mean for each UMI on both the T (532 nm; protein) and C (660 nm: RNA) colour channels.

### Equilibrium Binding and Dissociation Rate Fitting

With the binding data grouped by unique barcode and outliers removed, we plotted the median binding data for each of the anti-Her2 scFv library members, and fitted equilibrium binding curve using the following equation and a least squares fit with a trust region reflective algorithm as the solver.

$$y = \frac{F max}{\frac{Kd}{x} + 1} + F min$$

Where Fmax is the maximum intensity of a given clone, Fmin is the minimum intensity of a given clone, Kd is the value to be fit, and x is the concentration of antigen for a given median intensity (v).

To fit the kinetic off rates, the following equation was fit to the data using least squares and a trust region reflective solver.

$$R = {R}_{1} {e}^{- {k}_{d 1} ( t - t 0 )} + ( {R}_{0} - {R}_{1} ) {e}^{- {k}_{d 2} ( t - t 0 )}$$

Where t is time in seconds, R₁ is the initial response level for component 1, k_{di} is the dissociation rate constant for component i, R₀ is the total response level at the start of dissociation, t0 is the start time for the dissociation.

The equilibrium binding and dissociation rate fitting may alternatively be described as follows:

Flow cell based equilibrium binding curves are fit using the following equation to the mean integrated intensities of a given UMI via least squares, as implemented in the curve_fit( ) function from the python Package SciPy.

$$R = \frac{F max}{1 + ( \frac{KD}{x} )} + F min$$

Where Fmax is the maximum intensity observed, Fmin is the minimum intensity observed, KD is the equilibrium binding constant that we wish to fit, and x is concentration of a given measurement.

Flow cell based kinetic dissociation curves are fit using the following biphasic dissociation equation via least squares, as implemented in the curve_fit( ) function from the python Package SciPy.

$$R = R 1 * {e}^{( - kd 1 * ( t - t 0 ) )} + ( R 0 - R 1 ) * {e}^{( - kd 2 * ( t - t 0 ) )}$$

Where R0 is the intensity observed at the start of dissociation, R1 is a floating parameter for the initial intensity for component 1, t is time in seconds, 10 is the start time for the dissociation and kdi is the dissociation rate constant for component i.

### Nanobody Yeast Surface Display Selections

The nanobody yeast display library was acquired from the Kruse laboratory as a frozen stock of >2.5×10⁹ cells (EF0014-FP, Kerafast). The library aliquots were initially thawed at 30° C., before being recovered in 1 L of ‘Yglc4.5-Trp’ (3.8 g/L-Trp yeast dropout media supplement (Y1876, Merck), 6.7 g/L yeast nitrogen base (Y0626, Merck), 10 mL/L Pen-Strep (P4333, Merck)), shaking at 230 RPM, 30° C., overnight. The recovered culture was then expanded to 3 L of media and allowed to grow to a stationary phase (OD₆₀₀ of 20) over 48 hours. The culture was centrifuged at 3,500×g for 5 minutes and resuspended in fresh Yglc4.5-Trp supplemented with 10% DMSO, such that the final density is 10¹⁰ cells per mL before making 2 mL aliquots and freezing at −80° C.

To prepare the naïve library for the first round of selection, one aliquot was thawed at 30° C. and used to inoculate 1 L of Yglc4.5-Trp supplemented with 2% galactose. The culture was then grown for 72 hours at 24° C. Expression was confirmed by flow cytometry with a FITC labelled anti-HA antibody (GG8-1F3.3.1, Miltenyi Biotech) prior to the first round of selection. Cells representing over ten-fold the library diversity were initially deselected against streptavidin microbeads (Miltenyi Biotech) for one hour at 4° C. in PBS-T-BSA (0.1% Tween-20, 0.1% BSA) before being separated from the beads on a Miltenyi MACS magnet. Deselected cells were then incubated in the presence of 500 nM HEL-biotin (GTX82960-pro, GeneTex) for one hour at 4° C. Streptavidin beads were added and incubated further for 15 minutes prior to selection and washing on a Miltenyi MACS magnet. Beads and the bound cells were eluted, pelleted, and resuspended in 1 L of Yglc4.5-Trp supplemented with 2% galactose prior to growth for 72 hours at 24° C. Round 2 was conducted similar to round 1, with the absence of a deselection cells and reduction to 300 nM HEL-biotin before adding streptavidin microbeads, panning on a MACS column, washing and recovering the cells.

Following round 2, the recovered cells were split in half by volume to conduct a round 3 via MACS (magnetic activated cell sorting) and FACS (fluorescence activated cell sorting) with the respective splits. Round 3 MACS was conducted as per round 2 with a further reduction to 200 nM HEL-biotin, followed by recovery, harvesting of cells by centrifugation and miniprep of the plasmid DNA (D2004, Zymo Research). Prior to harvesting cells, 100 μL of cells was serially diluted and plated on YPD agar plates to enable picking of 96 colonies for colony PCR and Sanger sequencing. Round 3 FACS was conducted by incubating cells with 200 nM HEL-biotin for one hour at 4° C., pelleted and resuspended in fresh PBS-T-BSA and combined with 100 μg of Neutravidin-PE (A2660, ThermoFisher Scientific) and a 1:1000 dilution of the anti-HA-FITC antibody for 15 minutes before being sorted on a Synergy 3 cell sorter (Sony Biotechnology) and gating for dual labelled (FITC/PE) events, yielding 50,135 cells. Sorted cells were recovered and miniprepped as per round 3 MACS.

### Nanobody Library Preparation and Deep Screening

Minipreps for round 3 MACS and FACS were PCR amplified (Q5 polymerase: M0492, NEB) for 20 cycles using primers that anneal with the N terminal framework region, C-terminal HA tag and introduce a 20 nucleotide overhangs at the 5′ end of each primer that contain homology with the 5′ flow cell adapter (RBS+ATG; KF_olap.fwd) and the 3′ flow cell adapter (TolAk linker; KF_olap.rev).

| Oligo name | Sequence |
|---|---|
| KF_olap.fwd | ATTAAGGAGGTATATACATGCAGGTGCAGCTGCAG |
| | GAAAG (SEQ ID NO: 27) |
| KF_olap.rev | TGACCCAAATTCGGCACCACTAGCCATATAAGCGT |
| | AATCTGGAACATCGTATGGG (SEQ ID NO: 28) |

The nanobody library, now containing homology with the adapters was run on a 1% agarose gel and a band of approximately 449 bp was gel extracted (approximate, as the library contains variable sized CDR loops), purified and quantified by nanodrop. The library is subsequently assembled into the deep screening display construct via Gibson assembly using 0.2 pmol of the 5′ adaptor, the nanobody library fragment and 3′ adaptor and the HiFi DNA assembly master mix (E2621, NEB) and incubated at 50° C. for 30 minutes. The library is then bottlenecked by taking 300 amol of material from the Gibson assembly reaction (assuming 100% efficiency) and PCR amplifying for 25 cycles with Q5 polymerase and the outnest P5 and P7 primers.

| Oligo name | Sequence |
|---|---|
| P5_PCR.fwd | AATGATACGGCGACCACCGA |
| | (SEQ ID NO: 29) |
| P7_PCR.rev | CAAGCAGAAGACGGCATACGAGAT (SEQ ID |
| | NO: 30) |

The PCR product was run on a 1% agarose gel and a roughly 800 bp band was gel extracted, purified and quantified initially by nanodrop and subsequently by qPCR (NEBNext library quant kit, E7630, NEB).

The quantified library, now ready for deep screening, was diluted to 2 nM before being denatured (10 μL of library is mixed with 10 μL of 100 mM NaOH and incubated at RT for 5 minutes) and rapidly diluted to 20 pM in HT1 buffer provided by the rapid PE flow cell clustering kit (PE-402-4002, Illumina). We then dilute the library to a concentration of 6 pM before loading into the template slot on the HiSeq 2500 and setting up a deep screening experiment as previously described.

Following acquisition of the baseline flow cell images, we performed an equilibrium binding assay at successive and increasing concentrations of HEL-biotin. Specifically, each condition involves an injection of 120 μL of HEL-biotin (GTX82960-pro, GeneTex) that had been pre-complexed with AF532-Streptavidin (S11224, ThermoFisher) at a 1:1 ratio in display buffer at 20° C., an incubation of 45 minutes at 20° C., a 200 μL wash of display buffer, followed by complete imaging of the flow cell. This was performed for 1 nM, 10 nM, 100 nM and 300 nM HEL with 1:1 amounts of AF532-Streptavidin. Following the highest concentration of HEL, we proceeded to collect measurements for a kinetic dissociation rate. This was accomplished by pumping display buffer over the flow cell and imaging at 5, 10, 15, 20, 30, 60 and 120 minutes. Raw images were then processed as described above.

### Nanobody Expression and Periplasmic Extraction

Nanobody hits were computationally composed assuming no mutations were present outside of the sequenced CDR regions, which contains 3 nucleotides before and after the actual variability. Composed hits were then codon optimised and ordered as a gBlock from IDT before being cloned via FX cloning into the E. coli periplasmic expression vector pSBinit, a gift from Markus Seeger (Addgene plasmid #110100: http://n2t.net/addgene: 110100: RRID: Addgene_110100). Single colonies were picked, and correct clones validated by Sanger sequencing. Following validation, single colonies were grown overnight in 5 mL of TB+25 μg/mL chloramphenicol at 37° C. before being sub-cultured at 1:100 into 5 mL of TB (w/chloramphenicol). Cultures were grown at 37° C. and induced roughly at an OD₆₀₀ of 0.6-0.9 with 0.05% w/v L-arabinose. Cultures were grown for another 3.5 hours before being harvested by centrifugation at 2,500×g for 20 minutes at 4° C. and supernatant discarded. Pellets were resuspended (1/20 of the original culture volume) in 250 μL of TES buffer (50 mM Tris-HCl, pH 7.2, 0.1 mM EDTA, 20% sucrose) and incubated on ice for 60 minutes to perform a periplasmic extraction. The supernatant was then collected by centrifugation at 20,000×g for 30 minutes at 4° C. and protein yield quantified by SDS page. All clones were normalised to a concentration of 500 nM in SuperBlock PBS (37515, ThermoFisher Scientific) prior to BLI kinetics measurements.

### Nanobody Kinetics Measurements

Periplasm extracted nanobodies that had been normalised to 500 nM in SuperBlock PBS were further diluted to 50 nM. BioLayer Interferometry (BLI) kinetics were performed on an Octet Red384 (Sartorius) with reference subtraction performed for each nanobody clone using a non-loaded streptavidin tip (18-5136, Sartorius). Kinetics were measured using the following steps: 1) Sensor check for 30 seconds, 2) Loading of HEL-biotin at 25 μg/mL for 400 seconds, 3) Baseline measurement for 240 seconds 4) Association kinetics at 50 nM of each nanobody for either 400 or 500 seconds, 5) Dissociation kinetics for 600 seconds. In all stages, SuperBlock PBS was used as the buffer.

### Biolayer Interferometry Data Fitting

BLI kinetics data was collected on an Octet Red384 instrument as described in the previous and subsequent kinetics measurements sections. In all cases, streptavidin tips (18-5136, Sartorius) were loaded with biotinylated target antigen and washed to a baseline signal before binding at a fixed concentration of each VHH or Fab clone. Following on rate kinetics collection, tips were dipped in fresh buffer to measure off rate kinetics. Measurement data for each clone was referenced against streptavidin only tips to remove non-specific binding to streptavidin.

A 1:1 model was fit to all data via least squares using a custom python script.

Association rates were fit to the following equation:

$$Rassoc = R max * ( \frac{1}{1 + \frac{Kd}{Ka * C}} ) * ( 1 - {e}^{( - Ka * C * Kd ) * t} )$$

Where Rmax is the peak response, Kd is the dissociation rate to be estimated, Ka is the association rate to be determined, C is the concentration of the Fab in molar and t is time in seconds.

Dissociation rates were fit to the following equation:

$$Rdissoc = Y 0 * {e}^{- Kd ( t - t 0 )}$$

Where Y0 is equal to Rassoc at the end of the association phase, Kd is the dissociation rate to be determined, t is the current time in seconds and t0 is the time at the start of the dissociation phase.

K_{D} values are calculated as:

$${K}_{D} = \frac{Kd}{Ka}$$

### IL-7 Library Preparation and Deep Screening

The unselected IL-7 VK light chain CDR L1 and L3 scFv library was prepared and provided to us by AstraZeneca in the pCANTAB6 plasmid. The scFv library was extracted by 20 cycles PCR using Q5 polymerase and primers that provide 25 nucleotides of homology with the 5′ and 3′ display adapters. The PCR product was run on a 1% agarose gel, and a roughly 778 bp band was gel extracted and purified. Similar to the nanobody library assembly, 0.2 pmol of the 5′ adaptor, the scFv library fragment and 3′ adaptor and the HiFi DNA assembly master mix (E2621, NEB) is combined and incubated at 50° C. for 30 minutes. The library is then bottlenecked by taking 500 amol of material from the Gibson assembly reaction (assuming 100% efficiency) and PCR amplifying for 25 cycles with Q5 polymerase and the outnest P5 and P7 primers. The PCR product was run on a 1% agarose gel and a 1.2 kb band was gel extracted, purified and quantified initially by nanodrop and subsequently by qPCR (NEBNext library quant kit, E7630, NEB).

The quantified library, now ready for deep screening, was diluted to 2 nM before being denatured (10 μL of library is mixed with 10 μL of 100 mM NaOH and incubated at RT for 5 minutes) and rapidly diluted to 20 pM in HT1 buffer provided by the rapid PE flow cell clustering kit (PE-402-4002, Illumina). We then dilute the library to a concentration of 6 pM before loading into the template slot on the HiSeq 2500 and setting up a deep screening experiment as described above.

Following acquisition of the baseline flow cell images, we performed an equilibrium binding assay at successive and increasing concentrations of hu-IL7-biotin pre-complexed with AF532-Streptavidin (S11224, ThermoFisher) in a 1:1 ratio (100 pM, 333 PM and 1 nM). In this experiment, we observed substantial aggregation of hu-IL-7 on the flow cell surface that prohibited imaging past a concentration of 1 nM hu-IL-7: as such, no kinetic dissociation measurement was collected. Images were processed and CDR sequences resolved as described above, which we used to identify putative hits.

### Anti-IL-7 and Anti-Her2 Fab Expression and Purification

The top 19 putative anti-IL7 hits (and IL70001) and all 26 anti-Her2 hits (including G98A, and ML3-9) were converted from scFv to Fab format, with the heavy and light chain variables being synthesised separately and cloned into mammalian expression vectors pEU10.1 and pEU4.4 respectively. Vectors were transiently transfected into CHO (Chinese Hamster Ovary) cells using PEI and a proprietary medium. Expressed Fabs were purified by loading the cleared culture supernatant onto a CaptureSelect™ CHI-XL column (Life Technologies, ThermoFisher, Netherlands), run in DPBS and eluted with 25 mM Acetate pH 3.6 and buffer exchanged into DPBS pH 7.4 using PD-10 desalting columns (Cytiva). The concentration was determined spectrophotometrically using an extinction coefficient based on the amino acid sequence. The protein purity was verified by SDS-PAGE and the verification of correct MW was achieved by LC-MS analysis. Analytical HP-SEC was performed post purification by loading 70 μl of each protein onto a TSKgel G3000SWXL: 5 μm, 7.8 mm×300 mm column using a flow rate of 1 ml/min and 0.1 M Sodium Phosphate Dibasic anhydrous+0.1 M Sodium Sulphate, pH 6.8 as the running buffer. A gel filtration standard (BIORAD, Cat no: 151-1901) was also run for comparative purposes.

### IL-7 Kinetics Measurements

Kinetics of binding for the top 19 hits and IL70001 was measured using Octet BLI and streptavidin coated tips (18-5136, Sartorius). In all cases the buffer used was DPBS (14190-169, Gibco)+0.1% BSA+0.02% Tween-20. Purified Fabs were diluted to a final concentration of 50 nM. Kinetics were measured using the following steps: 1) Sensor check for 60 seconds, 2) Loading of hu-IL7-biotin at 5 μg/mL for 30 seconds, 3) Baseline measurement for 60 seconds 4) Association kinetics at 50 nM of each Fab for 300 seconds, 5) Dissociation kinetics for 600 seconds.

### TF-1 STAT5 IL7R Alpha+Gamma Cell-Based Reporter Assay

Two vials containing 1 ml of 10⁷/ml TF-1 STAT5 IL7 alpha+gamma luciferase cG3 cells were removed from liquid nitrogen, defrosted, and transferred into 1×50 ml Falcon tubes (2 vials per tube) containing 40 mL of complete medium and centrifuged for 5 minutes at 1,200 rpm. The supernatant was aspirated, and cell pellets resuspended in 40 ml RPMI (11875093 ThermoFisher)+10% FBS+1% sodium pyruvate before centrifugation for another 5 minutes at 1,200 rpm before aspirating the supernatant as before. Cells were finally resuspended in 40 ml RPMI+10% FBS+1% sodium pyruvate, placed in a T175 flask and incubated for 24 hours at 37° C. in an atmosphere of 5% CO₂.

Hu-IL7 (CHO expressed) was made up to 0.12 nM in RPMI+10% FCS+sodium pyruvate, which was then diluted 1:100 to a final volume of 20 mL for addition to a 384 well plate. Purified Fabs were added undiluted to a 384 well plate, and an 11 point three-fold duplicate serial dilution was performed using a Bravo liquid handling platform into complete RPMI. Cells were removed following the 24-hour incubation and pelleted by centrifugation at 1,200 rpm for 5 minutes and resuspended in 10 mL of RPMI+10% FCS+1% sodium pyruvate. Cells were counted and diluted in complete RPMI to give a concentration of 10,000 cells/20 μL. Cells (20 μL) were then added to 3×384 well clear assay plates. 10 μL of the titrated Fabs were added to the cells, followed by 10 μL of 120 pM Hu-IL7. The plates were then placed in a tissue culture incubator for 6 hours at 37° C. in an atmosphere of 5% CO₂. 100 mL of Steady-Glo reagent (E2520, Promega) was defrosted prior to use and 40 μL was added to each well of the 384 well plates. The plates were sealed and incubated for 10 minutes in a plate shaker prior to measurement. Luminescence readings were measured using an EnVision plate reader with a 1 second pulse time. Each Fab was measured in duplicate.

Data was exported and processed using a custom python script, and mean data fitted using least squares to a log inhibitor response curve defined by the following equation.

$$Y = Bottom + \frac{( Top - Bottom )}{( 1 + {10}^{( ( LogIC 50 - X ) * HillSlope )} )}$$

Where Y is the response, Bottom is the response at the minimum of the sigmoid curve, Top is the response at the maximum of the sigmoid curve, LogIC50 is the log concentration of the inhibitor that gives a response half-way between the Top and the Bottom and HillSlope describes the steepness of the curve.

### Deep Screening of the Anti-Her2 Affinity Panel

The anti-Her2 scFv affinity panel plus Herceptin protein sequences were backtranslated, codon optimised and composed into the deep screening display construct with a known 28 nucleotide UMI. DNA constructs were ordered a gBlocks from IDT and clustered on a rapid PE flow cell at 1% per construct, with the remaining clusters on the flow cell comprising PhiX control (FC-110-3001, Illumina). The flow cell was sequenced for 28 cycles and deep screening display conducted as described above.

The nucleic acid sequences of the anti-Her2 scFv clones are shown in Table 2.

Following successful display, we performed an equilibrium binding assay using biotinylated human Her2 (HE2-H822R-25 ug, Acro Biosystems) and AF532-Streptavidin (S11224, ThermoFisher). In this instance, a binding assay cycle was conducted by injecting 120 μL of Her2-biotin, incubating for 45 minutes at 20° C., washing with 200 μL of display buffer, injecting 120 μL of 100 nM AF532-Streptavidin, incubating for 10 minutes at 20° C. before washing with 200 μL of display buffer and imaging. The equilibrium binding assay was performed at 100 pM, 333 pM, 1 nM, 3.33 nM, 10 nM, 33.3 nM and 100 nM Her2-biotin before initiating a kinetic dissociation assay. The dissociation assay was performed by pumping wash buffer over the flow cell and imaging at 5 minutes, 10 min, 20 min, 60 min, 240 min and 420 min. Data collected from this experiment was processed as described above, and aggregate statistics calculated through grouping by the known UMIs.

Anti-Her2 scFv Affinity Maturation Library Preparation and Deep Screening

We built a CDR VH3 affinity maturation library with G98A as the parental starting clone. This was accomplished by TOPO cloning (450245, ThermoFisher) the G98A gBlock from the previous section into TOP10 chemically competent cells (C404010, ThermoFisher), picking 6 colonies, growing these overnight in 5 mL TB+50 μg/mL kanamycin and miniprepping 2 mL of culture. Plasmids were sent for Sanger sequencing using M13 forward and reverse primers: one of the correct colonies were taken forward for subsequent processing.

As we wanted to build a VH3 affinity maturation library, we first needed to extract the regions upstream and downstream of VH3. We did this by PCR amplification of the plasmid DNA as two reactions for 25 cycles using Q5 polymerase with primer set 1 (G98A_olap.fwd and G98A_5p_VH3.rev) and primer set 2 (G98A_3p_VH3.fwd and G98A_olap.rev). Both PCRs were subsequently treated with DpnI (R0176L, NEB) for 1 hour at 37° C. before being purified with a PCR clean up kit (T1030S, NEB). This process yielded the upstream and downstream fragments of the G98A clone with homology to the deep screening display construct while removing contaminating wild-type plasmid DNA.

| Oligo name | Sequence |
|---|---|
| G98A_olap.fwd | ATTAAGGAGGTATATACATGCAGGTACAGCTTGTGCAG (SEQ ID NO: 31) |
| G98A_5p_VH3.rev | ACGAGCACAGAAGTATACCGCA (SEQ ID NO: 32) |
| G98A_3p_VH3.fwd | TGGGGACAAGGGACCCTTGTCAC (SEQ ID NO: 33) |
| G98A_olap.rev | TGACCCAAATTCGGCACCACTAGCCATATACCCAAGCACAGTAAGCTTCGTCC |
| | (SEQ ID NO: 34) |
| G98A_VH3_NNS_1 | CGGTATACTTCTGTGCTCGTNNSNNSNNSNNSTATTGTTCCAGTAGCAATTGCG |
| | CAAAGTGGCCTGAGTATTTCCAACATTGGGGACAAGGGACCCTTGT |
| | (SEQ ID NO: 35) |
| G98A_VH3_NNS_2 | CGGTATACTTCTGTGCTCGTCATGACGTCNNSNNSNNSNNSAGTAGCAAT |
| | TGCGCAAAGTGGCCTGAGTATTTCCAACATTGGGGACAAGGGACCCTTGT |
| | (SEQ ID NO: 36) |
| G98A_VH3_NNS_3 | CGGTATACTTCTGTGCTCGTCATGACGTCGCCTATTGINNSNNSNNSNNS |
| | TGCGCAAAGTGGCCTGAGTATTTCCAACATTGGGGACAAGGGACCCTTGT |
| | (SEQ ID NO: 37) |
| G98A_VH3_NNS_4 | CGGTATACTTCTGTGCTCGTCATGACGTCGCCTATTGTTCCAGTAGCNNS |
| | NNSNNSNNSTGGCCTGAGTATTTCCAACATTGGGGACAAGGGACCCTTGT |
| | (SEQ ID NO: 38) |
| G98A_VH3_NNS_5 | CGGTATACTTCTGTGCTCGTCATGACGTCGCCTATTGTTCCAGTAGCAATT |
| | GCGCANNSNNSNNSNNSTATTTCCAACATTGGGGACAAGGGACCCTTGT |
| | (SEQ ID NO: 39) |
| G98A_VH3_NNS_6 | CGGTATACTTCTGTGCTCGTCATGACGTCGCCTATTGTTCCAGTAGCAATTG |
| | CGCAAAGTGGCCTNNSNNSNNSNNSCATTGGGGACAAGGGACCCTTGT |
| | (SEQ ID NO: 40) |

We next assembled the Her2 affinity maturation library by 20 cycles of PCR using Q5 polymerase, the upstream and downstream fragments of G98A, an equimolar amount of VH3 NNS oligos that produce a scanning window of 4 NNS codons across the CDR VH3, and the G98A olap forward and reverse primers. This product is then column purified using a PCR clean up kit (T1030S, NEB). We next append the deep screening 5′ and 3′ adapters using Gibson assembly with 0.2 pmol of each fragment and NEB HiFi assembly master mix (E2621, NEB) at 50° C. for 60 minutes. The library is then bottlenecked by taking 300 amol of material from the Gibson assembly reaction (assuming 100% efficiency) and PCR amplifying for 25 cycles with Q5 polymerase and the outnest P5 and P7 primers. The PCR product was run on a 1% agarose gel and a 1.2 kb band was gel extracted, purified, and quantified initially by nanodrop and subsequently by qPCR (NEBNext library quant kit, E7630, NEB).

The quantified library, now ready for deep screening, was diluted to 2 nM before being denatured (10 μL of library is mixed with 10 μL of 100 mM NaOH and incubated at RT for 5 minutes) and rapidly diluted to 20 pM in HT1 buffer provided by the rapid PE flow cell clustering kit (PE-402-4002, Illumina). We then dilute the library to a concentration of 6 pM before loading into the template slot on the HiSeq 2500 and setting up a deep screening experiment as described above.

Following acquisition of the baseline flow cell images, we performed an equilibrium binding assay at successive and increasing concentrations of human Her2-biotin (HE2-H822R-25 ug, Acro Biosystems) pre-complexed with AF532-Streptavidin (S11224, ThermoFisher) in a 1:1 ratio (100 pM. 333 pM, 1 nM, 3.33 nM, 10 nM, 33.3 nM and 100 nM). In this instance, a binding assay cycle was conducted by injecting 120 μL of the Her2-biotin: AF532-streptavidin pre-complex, incubating for 45 minutes at 20° C., washing with 200 μL of display buffer before imaging the flow cell. Following the highest 100 nM condition, a kinetic dissociation assay was conducted by pumping display buffer over the flow cell and imaging at 5 minutes, 10 mins, 20 mins, 60 mins, 120 mins and 240 mins. Images were then processed, and CDR sequences resolved through internal primer sequencing as described above, which we used to assemble a CDR: binding dataset termed ‘HER2affmat’.

ML vs. Random Library Preparation and Deep Screening

We devised a selection scheme where for each seed sequence a random mutation set was compiled from all single mutants and up to 1000 mutants from edit distances 2-5 yielding pool of 13,121 mutations (‘random/mut’). We next assembled a pool of sequences with exclusively machine learning generated mutations by removing all sequences with a high-hit score<0.9 and randomly selecting up to 1000 mutants from edit distances 2-5 as well as rejecting those that were already selected in the ‘random/mut set. This assembled a pool of 11,916 mutations (‘ml/mut’). Sequences were combined into an oligo pool of 25,042 CDR VH3 sequences and ordered from Twist Bioscience. The “Her2 ML vs. random” library was assembled for deep screening similar to the “HER2affmat” library, where 20 cycles of PCR using Q5 polymerase, the upstream and downstream fragments of G98A, were combined with the oligo pool, and the G98A olap forward and reverse primers. This product was then column purified using a PCR clean up kit (T1030S, NEB). We next append the deep screening 5′ and 3′ adapters using Gibson assembly with 0.2 pmol of each fragment and NEB HiFi assembly master mix (E2621, NEB) at 50° C. for 60 minutes. The library is then bottlenecked by taking 300 amol of material from the Gibson assembly reaction (assuming 100% efficiency) and PCR amplifying for 25 cycles with Q5 polymerase and the outnest P5 and P7 primers. The PCR product was run on a 1% agarose gel and a 1.2 kb band was gel extracted, purified, and quantified initially by nanodrop and subsequently by qPCR (NEBNext library quant kit, E7630, NEB).

The quantified library, now ready for deep screening, was diluted to 2 nM before being denatured (10 μL of library is mixed with 10 μL of 100 mM NaOH and incubated at RT for 5 minutes) and rapidly diluted to 20 pM in HT1 buffer provided by the rapid PE flow cell clustering kit (PE-402-4002, Illumina). We then dilute the library to a concentration of 6 pM before loading into the template slot on the HiSeq 2500 and setting up a deep screening experiment as described above. Following acquisition of the baseline flow cell images, we performed an equilibrium binding assay at successive and increasing concentrations of human Her2-biotin (HE2-H822R-25 ug, Acro Biosystems) pre-complexed with AF532-Streptavidin (S11224, ThermoFisher) in a 1:1 ratio (100 pM, 333 pM, 1 nM, 3.33 nM, 10 nM, 33.3 nM and 100 nM). In this instance, a binding assay cycle was conducted by injecting 120 μL of the Her2-biotin: AF532-streptavidin pre-complex, incubating for 45 minutes at 20° C., washing with 200 μL of display buffer before imaging the flow cell. Following the highest 100 nM condition, a kinetic dissociation assay was conducted by pumping display buffer over the flow cell and imaging at 5 minutes, 10 mins, 20 mins, 60 mins, 120 mins and 240 mins. Images were then processed, and CDR sequences resolved through internal primer sequencing as described above, which we used to assemble a CDR: binding dataset termed ‘Her2 ML vs. random’.

### Anti-Her2 Hit Kinetics Measurements

Kinetics of binding for all anti-Her2 Fabs was measured using Octet BLI and streptavidin coated tips (18-5136, Sartorius). In all cases the buffer used was DPBS (14190-169, Gibco)+0.1% BSA+0.02% Tween-20. Purified Fabs were diluted to a final concentration of 20 nM. Kinetics were measured using the following steps: 1) Sensor check for 60 seconds, 2) Loading of human Her2-biotin (HE2-H822R-25 ug, Acro Biosystems) at 5 μg/mL for 30 seconds, 3) Baseline measurement for 60 seconds 4) Association kinetics at 20 nM of each Fab for 300 seconds, 5) Dissociation kinetics for 600 seconds in buffer.

TABLE S2
ML model train:test confusion matrix.

| | Count | TP | FP | TN | FN |
|---|---|---|---|---|---|
| Train | | | | | |
| Non-hit | 209,414 | 206,564 | 277 | 988 | 2,850 |
| Low-hit | 1,168 | 839 | 2,882 | 206,629 | 329 |
| High-hit | 97 | 53 | 64 | 210,518 | 44 |
| Test | | | | | |
| Non-hit | 23,279 | 22,984 | 31 | 99 | 295 |
| Low-hit | 116 | 82 | 301 | 22,992 | 34 |
| High-hit | 14 | 6 | 5 | 23,390 | 8 |

TABLE S3
ML model train:test precision, recall and F1-score.

| | Precision* | Recall** | F1-score*** |
|---|---|---|---|
| Train | | | |
| Non-hit | 0.999 | 0.986 | 0.992 |
| Low-hit | 0.225 | 0.718 | 0.343 |
| High-hit | 0.453 | 0.546 | 0.495 |
| Test | | | |
| Non-hit | 0.999 | 0.987 | 0.993 |
| Low-hit | 0.214 | 0.707 | 0.329 |
| High-hit | 0.545 | 0.429 | 0.480 |

*Precision is defined as: TP/(TP + FP)
**Recall is defined as: TP/(TP + FN)
***F1-score is defined as the harmonic mean of precision and recall.

TABLE S4
ML vs. Random selected clones; hit performance.

| | | | Random | | | | ML fold |
|---|---|---|---|---|---|---|---|
| Edit | Random | Random | hit rate | ML | ML | ML hit | improve- |
| distance | total | hits | (%) | total | hits | rate (%) | ment |
| 1 | 1,140 | 154 | 13.51 | 220 | 78 | 35.45 | 2.62 |
| 2 | 2,981 | 96 | 3.22 | 2,932 | 746 | 25.44 | 7.90 |
| 3 | 3,000 | 40 | 1.33 | 2,984 | 447 | 14.98 | 11.26 |
| 4 | 3,000 | 8 | 0.27 | 3,000 | 264 | 8.80 | 32.59 |
| 5 | 3,000 | 5 | 0.17 | 3,000 | 119 | 3.97 | 23.35 |
| Total | 13,121 | 303 | 2.31 | 12,136 | 1,654 | 13.62 | 5.90 |
| | | | Total* | 11,916 | 1,576 | 13.23 | 5.73 |

*This total excludes the single point mutations from the ML set.

The invention may be described by reference to the following non-limiting clauses, which define particular aspects and embodiments of the invention.
1. A method of displaying a non-DNA nucleic acid molecule on a substrate, comprising:
   i) providing a first nucleic acid immobilised on a substrate, and wherein the first nucleic acid is oriented such that the 5′ end is proximal and the 3′ end is distal to the point of immobilisation;
   ii) generating a second nucleic acid that is complementary to the first nucleic acid, wherein the generation of the second nucleic acid comprises:
      a) contacting the first nucleic acid with a nucleic acid polymerase under conditions suitable for polymerisation, wherein
      the primer for polymerisation is a DNA primer immobilised on the substrate such a bridge is formed during polymerisation,
      the product of the polymerisation is a chain of non-DNA nucleotides that is immobilised on the substrate via the primer, and
      the nucleic acid polymerase is a polymerase capable of acting upon a DNA primer to synthesise a non-DNA nucleic acid molecule that is complementary to a single-stranded nucleic acid template; and
   iii) removing the first nucleic acid to result in display of the second nucleic acid on the substrate.
2. The method of clause 1, wherein the second nucleic acid is an RNA molecule.
3. The method of clause 2, wherein the nucleic acid polymerase comprises an amino acid sequence having at least 36% identity to the amino acid sequence of SEQ ID NO: 1, wherein said amino acid sequence comprises a Y409 and an E664 mutation relative to the amino acid sequence of SEQ ID NO: 1: optionally wherein the Y409 mutation is Y409G and the E664 mutation is E664K; and optionally wherein the amino acid sequence of the nucleic acid polymerase comprises SEQ ID NO: 3.
4. The method of clause 1, wherein the second nucleic acid is an XNA molecule.
5. The method of clause 4, wherein the XNA molecule comprises an arabinonucleotide, an arabinonucleic acid (ANA) nucleotide, a 2′-Fluoro-arabinonucleic acid (FANA) nucleotide, a 2′-O-methyl ribonucleic acid (2′OMe) nucleotide, a 2′-O-methoxyethyl (MOE) nucleic acid nucleotide, a phosphorothioate 2′-O-methoxyethyl (PS-MOE) nucleotide, a phosphorodiamidate morpholino nucleotide, a locked nucleic acid (LNA) nucleotide, a P-alkyl phosphonate nucleic acid (phNA) nucleotide, a threose nucleic acid (TNA) nucleotide, a hexitol nucleic acid (HNA) nucleotide, a 2′ hydroxy-hexitol (AtNA) nucleotide, a cyclohexene nucleic acid (CeNA) nucleotide, or a 3′ deoxi-DNA (2′-5′) nucleotide.
6. The method of one of clauses 1 to 5, wherein the nucleic acid polymerase comprises an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide.
7. The method of clause 6, wherein the amino acid sequence of the nucleic acid polymerase comprises one or more, or all, of the following mutations: V93Q, D141A, E143A, and A485L.
8. The method of any one of clauses 1 to 7, wherein the polymerase is TGK, TGLLK, 2M, Bst, RT521, 6G12, 6G12521, C7, PGLVV, PGLVVWA, D4K, or a variant thereof.
9 The method of any one of clauses 1 to 8, wherein after step ii) a), the method comprises cleaving the first nucleic acid and linearizing the bridge.
10. The method of clause 9, further comprising re-contacting the linearized product with the nucleic acid polymerase under conditions suitable for polymerisation.
11. A method of displaying a non-DNA nucleic acid molecule on a substrate, comprising:
   i) providing a first nucleic acid immobilised on a substrate, and wherein the first nucleic acid is oriented such that the 5′ end is proximal and the 3′ end is distal to the point of immobilisation;
   ii) generating a second nucleic acid that is complementary to the first nucleic acid, wherein the generation of the second nucleic acid comprises:
      a) contacting the first nucleic acid with a nucleic acid polymerase under conditions suitable for polymerisation, wherein
      the primer for polymerisation is immobilised on the substrate such a bridge is formed during polymerisation, and
      the product of the polymerisation is a chain of non-DNA nucleotides that is immobilised on the substrate via the primer:
      b) cleaving the first nucleic acid and linearizing the bridge; and
      c) contacting the linearized product of step b) with a polymerase under conditions suitable for polymerisation; and
   iii) removing the first nucleic acid to result in display of the second nucleic acid on the substrate.
12. The method of any one of clauses 1 to 11, wherein step ii) a) comprises at least 5, 10, 12, 15, 20, or 25 cycles of bridged polymerisation.
13. The method of any one of clauses 1 to 12, wherein the first nucleic acid is removed in step iii) by contacting the first nucleic acid with a denaturation reagent, wherein the denaturation reagent is a buffer comprising 100 mM NaOH and 5 mM EDTA.
14. The method of any one of clauses 1 to 13, wherein the second nucleic acid is an RNA molecule and encodes a polypeptide, and further comprising:
   iv) contacting the second nucleic acid with a ribosome under conditions suitable for translation of the encoded polypeptide: optionally wherein the conditions of step iv) comprise trimethylamine N-oxide (TMAO).
15. The method of clause 14, wherein encoded polypeptide is a single-chain variable fragment (scFv), a peptide, a fibronectin type III domain (FN3 domain), a single-domain antibody (sdAb, also known as a nanobody), an affibody, a darpin, a fynomer, an OBody, or an avimer.
16. A method of displaying a polypeptide on a substrate, comprising:
   i) providing a first nucleic acid comprising an antisense sequence encoding a single-chain variable fragment (scFv), wherein the first nucleic acid is immobilised on a substrate, and wherein the first nucleic acid is oriented such that the 5′ end is proximal and the 3′ end is distal to the point of immobilisation;
   ii) generating a second nucleic acid that is complementary to the first nucleic acid, wherein the generation of the second nucleic acid comprises:
      contacting the first nucleic acid with a nucleic acid polymerase under conditions suitable for RNA polymerisation, wherein
      the primer for polymerisation is immobilised on the substrate such a bridge is formed during polymerisation, and
      the product of the polymerisation is a chain of RNA nucleotides that is immobilised on the substrate via the primer:
   iii) removing the first nucleic acid to result in display of the second nucleic acid on the substrate; and
   iv) contacting the second nucleic acid with a ribosome under conditions suitable for translation of the encoded scFv, wherein the conditions of step iv) comprise trimethylamine N-oxide (TMAO).
17. The method of any one of clauses 14 to 16, wherein the TMAO is at a concentration of 0.05-1.5 M, 0.05-1.2M, or 4 M.
18. The method of any one of clauses 14 to 17, wherein the ribosome-polypeptide complex is stabilised by the application of a ribosome display buffer, and wherein the ribosome display buffer comprises a magnesium concentration which is:
   greater than 7 mM MgCl₂; or
   equivalent to 8, 9, 10, 15, 20, 25, 30, 40, 50, 60, 70, 80, 90, or 100 mM MgCl₂ or MgAc: or
   equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgCl₂: or
   equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgAc.
19. The method of any one of clauses 1 to 18, wherein the second nucleic acid is an RNA molecule and wherein a plurality of first nucleic acids encoding a plurality of polypeptides are provided in step i), such that a display library is created by the method.
20. The method of any one of clauses 1 to 19, wherein the first nucleic acid immobilised on the substrate as provided in step i) is generated by:
   1) providing a template nucleic acid;
   2) hybridising the template nucleic acid to a primer immobilised to a substrate:
   3) contacting the hybridised template nucleic acid with a polymerase under conditions suitable for the extension of the immobilised primer to synthesise the first nucleic acid which is a chain of nucleotides that are complementary to the template;
   4) performing bridge amplification of the first nucleic acid to generate clusters of the first nucleic acid; and
   5) sequencing at least a part of the first nucleic acid: optionally wherein the bridge amplification:
   comprises 32-35 amplification cycles,
   has an extension time of 60-120 seconds per cycle,
   comprises the use of an amplification buffer comprising Mg at a concentration equivalent to 2-6 mM of MgSO₄, and/or
comprises the use of a denaturation buffer comprising 95-99.9% Formamide, optionally 1-10 mM NaOH, and optionally 1-5 mM EDTA.
21. A method of preparing clusters of substrate-bound nucleic acids, comprising:
   1) providing a template nucleic acid;
   2) hybridising the template nucleic acid to a primer immobilised to a substrate;
   3) contacting the hybridised template nucleic acid with a polymerase under conditions suitable for the extension of the immobilised primer to synthesise the first nucleic acid which is a chain of nucleotides that are complementary to the template; and
   4) performing bridge amplification of the first nucleic acid to generate clusters of the first nucleic acid, wherein the bridge amplification is carried out for 32-35 amplification cycles, has an extension time of 60-120 seconds per cycle, comprises the use of an amplification buffer comprising Mg at a concentration equivalent to 2-6 mM of MgSO₄, and comprises the use of a denaturation buffer comprising 95-99.9% Formamide, optionally 1-10 mM NaOH, and optionally 1-5 mM EDTA.
22. A substrate displaying:
   (i) an RNA molecule which is obtained or obtainable by the methods of any one of clauses 1 to 3, 6 to 13, or 19 to 20;
   (ii) an XNA molecule which is obtained or obtainable by the methods of any one of clauses 1, 4 to 13, or 20; or
   (iii) a polypeptide molecule which is obtained or obtainable by the methods of any one of clauses 14 to 20.
23. Use of a nucleic acid polymerase to extend a DNA primer immobilised on a substrate to synthesise a non-DNA nucleic acid molecule that is complementary to a single-stranded nucleic acid template.
24. The use of clause 23, wherein the nucleic acid polymerase comprises an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide.
25. The use of clause 23 or clause 24, wherein the nucleic acid polymerase comprises a sequence that has at least 80%, 90%, 95%, 99%, or 100% identity to the amino acid sequence of SEQ ID NO: 3, and residues 93, 141, 143, 409, 485, and 664 are invariant.

1. A method of displaying a non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
a) contacting the first nucleic acid with a nucleic acid polymerase under conditions suitable for polymerisation, wherein
the primer for polymerisation is a DNA primer immobilised on the substrate such a bridge is formed during polymerisation,
the product of the polymerisation is a chain of non-DNA nucleotides that is immobilised on the substrate via the primer, and
the nucleic acid polymerase is a polymerase capable of acting upon a DNA primer to synthesise a non-DNA nucleic acid molecule that is complementary to a single-stranded nucleic acid template; and
iii) removing the first nucleic acid to result in display of the second nucleic acid on the substrate.

2. The method of claim 1, wherein the second nucleic acid is an RNA molecule.

3. The method of claim 2, wherein the nucleic acid polymerase comprises an amino acid sequence having at least 36% identity to the amino acid sequence of SEQ ID NO: 1, wherein said amino acid sequence comprises a Y409 and an E664 mutation relative to the amino acid sequence of SEQ ID NO: 1.

4. The method of claim 3, wherein the Y409 mutation is Y409N or Y409G and the E664 mutation is E664K or E664Q.

5. The method of claim 3, wherein the Y409 mutation is Y409G and the E664 mutation is E664K.

6. The method of claim 3, wherein the amino acid sequence of the nucleic acid polymerase comprises SEQ ID NO: 3.

7. The method of claim 1, wherein the second nucleic acid is an XNA molecule.

8. The method of claim 7, wherein the XNA molecule comprises an arabinonucleotide, an arabinonucleic acid (ANA) nucleotide, a 2′-Fluoro-arabinonucleic acid (FANA) nucleotide, a 2′-O-methyl ribonucleic acid (2′OMe) nucleotide, a 2′-O-methoxyethyl (MOE) nucleic acid nucleotide, a phosphorothioate 2′-O-methoxyethyl (PS-MOE) nucleotide, a phosphorodiamidate morpholino nucleotide, a locked nucleic acid (LNA) nucleotide, a P-alkyl phosphonate nucleic acid (phNA) nucleotide, a threose nucleic acid (TNA) nucleotide, a hexitol nucleic acid (HNA) nucleotide, a 2′ hydroxy-hexitol (AtNA) nucleotide, a cyclohexene nucleic acid (CeNA) nucleotide, or a 3′ deoxi-DNA (2′-5′) nucleotide.

9. The method of any preceding claim, wherein the nucleic acid polymerase comprises an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide.

10. The method of claim 9, wherein the amino acid sequence of the nucleic acid polymerase comprises one or more, or all, of the following mutations: V93Q, D141A, E143A, and A485L.

11. The method of any one of claims 1 to 10, wherein the polymerase is TGK, TGLLK, 2M, Bst, RT521, 6G12, 6G12521, C7, PGLVV, PGLVVWA, D4K, or a variant thereof.

12. The method of any one of claims 1 to 11, wherein after step ii) a), the method comprises cleaving the first nucleic acid and linearizing the bridge.

13. The method of claim 12, further comprising re-contacting the linearized product with the nucleic acid polymerase under conditions suitable for polymerisation.

14. A method of displaying a non-DNA nucleic acid molecule on a substrate, comprising:
i) providing a first nucleic acid immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
a) contacting the first nucleic acid with a nucleic acid polymerase under conditions suitable for polymerisation, wherein
the primer for polymerisation is immobilised on the substrate such a bridge is formed during polymerisation, and
the product of the polymerisation is a chain of non-DNA nucleotides that is immobilised on the substrate via the primer;
b) cleaving the first nucleic acid and linearizing the bridge; and
c) contacting the linearized product of step b) with a nucleic acid polymerase under conditions suitable for polymerisation; and
iii) removing the first nucleic acid to result in display of the second nucleic acid on the substrate.

15. The method of claim 14, wherein the second nucleic acid is an RNA molecule.

16. The method of any one of claims 12 to 15, wherein the bridge is denatured by temperature.

17. The method of any one of claims 12 to 16, wherein the first nucleic acid is cleaved with formamidopyrimidine DNA glycosylase (Fpg) at an 8-oxoguanine site.

18. The method of claim 17, wherein a third nucleic acid is annealed to the first nucleic acid at the 8-oxoguanine site before cleavage with Fpg.

19. The method of any one of claims 1 to 18, wherein step ii) a) comprises at least 5, 10, 12, 15, 20, or 25 cycles of bridged polymerisation.

20. The method of any one of claims 1 to 19, wherein the first nucleic acid is removed in step iii) by contacting the first nucleic acid with a denaturation reagent.

21. The method of claim 20, wherein the denaturation reagent is a buffer comprising:
1-500 mM NaOH and 0-20 mM EDTA; or
100 mM NaOH and 5 mM EDTA.

22. The method of any one of claims 1 to 21, wherein the second nucleic acid is an RNA molecule and encodes a polypeptide, and further comprising:
iv) contacting the second nucleic acid with a ribosome under conditions suitable for translation of the encoded polypeptide.

23. The method of claim 22, wherein the conditions of step iv) comprise trimethylamine N-oxide (TMAO).

24. The method of claim 22 or claim 23, wherein the encoded polypeptide is an antibody fragment or an enzyme.

25. The method of any one of claims 22 to 24, wherein encoded polypeptide is a single-chain variable fragment (scFv), a peptide, a fibronectin type III domain (FN3 domain), a single-domain antibody (sdAb, also known as a nanobody), an affibody, a darpin, a fynomer, an OBody, or an avimer.

26. A method of displaying a polypeptide on a substrate, comprising:
i) providing a first nucleic acid comprising an antisense sequence encoding a single-chain variable fragment (scFv), wherein the first nucleic acid is immobilised on a substrate, and wherein the first nucleic acid is oriented such that the 5′ end is proximal and the 3′ end is distal to the point of immobilisation;
ii) generating a second nucleic acid that is complementary to the first nucleic acid, wherein the generation of the second nucleic acid comprises:
contacting the first nucleic acid with a nucleic acid polymerase under conditions suitable for RNA polymerisation, wherein
the primer for polymerisation is immobilised on the substrate such a bridge is formed during polymerisation, and
the product of the polymerisation is a chain of RNA nucleotides that is immobilised on the substrate via the primer:
iii) removing the first nucleic acid to result in display of the second nucleic acid on the substrate; and
iv) contacting the second nucleic acid with a ribosome under conditions suitable for translation of the encoded scFv, wherein the conditions of step iv) comprise trimethylamine N-oxide (TMAO).

27. The method of any one of claims 23 to 26, wherein the TMAO is at a concentration of 0.05-1.5 M, 0.05-1.2M, or 4 M.

28. The method of any one of claims 22 to 27, wherein the ribosome-polypeptide complex is stabilised by the application of a ribosome display buffer

29. The method of claim 28, and wherein the ribosome display buffer comprises a magnesium concentration which is:
greater than 7 mM MgCl₂; or
equivalent to 8, 9, 10, 15, 20, 25, 30, 40, 50, 60, 70, 80, 90, or 100 mM MgCl₂ or MgAc: or
equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgCl₂: or
equivalent to from 8 to 100 mM, from 10 to 90 mM, from 15 to 85 mM, from 20 to 80 mM, from 25 to 75 mM, from 30 to 70 mM, from 35 to 65 mM, from 40 to 60 mM, or from 45 to 55 mM MgAc.

30. The method of any one of claims 1 to 29, wherein the second nucleic acid is an RNA molecule and wherein a plurality of first nucleic acids encoding a plurality of polypeptides are provided in step i), such that a display library is created by the method.

31. The method of any one of claims 1 to 30, wherein the first nucleic acid immobilised on the substrate as provided in step i) is generated by:
1) providing a template nucleic acid;
2) hybridising the template nucleic acid to a primer immobilised to a substrate;
3) contacting the hybridised template nucleic acid with a polymerase under conditions suitable for the extension of the immobilised primer to synthesise the first nucleic acid which is a chain of nucleotides that are complementary to the template;
4) performing bridge amplification of the first nucleic acid to generate clusters of the first nucleic acid; and
5) sequencing at least a part of the first nucleic acid.

32. The method of claim 31, wherein the bridge amplification:
comprises 32-35 amplification cycles,
has an extension time of 60-120 seconds per cycle,
comprises the use of an amplification buffer comprising Mg at a concentration equivalent to 2-6 mM of MgSO₄, and/or
comprises the use of a denaturation buffer comprising 95-99.9% Formamide, optionally 1-10 mM NaOH, and optionally 1-5 mM EDTA.

33. The method of claim 32, wherein the bridge amplification:
comprises 32 amplification cycles,
has an extension time of 60 seconds per cycle,
comprises the use of an amplification buffer comprising Mg at a concentration equivalent to 6 mM of MgSO₄, and/or
comprises the use of a denaturation buffer comprising 98% Formamide, 10 mM NaOH, and 1 mM EDTA.

34. A method of preparing clusters of substrate-bound nucleic acids, comprising:
1) providing a template nucleic acid;
2) hybridising the template nucleic acid to a primer immobilised to a substrate;
3) contacting the hybridised template nucleic acid with a polymerase under conditions suitable for the extension of the immobilised primer to synthesise the first nucleic acid which is a chain of nucleotides that are complementary to the template; and
4) performing bridge amplification of the first nucleic acid to generate clusters of the first nucleic acid, wherein the bridge amplification is carried out for 32-35 amplification cycles, has an extension time of 60-120 seconds per cycle, comprises the use of an amplification buffer comprising Mg at a concentration equivalent to 2-6 mM of MgSO₄, and comprises the use of a denaturation buffer comprising 95-99.9% Formamide, optionally 1-10 mM NaOH, and optionally 1-5 mM EDTA.

35. The method of claim 34, wherein the bridge amplification:
comprises 32 amplification cycles,
has an extension time of 60 seconds per cycle,
comprises the use of an amplification buffer comprising Mg at a concentration equivalent to 6 mM of MgSO₄, and/or
comprises the use of a denaturation buffer comprising 98% Formamide, 10 mM NaOH, and 1 mM EDTA.

36. A substrate displaying a non-DNA nucleic acid molecule which is obtained or obtainable by the methods of any one of claims 1 to 21 or 30 to 33.

37. A substrate displaying an RNA molecule which is obtained or obtainable by the methods of any one of claims 1 to 6, 9 to 21, or 30 to 33.

38. A substrate displaying an XNA molecule which is obtained or obtainable by the methods of any one of claims 1, 7 to 14, 16 to 21, or 31 to 33.

39. A substrate displaying a polypeptide molecule which is obtained or obtainable by the methods of any one of claims 22 to 33.

40. Use of a nucleic acid polymerase to extend a DNA primer immobilised on a substrate to synthesise a non-DNA nucleic acid molecule that is complementary to a single-stranded nucleic acid template.

41. The use of claim 40, wherein the nucleic acid polymerase comprises an amino acid sequence having at least 36% similarity or identity to the amino acid sequence of SEQ ID NO: 1 and comprises a Y409 and an E664 mutation, and wherein an RNA molecule is polymerised that is complementary to the nucleic acid template.

42. The use of claim 41, wherein the nucleic acid polymerase comprises a sequence that has at least 80%, 90%, 95%, 99%, or 100% identity to the amino acid sequence of SEQ ID NO: 3, and residues 93, 141, 143, 409, 485, and 664 are invariant.

43. The use according to any one of claims 40 to 42, wherein the nucleic acid polymerase comprises an amino acid sequence having at least 36%, 50%, 60%, 70%, 80%, 90%, 95%, 99%, or 100% similarity or identity to the amino acid sequence of SEQ ID NO: 1, and further comprises mutations allowing the polymerisation of at least one type of XNA nucleotide or RNA nucleotide.

44. The use according to any one of claims 40 to 43, wherein the amino acid sequence of the nucleic acid polymerase comprises one or more, or all, of the following mutations: V93Q, D141A, E143A, and A485L.

45. The use according to any one of claims 40 to 44, wherein the polymerase is TGK, TGLLK, 2M, Bst, RT521, 6G12, 6G12521, C7, PGLVV, PGLVVWA, D4K, or a variant thereof.

46. A method of screening a substrate displaying a plurality of biomolecules, wherein the substrate is according to any one of claims 36 to 39, and wherein the biomolecules form a library.

47. A method according to any one of claims 1 to 33, where the method further comprises screening the displayed non-DNA nucleic acid molecule or polypeptide molecule.

48. The method of claim 46 or 47, wherein the screening comprises measuring the affinity for a ligand or a target molecule, or measuring an enzymatic function, of the displayed biomolecules, non-DNA nucleic acid molecule, or polypeptide molecule.


SEQUENCE LISTING (192 sequences)

SEQ ID NO: 1 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 2 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 3 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 4 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYPDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPTQHLVIHKQITRALNDYKAIGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 5 - DNA, 58 bp, synthetic construct
  source 1..58: mol_type = other DNA
aatgatacggcgaccaccgagatctacactctttccctacacgacgctcttccgatct

SEQ ID NO: 6 - DNA, 27 bp, synthetic construct
  source 1..27: mol_type = other DNA
cattacaaacgacaccctaaacaaatc

SEQ ID NO: 7 - DNA, 27 bp, synthetic construct
  source 1..27: mol_type = other DNA
tattttaataattaaggaggtatatac

SEQ ID NO: 8 - DNA, 288 bp, synthetic construct
  source 1..288: mol_type = other DNA
tatatggctagtggtgccgaatttgggtcaggtggccagaagcaagctgaggaagccgctgccaaggctgccgcagatgcaaaggcaaaagccgaggcagacgctaaagctgcggaagaggctgcgaaaaaggcagcagccgatgctaagaaaaaagcggaggcggaagccgccaaagccgcagcggaagctcagaagaaggccgaggcagctgccgcggcacttaaaaagaaagctgaagcagcagaagcggctgcggccgaggctcgtaagaaggcggcaacagag

SEQ ID NO: 9 - DNA, 165 bp, synthetic construct
  source 1..165: mol_type = other DNA
tatatggctagtggtgccgaatttgggtcaggtggccagaagcaagctgaggaagccgctgccaaggctgccgcagatgcaaaggcaaaagccgaggcagacgctaaagctgcggaagaggctgcgaaaaaggcagcagccgatgctaagaaaaaagcggaggcg

SEQ ID NO: 10 - DNA, 27 bp, synthetic construct
  source 1..27: mol_type = other DNA
tcctgttagactcctcaatgcaagctg

SEQ ID NO: 11 - DNA, 57 bp, synthetic construct
  source 1..57: mol_type = other DNA
gatcggaagagcacacgtctgaactccagtcacatctcgtatgccgtcttctgcttg

SEQ ID NO: 12 - DNA, 768 bp, synthetic construct
  source 1..768: mol_type = other DNA
atgcaggtacagcttgtgcagtctggggctgaagtcaagaaaccgggtgaaagtctgaaaattagttgtaagggctcaggatattcgtttacaagctattggattgcgtgggttcgccagatgcctggtaagggattagagtatatgggccttatctacccgggcgactccgataccaaatactccccaagttttcagggtcaagtaaccattagcgtggacaagtctgtgtccaccgcatacctgcagtggtcgagcttgaagccctctgattctgcggtatacttctgtgctcgtcatgacgtcgcctattgttccagtagcaattgcgcaaagtggcctgagtatttccaacattggggacaagggacccttgtcacagttagtagcgggggtggtggatccggcgggggcggctcaggtggaggtggaagccagtcagtattaactcaaccgcctagtgtctcagcagccccgggccagaaagttacgattagctgttctggatcaagttcaaacatcggaaacaactatgtgtcttggtatcaacagctgccagggacagctcctaaattgctgatctatggccatacaaaccgcccagcaggtgtacccgaccgtttctccggatcgaaatcgggtacatccgcctcgttagctatttctgggtttcgcagcgaagatgaagccgattattattgtgcgagttgggactatactctttcagggtgggtgtttggtggagggacgaagcttactgtgcttggg

SEQ ID NO: 13 - DNA, 768 bp, synthetic construct
  source 1..768: mol_type = other DNA
atgcaggtgcagttggtccaatcaggcgccgaggtaaagaagccgggggaatctcttaaaatttcctgtaagggcagtggctactcctttactagttattggattgcctgggttcgccaaatgcctgggaagggtcttgagtatatgggactgatctatcccggtgattctgacaccaaatactcgccaagtttccagggacaagttacaatttctgttgataagtcggtatctaccgcctacttgcaatggtcatccttaaaaccatccgactcggcggtgtacttttgtgcccgccatgatgtggggtactgctcgtcaagcaactgcgcgaaatggcccgagtattttcagcattgggggcagggcactttagtaactgtttcatcaggtggaggagggagcggtggaggcgggagcggtggtggcggttctcagtctgttttaactcaacctccgtcagtttctgctgccccaggtcaaaaagtcactatcagttgttcaggatcgagttcgaacattggtaataactacgtatcctggtatcaacaattacccggaaccgcccctaagctgttaatctatggtcacacgaatcgccccgccggagttccggaccgcttcagcgggagtaaatctggaacgtcagcctctctggcaatctcaggctttcgttcagaggacgaggctgactactattgtgcggcttgggacgactctctttccggctgggtctttggtggaggtacaaaactgacggtgttaggt

SEQ ID NO: 14 - DNA, 768 bp, synthetic construct
  source 1..768: mol_type = other DNA
atgcaggtccagcttgtccagtcaggtgcagaggtcaagaagcctggggagtcgctgaagatctcatgcaaggggtcaggatacagtttcacgagttattggattgcatgggtgcgtcaaatgccaggcaaagggctggagtatatggggttgatctatccgggcgatagcgatacgaaatactcacccagttttcagggtcaagtcactatttcggttgataaatccgttagtactgcatacttacaatggagctcattaaaaccctctgactcggctgtctacttttgtgcccgccacgacgtgggatattgctcgagttctaattgcgcgaagtggccagagtattttcagcactgggggcaggggacactggtgacggtaagttcaggcgggggtgggtctggtggaggaggctctgggggaggaggatctcagtctgttttgacgcaacctcccagtgtgtctgccgcaccgggtcaaaaggtgaccatttcgtgctccgggtcatcttctaacatcgggaacaattatgtgtcctggtatcagcagcttccagggactgcacccaaactgttaatctacgatcatacgaatcgtcccgctggtgtgccagaccgcttctctgggagcaagtcaggcacatctgcatccttagccattagtggcttccgcagtgaagatgaagcagactattactgtgcgagttgggattatacattaagcgggtgggtatttggaggcggcacaaagttaacagtgttaggt

SEQ ID NO: 15 - DNA, 768 bp, synthetic construct
  source 1..768: mol_type = other DNA
atgcaggtgcaacttgtccagagcggtgctgaggtaaaaaaacctggcgaatctttgaaaatcagctgtaagggctccggttactcatttacttcatactggattgcgtgggtccgccaaatgcctggaaagggattagaatatatggggttaatctatccaggcgattcagacacgaaatattctccaagctttcagggtcaagtcaccatctctgtcgacaaaagcgtttcgacagcttacctgcaatggtctagcctgaagccctcggacagtgccgtatacttttgtgcccgtcatgacgttggatactgtactgaccgcacctgtgctaaatggcctgaatatttccagcattggggtcaggggacgttagttacggtctcatcaggtggcggaggatccggtggtggtggatccggtggaggggggtcacaatccgtattgacgcagcctccctcagtaagtgcagcgcctggacagaaagtgactatctcttgttccggatcctctagtaatattgggaataattacgtgagttggtaccagcagctgcccggcaccgcacctaagttacttatttacgatcataccaaccgtccggcgggcgtaccagaccgtttcagtggcagcaagagcggaacctctgcgagcttagcaatcagtggctttcgctcagaggacgaggcagattattactgtgcatcatgggattatactctgtcaggatgggttttcggcggcgggacaaaattaacagtcttgggt

SEQ ID NO: 16 - DNA, 768 bp, synthetic construct
  source 1..768: mol_type = other DNA
atgcaggtacaattggtccagtcaggcgcggaggtcaaaaaaccaggggagtctttaaagatttcgtgcaaaggttccggatattctttcacttcgtactggatcgcgtgggtccgccaaatgccaggcaaggggttagaatatatgggactgatttatccgggggattcggacaccaagtactccccatcgttccaaggacaagtgactatcagcgtcgacaaatctgtgtcgaccgcgtacttgcagtggtccagccttaagccaagtgactcggccgtttacttctgtgctcgccatgacgttgggtactgtaccgaccgtacatgcgccaaatggccggagtggcttggtgtttgggggcaagggactcttgttactgtttctagtggaggcggggggtcaggtgggggtggctctggtggcgggggatcgcaatcagttttgactcaaccaccttctgtttctgccgcgcccggacagaaggtgacgattagctgcagcggatcatcgtcaaacatcggcaataactatgtttcctggtatcaacagttgccgggaaccgcccccaaattgttgatctatgaccatactaaccgccccgccggagtgcctgatcgcttctcagggtctaagtccggcacgtccgcaagcttggccatttccggattccgcagtgaggacgaagccgactattattgcgcgtcttgggactacacgctttctggatgggtgtttgggggaggcactaaactgaccgtccttggg

SEQ ID NO: 17 - DNA, 732 bp, synthetic construct
  source 1..732: mol_type = other DNA
atggaggttcagctggttgaaagcggtggtggtctggttcagcctggtggtagcctgcgtctgagctgtgcagcaagcggttttaatattaaagatacctatattcattgggtgcgtcaggcaccgggtaaaggtctggaatgggttgcacgtatttatccgaccaatggttatacccgttatgcagatagcgtgaaaggtcgttttaccattagcgcagataccagcaaaaataccgcatatctgcagatgaatagcctgcgtgcagaagataccgcagtttattattgtagccgttggggtggtgatggtttttatgcaatggatgtttggggtcagggcaccctggttaccgttagcagtggtggtggtggtagcggtggtggcggttctggtggcggtggtagtaccgatattcagatgacccagagcccgagcagcctgagcgcaagcgttggtgatcgtgttaccattacctgtcgtgcaagccaggatgttaataccgcagttgcatggtatcagcagaaaccgggtaaagcaccgaaactgctgatttatagcgcatcttttctggaaagcggtgttccgagccgttttagcggtagccgtagcggcaccgattttaccctgaccattagcagcctgcagccggaagattttgcaacctattattgtcagcagcattataccacacctccgacctttggccagggcaccaaagttgaaattaaa

SEQ ID NO: 18 - DNA, 33 bp, synthetic construct
  source 1..33: mol_type = other DNA
gtgactggagttcagacgtgtgctcttccgatc

SEQ ID NO: 19 - DNA, 37 bp, synthetic construct
  source 1..37: mol_type = other DNA
gaactccagtcacatctcgtatgccgtcttctgcttg

SEQ ID NO: 20 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADGDGFLATIPGADAETVKKKAKEFLDYINAKLPGLLELEYEGFYKRGFFVTKAKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 21 - DNA, 20 bp, synthetic construct
  source 1..20: mol_type = other DNA
gcctgagctgcgcggcgagc

SEQ ID NO: 22 - DNA, 23 bp, synthetic construct
  source 1..23: mol_type = other DNA
gccaggcgccgggcaaagaacgc

SEQ ID NO: 23 - DNA, 30 bp, synthetic construct
  source 1..30: mol_type = other DNA
ccggaagataccgcggtgtattattgcgcg

SEQ ID NO: 24 - DNA, 28 bp, synthetic construct
  source 1..28: mol_type = other DNA
gtccccaggacagacagccagcatcacc

SEQ ID NO: 25 - DNA, 35 bp, synthetic construct
  source 1..35: mol_type = other DNA
ccgggacccaggctatggatgaggctgagtattac

SEQ ID NO: 26 - DNA, 34 bp, synthetic construct
  source 1..34: mol_type = other DNA
gccctctgattctgcggtatacttctgtgctcgt

SEQ ID NO: 27 - DNA, 40 bp, synthetic construct
  source 1..40: mol_type = other DNA
attaaggaggtatatacatgcaggtgcagctgcaggaaag

SEQ ID NO: 28 - DNA, 55 bp, synthetic construct
  source 1..55: mol_type = other DNA
tgacccaaattcggcaccactagccatataagcgtaatctggaacatcgtatggg

SEQ ID NO: 29 - DNA, 20 bp, synthetic construct
  source 1..20: mol_type = other DNA
aatgatacggcgaccaccga

SEQ ID NO: 30 - DNA, 24 bp, synthetic construct
  source 1..24: mol_type = other DNA
caagcagaagacggcatacgagat

SEQ ID NO: 31 - DNA, 38 bp, synthetic construct
  source 1..38: mol_type = other DNA
attaaggaggtatatacatgcaggtacagcttgtgcag

SEQ ID NO: 32 - DNA, 22 bp, synthetic construct
  source 1..22: mol_type = other DNA
acgagcacagaagtataccgca

SEQ ID NO: 33 - DNA, 23 bp, synthetic construct
  source 1..23: mol_type = other DNA
tggggacaagggacccttgtcac

SEQ ID NO: 34 - DNA, 53 bp, synthetic construct
  source 1..53: mol_type = other DNA
tgacccaaattcggcaccactagccatatacccaagcacagtaagcttcgtcc

SEQ ID NO: 35 - DNA, 100 bp, synthetic construct
  source 1..100: mol_type = other DNA
cggtatacttctgtgctcgtnnsnnsnnsnnstattgttccagtagcaattgcgcaaagtggcctgagtatttccaacattggggacaagggacccttgt

SEQ ID NO: 36 - DNA, 100 bp, synthetic construct
  source 1..100: mol_type = other DNA
cggtatacttctgtgctcgtcatgacgtcnnsnnsnnsnnsagtagcaattgcgcaaagtggcctgagtatttccaacattggggacaagggacccttgt

SEQ ID NO: 37 - DNA, 100 bp, synthetic construct
  source 1..100: mol_type = other DNA
cggtatacttctgtgctcgtcatgacgtcgcctattgtnnsnnsnnsnnstgcgcaaagtggcctgagtatttccaacattggggacaagggacccttgt

SEQ ID NO: 38 - DNA, 100 bp, synthetic construct
  source 1..100: mol_type = other DNA
cggtatacttctgtgctcgtcatgacgtcgcctattgttccagtagcnnsnnsnnsnnstggcctgagtatttccaacattggggacaagggacccttgt

SEQ ID NO: 39 - DNA, 100 bp, synthetic construct
  source 1..100: mol_type = other DNA
cggtatacttctgtgctcgtcatgacgtcgcctattgttccagtagcaattgcgcannsnnsnnsnnstatttccaacattggggacaagggacccttgt

SEQ ID NO: 40 - DNA, 100 bp, synthetic construct
  source 1..100: mol_type = other DNA
cggtatacttctgtgctcgtcatgacgtcgcctattgttccagtagcaattgcgcaaagtggcctnnsnnsnnsnnscattggggacaagggacccttgt

SEQ ID NO: 41 - protein, 9 aa, synthetic construct
GTIFDTLSM

SEQ ID NO: 42 - protein, 14 aa, synthetic construct
EFVATIADGSTTYY

SEQ ID NO: 43 - protein, 16 aa, synthetic construct
AAVQYPSYTWYWTYNY

SEQ ID NO: 44 - protein, 9 aa, synthetic construct
GNIFRSRYM

SEQ ID NO: 45 - protein, 14 aa, synthetic construct
ELVAGIAGGSITNY

SEQ ID NO: 46 - protein, 16 aa, synthetic construct
AAASDWVDPYYDDYYY

SEQ ID NO: 47 - protein, 9 aa, synthetic construct
GYIFYSIAM

SEQ ID NO: 48 - protein, 14 aa, synthetic construct
ELVASIATGTNTYY

SEQ ID NO: 49 - protein, 12 aa, synthetic construct
AVRWSYYIRLYY

SEQ ID NO: 50 - protein, 9 aa, synthetic construct
GSIFVSIEM

SEQ ID NO: 51 - protein, 14 aa, synthetic construct
EFVASIGSGTSTYY

SEQ ID NO: 52 - protein, 16 aa, synthetic construct
AVQRSFESYGYWGHVY

SEQ ID NO: 53 - protein, 9 aa, synthetic construct
GSIFYYTDM

SEQ ID NO: 54 - protein, 14 aa, synthetic construct
EFVASIGYGGITYY

SEQ ID NO: 55 - protein, 16 aa, synthetic construct
AVQRSFESYGYWGHVY

SEQ ID NO: 56 - protein, 9 aa, synthetic construct
GSISRDPDM

SEQ ID NO: 57 - protein, 14 aa, synthetic construct
EFVASISGGTTTYY

SEQ ID NO: 58 - protein, 16 aa, synthetic construct
AVQRSFESYGYWGHVY

SEQ ID NO: 59 - protein, 9 aa, synthetic construct
GSIFRWKFM

SEQ ID NO: 60 - protein, 14 aa, synthetic construct
ELVATISGGASTYY

SEQ ID NO: 61 - protein, 12 aa, synthetic construct
AVSTPTVYWFWY

SEQ ID NO: 62 - protein, 9 aa, synthetic construct
GNIFQIKYM

SEQ ID NO: 63 - protein, 14 aa, synthetic construct
EFVAAINDGGTTNY

SEQ ID NO: 64 - protein, 20 aa, synthetic construct
AVGGRNDLVWVWWRPWVYIY

SEQ ID NO: 65 - protein, 9 aa, synthetic construct
GNISYTADM

SEQ ID NO: 66 - protein, 14 aa, synthetic construct
EFVASITWGGTTYY

SEQ ID NO: 67 - protein, 16 aa, synthetic construct
AVQRSFESYGYWGHVY

SEQ ID NO: 68 - protein, 9 aa, synthetic construct
GTIFYYFYM

SEQ ID NO: 69 - protein, 14 aa, synthetic construct
EFVASINYGASTYY

SEQ ID NO: 70 - protein, 12 aa, synthetic construct
AAESYTSVYYSY

SEQ ID NO: 71 - protein, 9 aa, synthetic construct
GTIFVWSDM

SEQ ID NO: 72 - protein, 14 aa, synthetic construct
EFVASIGYGGSTYY

SEQ ID NO: 73 - protein, 16 aa, synthetic construct
AVQRPFESYGYWGHVY

SEQ ID NO: 74 - protein, 9 aa, synthetic construct
GNISPTADM

SEQ ID NO: 75 - protein, 14 aa, synthetic construct
EFVASISYGASTYY

SEQ ID NO: 76 - protein, 16 aa, synthetic construct
AVQRSFESYGYWGHVY

SEQ ID NO: 77 - protein, 9 aa, synthetic construct
GSISRTADM

SEQ ID NO: 78 - protein, 14 aa, synthetic construct
EFVAAIAPGAITYY

SEQ ID NO: 79 - protein, 20 aa, synthetic construct
AVQRSFESYGYWGHVYWGQG

SEQ ID NO: 80 - protein, 9 aa, synthetic construct
GSIFHDFFM

SEQ ID NO: 81 - protein, 14 aa, synthetic construct
EFVAGITEGGSTYY

SEQ ID NO: 82 - protein, 12 aa, synthetic construct
AVALKYADYFGY

SEQ ID NO: 83 - protein, 9 aa, synthetic construct
GTISSQKLM

SEQ ID NO: 84 - protein, 14 aa, synthetic construct
EFVAAINDGATTNY

SEQ ID NO: 85 - protein, 16 aa, synthetic construct
AAYYDVRGYYWGTFGY

SEQ ID NO: 86 - protein, 9 aa, synthetic construct
GYIFYSIAM

SEQ ID NO: 87 - protein, 14 aa, synthetic construct
ELVASIATGTNTYY

SEQ ID NO: 88 - protein, 20 aa, synthetic construct
AVTYPIRVSAPYWYGAYHRY

SEQ ID NO: 89 - protein, 9 aa, synthetic construct
GSIFHDFFM

SEQ ID NO: 90 - protein, 14 aa, synthetic construct
EFVAGITEGGSTYY

SEQ ID NO: 91 - protein, 12 aa, synthetic construct
AVALQYADYFGY

SEQ ID NO: 92 - protein, 9 aa, synthetic construct
GTIFDTYDM

SEQ ID NO: 93 - protein, 14 aa, synthetic construct
ELVAAITPGANTYY

SEQ ID NO: 94 - protein, 12 aa, synthetic construct
AVTDPWDGRHSY

SEQ ID NO: 95 - protein, 9 aa, synthetic construct
GYIFQYGSM

SEQ ID NO: 96 - protein, 14 aa, synthetic construct
ELVAAIGGGGNTYY

SEQ ID NO: 97 - protein, 12 aa, synthetic construct
AVYNRVTRTLYY

SEQ ID NO: 98 - protein, 9 aa, synthetic construct
GTISYGADM

SEQ ID NO: 99 - protein, 14 aa, synthetic construct
EFVASIGSGSITYY

SEQ ID NO: 100 - protein, 16 aa, synthetic construct
AVQRSFESYGYWGHVY

SEQ ID NO: 101 - protein, 9 aa, synthetic construct
GNISYTADM

SEQ ID NO: 102 - protein, 14 aa, synthetic construct
EFVAGIGDGGITNY

SEQ ID NO: 103 - protein, 16 aa, synthetic construct
AVQRSFESYGYWGHVY

SEQ ID NO: 104 - protein, 9 aa, synthetic construct
GTISVFWDM

SEQ ID NO: 105 - protein, 14 aa, synthetic construct
EFVAGIGDGGTTNY

SEQ ID NO: 106 - protein, 12 aa, synthetic construct
AVDYVSGPSFIY

SEQ ID NO: 107 - protein, 9 aa, synthetic construct
GNIFGDFAM

SEQ ID NO: 108 - protein, 14 aa, synthetic construct
ELVASIGVGSSTYY

SEQ ID NO: 109 - protein, 12 aa, synthetic construct
AADDEYNWAHVY

SEQ ID NO: 110 - protein, 9 aa, synthetic construct
GSIFWAGYM

SEQ ID NO: 111 - protein, 14 aa, synthetic construct
EFVAAIDYGANTYY

SEQ ID NO: 112 - protein, 16 aa, synthetic construct
AVDRQPYYRRDNSHLY

SEQ ID NO: 113 - protein, 9 aa, synthetic construct
GNISADASM

SEQ ID NO: 114 - protein, 14 aa, synthetic construct
EFVAAIARGATTYY

SEQ ID NO: 115 - protein, 16 aa, synthetic construct
AVDQYDVTKRYIGHIY

SEQ ID NO: 116 - protein, 9 aa, synthetic construct
GNISTLDGM

SEQ ID NO: 117 - protein, 14 aa, synthetic construct
EFVATIAYGSSTYY

SEQ ID NO: 118 - protein, 20 aa, synthetic construct
AAADYSTIHIQYASYYGHQY

SEQ ID NO: 119 - protein, 9 aa, synthetic construct
GYIFNGDYM

SEQ ID NO: 120 - protein, 14 aa, synthetic construct
EFVASITIGGTTNY

SEQ ID NO: 121 - protein, 16 aa, synthetic construct
AADSLAGDPRQETHIY

SEQ ID NO: 122 - protein, 9 aa, synthetic construct
GNISRYWGM

SEQ ID NO: 123 - protein, 14 aa, synthetic construct
EFVASINYGTNTYY

SEQ ID NO: 124 - protein, 12 aa, synthetic construct
AVFVEDSPQHGY

SEQ ID NO: 125 - protein, 9 aa, synthetic construct
GYISVRPIM

SEQ ID NO: 126 - protein, 14 aa, synthetic construct
EFVAGITYGTTTYY

SEQ ID NO: 127 - protein, 12 aa, synthetic construct
AVESTYYVPYYY

SEQ ID NO: 128 - protein, 9 aa, synthetic construct
GYISVDSLM

SEQ ID NO: 129 - protein, 14 aa, synthetic construct
ELVATIGYGTNTNY

SEQ ID NO: 130 - protein, 20 aa, synthetic construct
AAGFYYRPWPGGVAHTDYDY

SEQ ID NO: 131 - protein, 9 aa, synthetic construct
GTISYYYDM

SEQ ID NO: 132 - protein, 14 aa, synthetic construct
ELVAAIDDGGTTYY

SEQ ID NO: 133 - protein, 12 aa, synthetic construct
AVTDPWDGRHSY

SEQ ID NO: 134 - protein, 9 aa, synthetic construct
GTIFASGSM

SEQ ID NO: 135 - protein, 14 aa, synthetic construct
EFVAAITWGGNTYY

SEQ ID NO: 136 - protein, 16 aa, synthetic construct
AVDWRYGGQRYHRHIY

SEQ ID NO: 137 - protein, 9 aa, synthetic construct
GTIFAGDEM

SEQ ID NO: 138 - protein, 14 aa, synthetic construct
EFVAGINNGSITNY

SEQ ID NO: 139 - protein, 16 aa, synthetic construct
AVYYQASDDGPKALLY

SEQ ID NO: 140 - protein, 9 aa, synthetic construct
GSISKYLFM

SEQ ID NO: 141 - protein, 14 aa, synthetic construct
EFVAAINRGGSTYY

SEQ ID NO: 142 - protein, 15 aa, synthetic construct
AVSGYRKGYNKRFSY

SEQ ID NO: 143 - protein, 9 aa, synthetic construct
GTIFGRYIM

SEQ ID NO: 144 - protein, 14 aa, synthetic construct
ELVAAIGNGATTYY

SEQ ID NO: 145 - protein, 12 aa, synthetic construct
AAYQSYRRKHYY

SEQ ID NO: 146 - protein, 9 aa, synthetic construct
GNISYTADM

SEQ ID NO: 147 - protein, 14 aa, synthetic construct
EFVASINTGGSTYY

SEQ ID NO: 148 - protein, 16 aa, synthetic construct
AVQRSFESYGYWGHVY

SEQ ID NO: 149 - protein, 14 aa, synthetic construct
EFVASIGRGTTYYA

SEQ ID NO: 150 - protein, 14 aa, synthetic construct
EFVASINYGASTYY

SEQ ID NO: 151 - protein, 15 aa, synthetic construct
CSGDKLGDKYASWYQ

SEQ ID NO: 152 - protein, 15 aa, synthetic construct
CQAWDSSSYVFGTGT

SEQ ID NO: 153 - protein, 15 aa, synthetic construct
CSGDNVGYYWASWYQ

SEQ ID NO: 154 - protein, 15 aa, synthetic construct
CLVSSSGSYVFGTGT

SEQ ID NO: 155 - protein, 15 aa, synthetic construct
CSGDNVDHYWASWYQ

SEQ ID NO: 156 - protein, 15 aa, synthetic construct
CLVSVDGSYVFGTGT

SEQ ID NO: 157 - protein, 15 aa, synthetic construct
CSGDEIERYWASWYQ

SEQ ID NO: 158 - protein, 15 aa, synthetic construct
CSGDNVQEYWASWYQ

SEQ ID NO: 159 - protein, 15 aa, synthetic construct
CSGDGIHRYWASWYQ

SEQ ID NO: 160 - protein, 15 aa, synthetic construct
CSGDDLGQYWASWYQ

SEQ ID NO: 161 - protein, 15 aa, synthetic construct
CLASENGSYVFGTGT

SEQ ID NO: 162 - protein, 15 aa, synthetic construct
CSGDGIERYWASWYQ

SEQ ID NO: 163 - protein, 15 aa, synthetic construct
CSGDKIGDVWASWYQ

SEQ ID NO: 164 - protein, 15 aa, synthetic construct
CSGDDLGSKWASWYQ

SEQ ID NO: 165 - protein, 15 aa, synthetic construct
CSGDAVGEKWASWYQ

SEQ ID NO: 166 - protein, 15 aa, synthetic construct
CLVSHEGSYVFGTGT

SEQ ID NO: 167 - protein, 15 aa, synthetic construct
CSGDHLGDLWASWYQ

SEQ ID NO: 168 - protein, 15 aa, synthetic construct
CSGDHLDKYWASWYQ

SEQ ID NO: 169 - protein, 15 aa, synthetic construct
CSGDRLGEKWASWYQ

SEQ ID NO: 170 - protein, 15 aa, synthetic construct
CSGDNIQNYWASWYQ

SEQ ID NO: 171 - protein, 15 aa, synthetic construct
CSGDRVGEKWASWYQ

SEQ ID NO: 172 - protein, 15 aa, synthetic construct
CSGDSIGEKWASWYQ

SEQ ID NO: 173 - protein, 15 aa, synthetic construct
CSGDKLGEKWASWYQ

SEQ ID NO: 174 - protein, 15 aa, synthetic construct
CSGDNLGDLWASWYQ

SEQ ID NO: 175 - protein, 15 aa, synthetic construct
CLASAGGSYVFGTGT

SEQ ID NO: 176 - protein, 15 aa, synthetic construct
CSGDDLGNYWASWYQ

SEQ ID NO: 177 - protein, 20 aa, synthetic construct
HDVAYCSSSNCAKWPEYFQH

SEQ ID NO: 178 - protein, 13 aa, synthetic construct
TAPKLLIYGHTNR

SEQ ID NO: 179 - protein, 7 aa, synthetic construct
SWDYTLS

SEQ ID NO: 180 - protein, 20 aa, synthetic construct
HDVGYCSSSNCAKWPEYFQH

SEQ ID NO: 181 - protein, 7 aa, synthetic construct
AWDDSLS

SEQ ID NO: 182 - protein, 13 aa, synthetic construct
TAPKLLIYDHTNR

SEQ ID NO: 183 - protein, 20 aa, synthetic construct
HDVGYCTDRTCAKWPEYFQH

SEQ ID NO: 184 - protein, 20 aa, synthetic construct
HDVGYCTDRTCAKWPEWLGV

SEQ ID NO: 185 - protein, 20 aa, synthetic construct
HDVAYCSSSRCLTWPEYFQH

SEQ ID NO: 186 - protein, 20 aa, synthetic construct
HDVGQCDSSNCAKWPEYFQH

SEQ ID NO: 187 - protein, 20 aa, synthetic construct
HDVAYCDTPTCAKWPEYFQH

SEQ ID NO: 188 - protein, 20 aa, synthetic construct
HDVYQCDSSNCAKTPEYFQH

SEQ ID NO: 189 - protein, 20 aa, synthetic construct
HDVGQCDSSNCAKWPEYGQW

SEQ ID NO: 190 - protein, 20 aa, synthetic construct
HSVGQCDSSNCAKWREYFQH

SEQ ID NO: 191 - protein, 20 aa, synthetic construct
HMVGQCDSSNMAKWPEAFSI

SEQ ID NO: 192 - protein, 20 aa, synthetic construct
HDVGACDSSNCTKYPEYGQT